(19) United States
(12) Patent Application Publication
(10) Pub. No.: US 2024/0199680 A1
(43) Pub. Date: Jun. 20, 2024

(54) SYNTHESIS OF FLUORINATED CYCLIC DINUCLEOTIDES

(71) Applicant: Merck Sharp & Dohme LLC, Rahway, NJ (US)

(72) Inventors: Chihui An, Scotch Plains, NJ (US); Patrick S. Fier, Monroe Township, NJ (US); Kaori Hiraga, North Bergen, NJ (US); Zhijian Liu, Kendall Park, NJ (US); Nicholas M. Marshall, Middleton, WI (US); John McIntosh, Metuchen, NJ (US); Steven P. Miller, Hampton, NJ (US); Jeffrey C. Moore, Westfield, NJ (US); Grant S. Murphy, Princeton, NJ (US); Jennifer V. Obligacion, Metuchen, NJ (US); Weilan Pan, Downington, PA (US); Feng Peng, Dayton, NJ (US); Nastaran Salehi Marzijarani, Edgewater, NJ (US); Matthew S. Winston, Maplewood, NJ (US)

(73) Assignee: Merck Sharp & Dohme LLC, Rahway, NJ (US)

(21) Appl. No.: 18/552,679

(22) PCT Filed: Mar. 28, 2022
(86) PCT No.: PCT/US2022/022092

§ 371 Date: Sep. 27, 2023

Related U.S. Application Data
(60) Provisional application No. 63/170,003, filed on Apr. 2, 2021.

(51) Int. Cl. C07H 21/00; C12N 9/12; C12P 19/36

(52) U.S. Cl. CPC C07H 21/00; C12N 9/1241; C12P 19/36

(57) ABSTRACT

The present invention relates to efficient processes useful in the preparation of fluorinated cyclic dinucleosides, such as [P(R)]-2′-deoxy-2′-fluoro-5′-O—[(R)-hydroxymercaptophosphinyl]-P-thio-β-D-arabino-adenylyl-(3′→5′)-3′-deoxy-3′-fluoroguanosine cyclic nucleotide, which is also known as (2R,5R,7R,8S,10R,12aR,14R,15S,15aR,16R)-7-(2-amino-6-oxo-1,6-dihydro-9H-purin-9-yl)-14-(6-amino-9H-purin-9-yl)-15,16-difluoro-2, 10-bis(sulfanyl)octahydro-2H,10H, 12H-2λ⁵,10λ⁵-5,8-methanofuro[3,2-1][1, 3,6,9,11,2,10]pentaoxadiphosphacyclotetradecine-2,10-dione. The present invention also encompasses intermediates useful in the disclosed synthetic processes and the methods of their preparation.

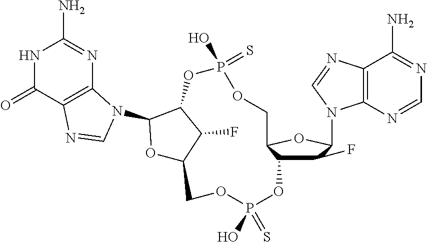 [SMILES: Nc1nc2c(ncn2[C@@H]2O[C@@H]3CO[P@@](O)(=S)O[C@H]4[C@H](F)[C@H](n5cnc6c(N)ncnc65)O[C@@H]4CO[P@@](O)(=S)O[C@@H]2[C@@H]3F)c(=O)[nH]1]

### FIELD OF THE INVENTION

The present invention relates to efficient synthetic processes useful in the preparation of fluorinated cyclic dinucleotides, such as [P(R)]-2′-deoxy-2′-fluoro-5′-O—[(R)-hydroxymercapto-phosphinyl]-P-thio-β-D-arabino-adenylyl-(3′→5′)-3′-deoxy-3′-fluoroguanosine cyclic nucleotide, which is also known as (2R,5R,7R,8S,10R,12aR,14R,15S,15aR,16R)-7-(2-amino-6-oxo-1,6-dihydro-9H-purin-9-yl)-14-(6-amino-9H-purin-9-yl)-15,16-difluoro-2,10-bis(sulfanyl) octahydro-2H,10H,12H-2λ⁵, 10λ⁵-5,8-methanofuro[3,2-1][1,3,6,9,11,2,10]pentaoxadiphospha-cyclotetradecine-2,10-dione. Such fluorinated cyclic dinucleotides may be useful as biologically active compounds.

### SEQUENCE LISTING

The instant application contains a Sequence Listing, which has been submitted electronically in ASCII format and is hereby incorporated by reference in its entirety. Said ASCII copy, created on Feb. 8, 2022, is named 25216WOPCT-SEQTXT-8FEB2022.txt and is 89,469 bytes in size.

### BACKGROUND OF THE INVENTION

The synthesis of complex nucleotides and nucleosides continue to challenge the synthetic community, notwithstanding many years of attempts motivated by their medicinal importance. J. J. Fox, et al., Chapter 10: Antiviral Activities of 2′-Fluorinated Arabinosyl-Pyrimidine Nucleosides, in FLUORINATED CARBOHYDRATES —CHEMICAL AND BIOCHEMICAL ASPECTS, ACS SYMPOSIUM SERIES, Vol. 374, 176-190 (1988).

The addition of fluorine stereocenters can greatly enhance the desired biological activity of nucleosides. For example, the introduction of fluorine to the 3′ position of the sugar moiety has been found to lead to more potent antiviral agents. However, the introduction of such stereocenters adds another level of difficulty beyond the already challenging synthesis. See, e.g., Piet Herdewijn et al., 31(10) J. MED. CHEM. 2040-2048 (1988); Jan Balzarini et al., 37(14) BIOCHEM. PHARMACOL. 2847-2856 (1988); George W. J. Fleet et al., 44(2) TETRAHEDRON 625-636 (1988); E. Matthes et al., 153(2) BIOCHEM. BIOPHYS. RES. COMMUN. 825-831 (1988); and Piet Herdewijn et al., 30(8) J. MED. CHEM. 1270-1278 (1987).

Cyclic dinucleotide (CDNs) thiophosphates are particularly challenging synthetic targets, and CDNs have become the focus of intense medicinal interest as immuno-oncology therapeutics due to dramatic improvement of both cellular uptake and metabolic stability of promising CDN analogs. However, the simple replacement of one atom from oxygen to sulfur (known as thio effect) has dramatically increased the synthetic challenges given the need to control two additional thiophosphoro stereogenic chiral centers during both activation and coupling steps. In general, over eight steps are required from the parent nucleoside to prepare a single CDN as a mixture of all four possible diastereoisomers with the aid of protecting groups and pre-installation of the activation groups in each nucleoside. See, e.g., Barbara Gaffney et al., 12(14) ORG. LETT. 3269-3271 (2010); Carlo Battistini et al., 49(5) TETRAHEDRON 1115-1132 (1993); Piotr Guga et al., 62(11) TETRAHEDRON 2698-2704 (2006); Hongbin Yan et al.; 18(20) BIOORG. MED. CHEM. LETT. 5631-5634 (2008); J. Zhao et al., NUCLEOSIDES NUCLEOTIDES NUCLEIC ACIDS 28, 352-378 (2009); Thierry Lioux et al., 59(22) J. MED. CHEM. 10253-10267 (2016).

Early approaches suffered from poor yields and required manipulation of protecting groups at several steps, and labor-intensive high-performance liquid chromatography (HPLC) purification is typically required to obtain a single CDN diastereomer. In light of the unmet medical needs in oncology and the significant limitations of current methods, there remains a need for improved syntheses of thiophosphoro cyclic dinucleotides, and in particular fluorinated thiophosphoro cyclic dinucleotides, such as [P(R)]-2′-deoxy-2′-fluoro-5′-O—[(R)-hydroxymercaptophosphinyl]-P-thio-β-D-arabino-adenylyl-(3′→5′)-3′-deoxy-3′-fluoroguanosine cyclic nucleotide.

### SUMMARY OF THE INVENTION

The present disclosure relates to processes useful in the synthesis of thiophosphoro cyclic dinucleotides, particularly fluorinated thiophosphoro cyclic dinucleotides, such as [P(R)]-2′-deoxy-2′-fluoro-5′-O—[(R)-hydroxymercaptophosphinyl]-P-thio-β-D-arabino-adenylyl-(3′→5′)-3′-deoxy-3′-fluoroguanosine cyclic nucleotide, which is also known as (2R,5R,7R,8S,10R,12aR, 14R,15S,15aR,16R)-7-(2-amino-6-oxo-1,6-dihydro-9H-purin-9-yl)-14-(6-amino-9H-purin-9-yl)-15,16-difluoro-2,10-bis(sulfanyl)octahydro-2H,10H,12H-2λ⁵,10λ⁵-5, 8-methanofuro[3,2-1] [1,3,6, 9,11,2,10] pentaoxadiphosphacyclotetradecine-2,10-dione.

The present disclosure also encompasses chemical processes that afford intermediates useful in the production of such fluorinated thiophosphoro cyclic dinucleotides. The chemical processes of the present disclosure afford advantages over previously known procedures and include a more efficient route to fluorinated thiophosphoro cyclic dinucleotides starting from readily available starting materials such as guanosine or xylose. The synthetic strategy uses an evolved cGAS enzyme, along with evolved kinase enzymes, to prepare a fluorinated cyclic dinucleotide by a process in which two thiotriphosphates are prepared from corresponding thio-monophosphates with high diastereoselectivity, followed by the addition of cGAS catalyst and metal co-factors to afford the corresponding CDN. These processes, described in further detail herein, provide a shorter, two step, synthetic route than previous synthetic routes, which generates no detectable diastereomeric impurities and a product that is directly isolatable by pH-swing crystallization.

Other embodiments, aspects, and features of the present disclosure are either further described in or will be apparent from the ensuing description, examples, and claims.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates.

That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

As used herein, including the appended claims, the singular forms of words, such as “a,” “can,” and “the,” include their corresponding plural references unless the context clearly dictates otherwise. In particular, “a,” “an,” and “the” item each include a single item selected from a list as well as mixtures of two or more items selected from the list.

As used herein, the terms “at least one” item or “one or more” item each include a single item selected from the list as well as mixtures of two or more items selected from the list. For example, “at least one cGAS type enzyme” (alternatively referred to as “cGAS type enzymes”) refers to a single cGAS type enzyme as well as to mixtures of two or more different cGAS type enzymes. Similarly, the terms “at least two” items and “two or more” items each include mixtures of two items selected from the list as well as mixtures of three or more items selected from the list. “Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Throughout this specification and claims, the word “comprise,” or variations such as “comprises” or “comprising” will be understood to imply the inclusion of a stated element or group of elements but not the exclusion of any other element or group of elements. In the case of integers, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between.

All ranges also are intended to include all included sub-ranges, although not necessarily explicitly set forth. For example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, and ±10% and their numerical equivalents. “About” when used to modify a numerically defined parameter means that the parameter may vary by as much as 10% below or above the stated numerical value; where appropriate, the stated parameter may be rounded to the nearest whole number. For example, an amount of about 5 mg may vary between 4.5 mg and 5.5 mg. In addition, the term “or,” as used herein, denotes alternatives that may, where appropriate, be combined; that is, the term “or” includes each listed alternative separately as well as their combination.

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl, and neohexyl. In one embodiment, an alkyl group is linear. In another embodiment, an alkyl group is branched.

The terms “halogen” and “halo,” as used herein, means —F (fluorine), —Cl (chlorine), —Br (bromine), or —I (iodine).

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group has from 1 to 3 carbon atoms. In another embodiment, a haloalkyl group is substituted with from 1 to 3 halogen atoms. Non-limiting examples of haloalkyl groups include —CH₂F, —CHF₂, and —CF₃. The term “C₁-C₄ haloalkyl” refers to a haloalkyl group having from 1 to 4 carbon atoms.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy, and tert-butoxy. An alkoxy group is bonded via its oxygen atom to the rest of the molecule.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. Non-limiting examples of aryl groups include phenyl and naphthyl.

When a functional group in a compound is termed “protected,” that functional group is in modified form to preclude undesired side reactions at the protected site when the compound is subjected to a reaction. The term “PG”, as used herein, refers to a protecting group. Those skilled in the art will readily envisage protecting groups suitable for use in compounds and processes according to the disclosure. Suitable protecting groups will be recognized by those of ordinary skill in the art as well as by reference to standard textbooks such as, for example, GREEN'S PROTECTIVE GROUPS IN ORGANIC SYNTHESIS (5^{th} ed., Peter G. M. Wuts ed, 2014). Protecting groups suitable for use in the processes disclosed herein include acid-labile protecting groups. Non-limiting examples of PG suitable for use herein include —S(O)₂R⁸, —C(O)OR⁸, —C(O)R⁸, —CH₂OCH₂CH₂SiR⁸, and —CH₂R₈, wherein R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I.

The term “substituted” means that one or more hydrogens on the atoms of the designated moiety are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

When any substituent or variable occurs more than one time in any compound, its definition on each occurrence is independent of its definition at every other occurrence, unless otherwise indicated. For example, description of radicals that include the expression “—N(C₁-C₃ alkyl)₂” means —N(CH₃)(CH₂CH₃), —N(CH₃)(CH₂CH₂CH₃), and —N(CH₂CH₃)(CH₂CH₂CH₃), as well as —N(CH₃)₂, —N(CH₂CH₃)₂, and —N(CH₂CH₂CH₃)₂.

It should also be noted that any carbon or heteroatom with unsatisfied valences in the text, schemes, examples, and tables herein is assumed to have sufficient hydrogen atom(s) to satisfy the valences. Any one or more of these hydrogen atoms can be deuterium.

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²³I respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half-lives (T_{1/2}>1 day), can generally be prepared by following procedures analogous to those disclosed in the Schemes and/or in the Examples herein below, by substituting an appropriate isotopically labeled reagent for a non-isotopically labeled reagent.

Compounds herein may contain one or more stereogenic centers and can occur as racemates, racemic mixtures, single enantiomers, diastereomeric mixtures, and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates, and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the S or R configuration as defined by the IUPAC 1974 Recommendations.

The present disclosure further includes compounds and synthetic intermediates in all their isolated forms. For example, the above-identified compounds are intended to encompass all forms of the compounds such as, any solvates, hydrates, stereoisomers, and tautomers thereof.

Those skilled in the art will recognize that chiral compounds, and in particular, in sugars, can be drawn in a number of different ways that are equivalent. Those skilled in the art will further recognize that the identity and regiochemical position of the substituents on ribose can vary widely and that the same principles of stereochemical equivalence apply regardless of substituent. Non-limiting examples of such equivalence include those exemplified below.

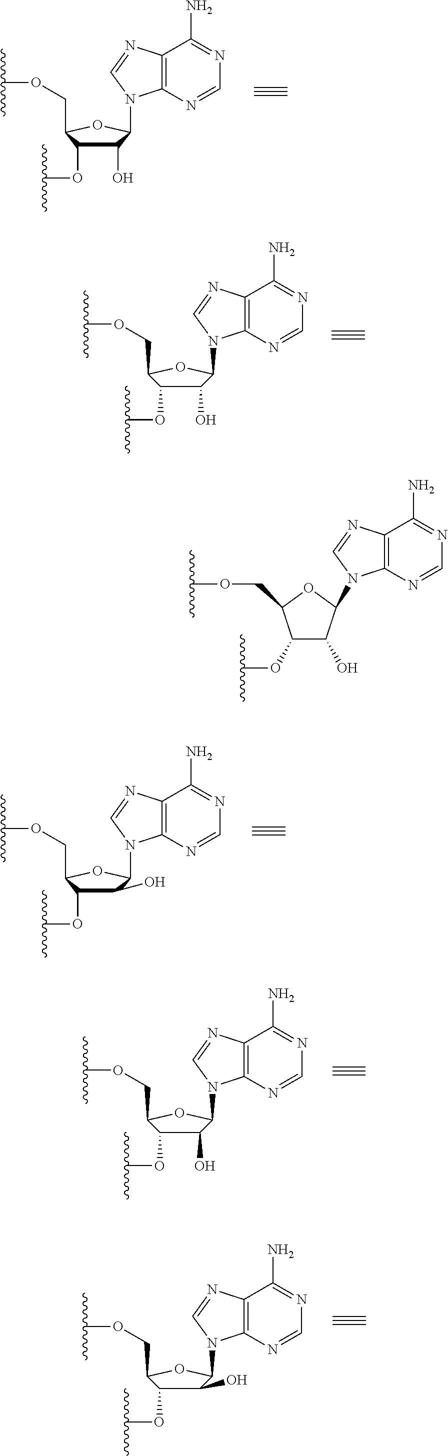

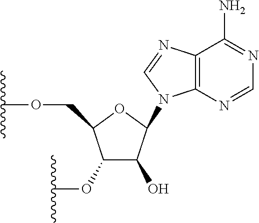 [SMILES: *OC[C@H]1O[C@@H](n2cnc3c(N)ncnc32)[C@@H](O)[C@@H]1O*]

Similarly, those skilled in the art will recognize that certain compounds, and in particular compounds containing certain heteroatoms and double or triple bonds, can be tautomers, structural isomers that readily interconvert. Thus, tautomeric compounds can be drawn in a number of different ways that are equivalent. Non-limiting examples of such tautomers include those exemplified below.

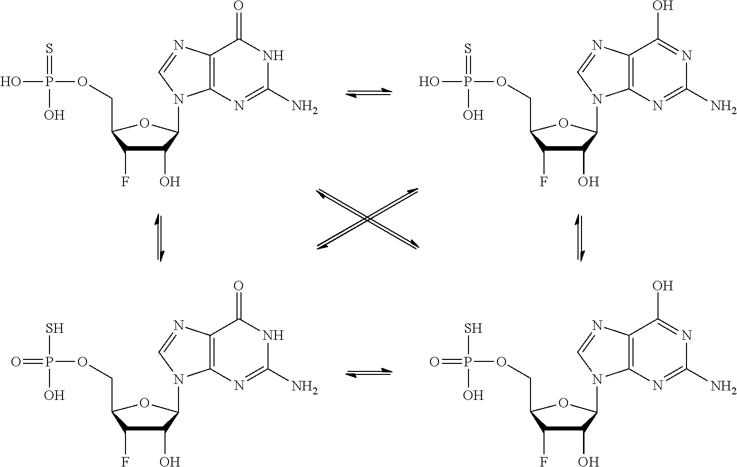

Compounds can form salts that are also within the scope of this disclosure. Reference to a compound herein is understood to include reference to salts thereof, unless otherwise indicated. The term “salt(s),” as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,), and the like. Additionally, acids that are generally considered suitable for the formation of pharmaceutically useful salts from basic pharmaceutical compounds are discussed, for example, by P. Stahl et al., Camille G. (eds.) HANDBOOK OF PHARMACEUTICAL SALTS: PROPERTIES, SELECTION AND USE (2002) Zurich: Wiley-VCH; S. Berge et al., J. Pharm. Sci. (1977) 66(1) 1-19; P. Gould, International J. of Pharmaceutics (1986) 33 201-217; Anderson et al., THE PRACTICE OF MEDICINAL CHEMISTRY (1996), Academic Press, New York; and in THE ORANGE BOOK (Food & Drug Administration, Washington, D.C.). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine, and the like. Basic nitrogen-containing groups may be quartemized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides, and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

One or more compounds herein may exist in unsolvated as well as solvated forms with pharmaceutically acceptable solvents, such as water, ethanol, and the like, and this disclosure is intended to embrace both solvated and unsolvated forms. “Solvate” means a physical association of a compound with one or more solvent molecules. This physical association involves varying degrees of ionic and covalent bonding, including hydrogen bonding. In certain instances, the solvate will be capable of isolation, for example when one or more solvent molecules are incorporated in the crystal lattice of a crystalline solid. “Solvate” encompasses both solution-phase and isolatable solvates. Non-limiting examples of suitable solvates include ethanolates, methanolates, and the like. “Hydrate” is a solvate in which the solvent molecule is H₂O.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristilation, ubiquitination, etc.). Included within this definition are D- and L-amino acids, and mixtures of D- and L-amino acids, as well as polymers comprising D- and L-amino acids, and mixtures of D- and L-amino acids.

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of enzymes, identifies the originating enzyme, and/or the gene encoding such enzyme, upon which the enzyme was based. For example, the cyclic GMP-AMP synthase (cGAS) type enzyme of SEQ ID NO: 13 was obtained by artificially evolving, over multiple generations the gene encoding the wild-type cGAS type enzyme of SEQ ID NO: 1. Thus, this evolved cGAS type enzyme is “derived from” the wild-type cGAS type enzyme of SEQ ID NO: 1.

“Hydrophilic amino acid or residue” refers to an amino acid or residue having a side chain exhibiting a hydrophobicity of less than zero according to the normalized consensus hydrophobicity scale of Eisenberg et al., 1984, J. MOL. BIOL. 179:125-142. Genetically encoded hydrophilic amino acids include L-Thr (T), L-Ser (S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S), and L-Thr (T). “Hydrophobic amino acid or residue” refers to an amino acid or residue having a side chain exhibiting a hydrophobicity of greater than zero according to the normalized consensus hydrophobicity scale of Eisenberg et al., 1984, J. MOL. BIOL. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H).

Histidine has a constrained geometry because it has a relatively small imidazole ring. Proline has a constrained geometry because it also has a five-membered ring.

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the a-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T), and L-Asp (D).

“Hydroxyl-containing amino acid or residue” refers to an amino acid containing a hydroxyl (—OH) moiety. Genetically-encoded hydroxyl-containing amino acids include L-Ser (S) L-Thr (T), and L-Tyr (Y).

As used herein, “polynucleotide” and “nucleic acid” refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

As used herein, “nucleoside” refers to glycosylamines comprising a nucleobase (i.e., a nitrogenous base), and a 5-carbon sugar (e.g., ribose or deoxyribose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups. In some embodiments, nucleosides can be phosphorylated by kinases to produce nucleotides.

As used herein, “nucleoside diphosphate” refers to glycosylamines comprising a nucleobase (i.e., a nitrogenous base), a 5-carbon sugar (e.g., ribose or deoxyribose), and a diphosphate (i.e., pyrophosphate) moiety. In some embodiments herein, “nucleoside diphosphate” is abbreviated as “NDP.” Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). The terms “nucleoside” and “nucleotide” may be used interchangeably in some contexts.

As used herein, “nucleoside triphosphate” refers to glycosylamines comprising a nucleobase (i.e., a nitrogenous base), a 5-carbon sugar (e.g., ribose or deoxyribose), and a triphosphate moiety. In some embodiments herein, “nucleoside triphosphate” is abbreviated as “NTP.” Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). The terms “nucleoside” and “nucleotide” may be used interchangeably in some contexts.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the wild-type enzyme, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.5 times the enzymatic activity of the corresponding wild-type enzyme, to as much as 2 times. 5 times, 10 times, 20 times, 25 times, 50 times, 75 times, 100 times, 150 times, 200 times, 500 times, 1000 times, 3000 times, 5000 times, 7000 times or more enzymatic activity than the naturally occurring enzyme or another enzyme from which the polypeptides were derived. In specific embodiments, the enzyme exhibits improved enzymatic activity in the range of 150 to 3000 times, 3000 to 7000 times, or more than 7000 times greater than that of the parent enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{ca}/Kₘ, is generally about 108 to 109 (M⁻¹s⁻¹). Hence, any improvements in the enzyme activity will have an upper limit related to the diffusion rate of the substrates acted on by the enzyme. Enzyme activity can be measured by any one of standard assays used for measuring kinase activity, or via a coupled assay with an nucleoside phosphorylase enzyme which is capable of catalyzing reaction between the polypeptide product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, “analogues” means polypeptides that contain one or more non-naturally occurring amino acid residues including, but not limited, to homoarginine, ornithine, and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB).

The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. MOL. BIOL. 215: 403-410; and Altschul et al., 1977, NUCLEIC ACIDS RES. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. SCI. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, ADV. APPL. MATH. 2:482, by the homology alignment algorithm of Needleman and Wunsch, 1970, J. MOL. BIOL. 48:443, by the search for similarity method of Pearson and Lipman, 1988, PROC. NATL. ACAD. SCI. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to”, or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (EE) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereomeric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to a polypeptide that maintains similar activity (more than 60% to 80% for example) after exposure to elevated temperatures (e.g., 40-80° C.) for a period of time (e.g., 0.5-24 h) compared to the untreated enzyme.

“Solvent stable” refers to a polypeptide that maintains similar activity (more than e.g., 60% to 80%) after exposure to varying concentrations (e.g., 5-99%) of solvent (isopropyl alcohol, tetrahydrofuran, 2-methyltetrahydrofuran, acetone, toluene, butylacetate, methyl tert-butylether, etc.) for a period of time (e.g., 0.5-24 h) compared to the untreated enzyme.

“pH stable” refers to a polypeptide that maintains similar activity (more than e.g., 60% to 80%) after exposure to high or low pH (e.g., 4.5 to 6 or 8 to 12) for a period of time (e.g., 0.5-24 h) compared to the untreated enzyme.

“Thermo- and solvent stable” refers to a polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

Exemplary methods and materials are described herein, although methods and materials similar or equivalent to those described herein can also be used in the practice or testing of the present disclosure. The materials, methods, and examples are illustrative only and not intended to be limiting.

### ABBREVIATIONS

2,4,6-collidine 2,4,6-Trimethylpyridine
2,6-lutidine 2,6-Dimethylpyridine
2′-F-thio-AMP (0-{[(2R,3R,4S,5R)-5-(6-amino-9H-purin-9-yl)-4-fluoro-3-hydroxyoxolan-2-yl]methyl} 0,0-dihydrogen phosphorothioate, also known as 2′-fluoro-thio-adenosine monophosphate
2′-FA (2R,3R,4R,5R)-5-(6-amino-9H-purin-9-yl)-4-fluoro-2-(hydroxymethyl)tetrahydrofuran-3-ol
2-Me-THF 2-Methyltetrahydrofuran
2-TBS 2-tert-butyl(((2R,3S)-3-((tert-butyldimethylsilyl)oxy)-2,3-dihydrofuran2-yl)methoxy)dimethylsilane
3′-F-thio-GMP (2S,3R,4S,5R)-5-(2-amino-6-oxo-1,6-dihydro-9H-purin-9-yl)-3-fluoro-4-hydroxy-2-(mercaptomethyl)tetrahydrofuran-3-yl dihydrogen phosphate, also known as 3′-fluoro-thio-guanosine monophosphate
3′-FG 9-((2R,3S,4S,5R)-4-fluoro-3-hydroxy-5-(hydroxymethyl)tetrahydrofuran-2-yl)-2-(isobutylamino)-1,9-dihydro-6H-purin-6-one
3-TBS N-((2S,3S,4R,5R)-4-((tert-butyldimethylsilyl)oxy)-5-(((tert-butyldimethylsilyl)oxy)methyl)-3-fluorotetrahydrofuran-2-yl)-N-(phenylsulfonyl) benzenesulfonamide
Ac Acetyl
Acac, acac Acetyl acetonate anion
ACN, MeCN Acetonitrile
AcOH, HOAc Acetic acid
AcP—K/K Dipotassium acetylphosphate
AcP—Li/K Lithium potassium acetylphosphate
AcP—Li/Li Dilithium acetylphosphate
AcP—Na/Na Disodium acetylphosphate
AcP—NH₄/NH₄ Diammonium acetylphosphate
AMP Adenosine monophosphate
aq Aqueous
ATP Adenosine triphosphate
BAST bis(2-Methoxyethyl)aminosulfur trifluoride
bis-Tris 2-[bis(2-hydroxyethyl)amino]-2-(hydroxymethyl)propane-1,3-diol
Bn Benzyl
Boc or BOC Butoxycarbonyl
BSA Bistrimethylsilyl acetamide
BSTFA Bistrimethylsilyl trifluoroacetamide, also referred to as trimethylsilyl 2,2,2-trifluoro-N-(trimethylsilyl)acetimidate
Bu Butyl
Bz Benzoyl
CDN Cyclic dinucleotide
CFE Cell-free extract
cGAS Cyclic GMP-AMP synthase
conc. Concentrated
CPME Cyclopentylmethyl ether
DABCO 1,3-Diazabicyclo[2.2.2]octane
DAST Diethylaminosulfur trifluoride
DBSI N,N-Dibenzenesulfonimide
DBU 1,8-Diazabicyclo[5.4.0]undec-7-ene
DCA Dichloroacetic acid
DCM, CH₂Cl₂ Dichloromethane
DI water Deionized water
DMAc Dimethylacetamide
DMAP 4-Dimethylaminopyridine
DME, Glyme Dimethoxyethane
DMF N,N-Dimethylformamide
DMPU N,N′-Dimethylpropyleneurea
DMSO Dimethyl sulfoxide
EDTA Ethylene diamine tetraacetic acid
eq Equivalents
EtOAc Ethyl acetate
EtOH Ethanol
g Grams
g/l Grams per liter
GMP Guanosine monophophate
h Hour
H₂O Water
HDMS Hexamethyldisilazane
HEPES 2-[4-(2-hydroxyethyl)piperazin-1-yl]ethanesulfonic acid
HFIP Hexafluoro-2-propanol
[HN(n-oct)₃]₂[SO₄] Tri-n-octylammonium hydrogen sulfate
HPLC High-performance liquid chromatography
IDA Iminodiacetic acid
IPA, i-PrOH Isopropyl alcohol
IPAc Isopropyl acetate
IPTG Isopropyl [3-D-1-thiogalactopyranoside
KF Potassium fluoride
M Molar, moles per liter
MeOH Methanol
MES 2-Morpholin-4-ylethanesulfonic acid
mg Milligrams
MIBK Methyl isobutyl ketone
min Minute(s)
ML Mother liquor
mL or ml Milliliters
mM Millimole per liter
mmol Millimoles
MPA 1-Methoxy-2-propyl acetate
Ms Methanesulfonyl
MTBE Methyl tert-butyl ether, methyl tertiary butyl ether
NADPH Nicotinamide adenine dinucleotide phosphate
n-BuLi n-Butyllithium
NFSI N-fluorobenzenesulfonimide
Ni-NTA Nickel nitrilotriacetic acid
NMI 1-Methylimidazole
NMP N-Methyl-2-pyrrolidone
NTA Nitrilotriacetic acid
OD Optical density
PBS Phosphate-buffered saline
PG Protecting group
PIV or Piv Pivalate, 2,2-Dimethylpropanoate
PSePI N,N-bis(diphenylselenophosphoryl)amide
PTPI N,N-bis(diphenylthiophosphoryl)amide
Py Pyridine
RPM, rpm Revolutions per minute
RT Room temperature, approximately 25° C.
SDS Sodium dodecyl sulfate
STAB Sodium triacetoxyborohydride
TAPSO 3-[[1,3-Dihydroxy-2-(hydroxymethyl)propan-2-yl]amino]-2-hydroxypropane-1-sulfonic acid
TBS tert-Butyldimethylsilyl
TED Tris carboxymethyl ethylene diamine
TES 2-[(2-Hydroxy-1,1-bis(hydroxymethyl)ethyl)amino] ethanesulfonic acid, I-[Tris(hydroxymethyl)methyl]-2-aminoethanesulfonic acid, TES free acid
Tf, OTf Trifluoromethanesolfonate, CF₃SO₃—, or triflate
TFA Trifluoroacetic acid
TFE 2,2,2-Trifluoroethanol
TGDE, Tetraglyme Tetraethylene glycol dimethyl ether
THF Tetrahydrofuran
Tris 2-Amino-2-(hydroxymethyl)propane-1,3-diol
Trityl Triphenylmethyl
Ts, OTs para-Toluenesulfonyl or tosyl
UPLC Ultra Performance Liquid Chromatography
Vol. or vol. Volumes
wt % Percent by weight or weight percent
XG Xyloguanosine
μL or μl Microliters

The present disclosure provides a process for preparing compounds of Formula (I) and pharmaceutically acceptable salts, hydrates, and solvates thereof:

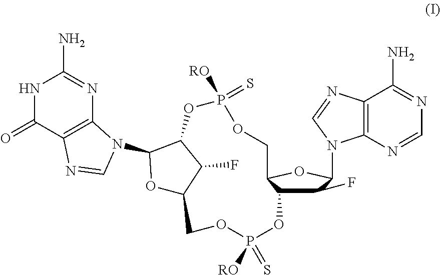

wherein each R is a cation independently selected from the group consisting of H⁺, Na⁺, K⁺, Mg⁺⁺, Co⁺⁺, Zn⁺⁺, and NH₄⁺. The compounds of Formula (I), and pharmaceutically acceptable salts, hydrates, and solvates thereof, may alternatively be drawn as:

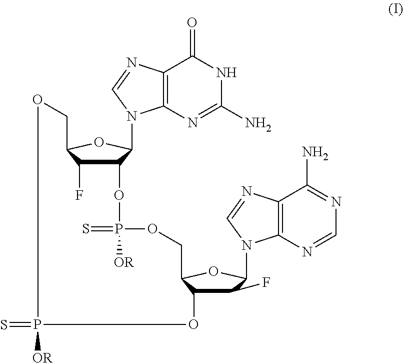

wherein R is as defined above. In specific embodiments, the disclosure provides a process for preparing a compound of Formula (Ia), and pharmaceutically acceptable salts, hydrates, and solvates thereof:

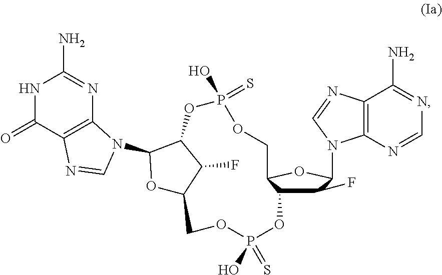 [SMILES: Nc1nc2c(ncn2[C@@H]2O[C@@H]3CO[P@@](O)(=S)O[C@H]4[C@H](F)[C@H](n5cnc6c(N)ncnc65)O[C@@H]4CO[P@@](O)(=S)O[C@@H]2[C@@H]3F)c(=O)[nH]1]

which may also be drawn as:

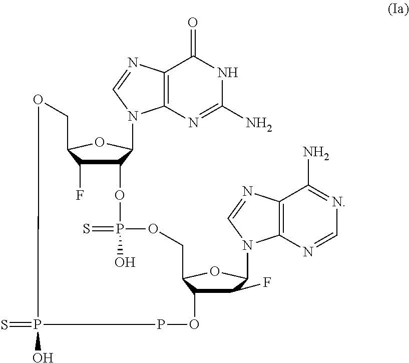

In embodiments, the compound of Formula (Ia) is prepared from (0-{[(2R,3R,4S,5R)-5-(6-amino-9H-purin-9-yl)-4-fluoro-3-hydroxyoxolan-2-yl]methyl} 0,0-dihydrogen phosphorothioate (also known as 2′-fluoro-thio-adenosine monophosphate or 2′-F-thio-AMP) and (2S,3R,4S,5R)-5-(2-amino-6-oxo-1,6-dihydro-9H-purin-9-yl)-3-fluoro-4-hydroxy-2-(mercaptomethyl)tetrahydrofuran-3-yl dihydrogen phosphate (also known as 3′-fluoro-thio-guanosine monophosphate or 3′-F-thio-GMP) as starting materials. (0-{[(2R,3R,4S,5R)-5-(6-amino-9H-purin-9-yl)-4-fluoro-3-hydroxyoxolan-2-yl]methyl} 0,0-dihydrogen phosphorothioate (also known as 2′-fluoro-thio-adenosine monophosphate or 2′-F-thio-AMP) may be prepared from processes including those disclosed in U.S. Provisional Patent Application No. 63/065,732, filed on Aug. 14, 2020, and PCT International Patent Application Number PCT/US2021/045465, filed on Aug. 11, 2021, which published as WO2022/035917 on Feb. 17, 2022. (2S,3R,4S,5R)-5-(2-amino-6-oxo-1,6-dihydro-9H-purin-9-yl)-3-fluoro-4-hydroxy-2-(mercaptomethyl) tetrahydrofuran-3-yl dihydrogen phosphate (also known as 3′-fluoro-thio-guanosine monophosphate or 3′-F-thio-GMP) may be prepared from processes including those disclosed in U.S. Provisional Patent Application No. 63/028,741, filed on May 22, 2020, and PCT International Patent Application Number PCT/US2021/033286, filed on May 20, 2021, which published as WO2021/236859 on Nov. 25, 2021.

A first embodiment relates to a process for preparing a compound of Formula (Ia), which comprises reacting a compound of Formula (I-1) with a compound of Formula (I-2), in the presence of at least one cyclic GMP-AMP synthase (cGAS) type enzyme.

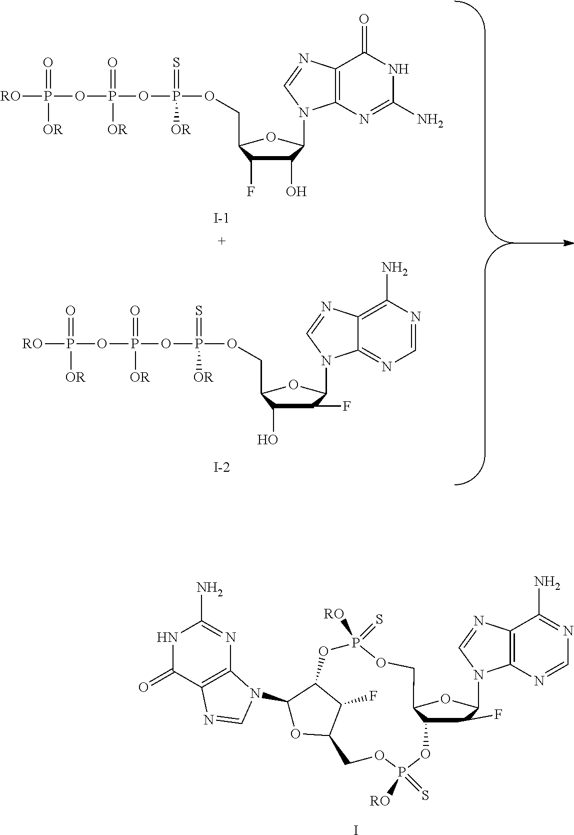

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In a first aspect of the first embodiment, the compound of Formula (I-1) and compound of Formula (I-2) are provided in a ratio of from about 10:1, from about 7:1, from about 5:1, from about 4:1, from about 2:1, from about 1:1, from about 1:2, from about 1:4, from about 1:5, from about 1:7, or from about 1:10.

In a second aspect of the first embodiment, the at least one cyclic GMP-AMP synthase (cGAS) type enzyme is selected from the group consisting of wild-type cGAS type enzymes and cGAS type enzymes that are the product of directed evolution from a wild-type cGAS type enzyme. In specific instances, the at least one cGAS type enzyme is the wild-type cGAS type enzyme having the amino acid sequence that is SEQ ID NO:1.

(SEQ ID NO: 1)
MPSGDWREAVRPRPSSPAVGQEGAGFVSSGEERCVEEGVRPPEAEGGKPR
RGSRRPVAAAASSSGGPRLREVLSRLSLGRQDVSEASGLVNQVVSQLIQA
IRSQEGSFGSIERLGAGSYYEHVKISEPNEFDIMLVMPVSRLQLDECDDT
GAYYYLTFKRNPKEKHLFKFLDEDGKLSAFKMLQALRDIIKREVKNIKNA
EVTVKRRKAGSPAITLQIKNPPAEISVDIILTLEVQQNWPPSTQDGLKIE
QWLGRKVRGQFRNKSLYLVAKQNKREKVLRGNTWRLSFSHIEKDMLNNHG
SSKTCCESDGLKCCRKGCLKLLKYLLEQLKMKYTKQLEKFCSYHVKTAFF
HSCVMWPNDTDWHLGDLDYCFQKYLGYFLDCLQKSELPHFFIPQYNLLSL
EDKASNDFLSRQINYQLNNRFPIFQERY

In a first instance of this second aspect, the at least one cGAS type enzyme has the amino acid sequence that is SEQ ID NO: 2.

(SEQ ID NO: 2)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNAGSYYEHVKISEPNEFDIMLVMPVSRLQLDECDDTGA
FYYLTFKRNPKEKHLFKFLDEDGKLSAFKMLQALRDIIKREVKNIKNAEV
TVKRRKAGSPAITLQIKNPPAEISVDIILTLEVQQNWPPSTQDGLKIEQW
LGRKVRGQFRNKSLYLVAKQNKREKVLRGNTWRLSFSHIEKDMLNNHGSS
KTCCESDGLKCCRKGCLKLLKYLLEQLKMKYTKQLEKFSSYHVKTAFFHS
CVMWPNDTDWHLGDLDYCFQKYLGYFLDCLQKSELPHFFIPQYNLLSLED
KASNDFLSRQINYQLNNRFPIFQERY

In a second instance of this second aspect, the at least one cGAS type enzyme has the amino acid sequence that is SEQ ID NO: 3.

(SEQ ID NO: 3)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNPKEKHLFKFLDEDGKLSAFKMLQALRDIIKREVKNIKNAEV
TVKRKKAGSPAITLQIKNPPAEISVDIILTLEVQQNWPPSTQDGLKIEQW
LGRKVRGQFRNKSLYLVAKQNKREKVLRGNTWRISFSHIEKDMLNNHGSS
KTCCESDGLKCCRKGCLKLLKYLLEQLKMKYTKQLEKFSSYHVKTAFFHS
CVMWPNDTDWHLGDLDYCFQKYLGYFLDCLQKSELPHFFIPQYNLLSLED
KASNDFLSRQINYELNNRFPIFQERY

In a third instance of this second aspect, the at least one cGAS type enzyme has the amino acid sequence that is SEQ ID NO: 4.

(SEQ ID NO: 4)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNPKEKHLFKFLDEDGKLSAFKMLQALRDIIKREVKNIKNAEV
TVKRKKAGSPAITLQIKNPPAEISVDIILTLEVQQSWPPSTQDGLKIEQW
LGRKVRGQFRNKSLYLVAKQNKREKVLRGNTWRISFSHIEKDMLNNHGSS
KTCCESDGLKCCRKGCLKLLKYLLEQLKMKYTKQLEKFSSYEVKTAFFHS
CVMWPNDTDWHLGDLDYCFQKYLGYFLDCLQKSELPHFFIPQYNLLSLED
KASNDFLSRQINYELNNRFPIFQERY

In a fourth instance of this second aspect, the at least one cGAS type enzyme has the amino acid sequence that is SEQ ID NO: 5.

(SEQ ID NO: 5)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNPKEKHLFKFLDEDGKLSAFKMLQALRDIIKREVKNIKNAEV
TVKRKKAGSPAITLQIKNPPAEISVDIILTLEVQQSWPPSTQDGLKIEQW
LGRKVRGQFRNKSLYLVAKQNKREKVLRGNTWRISFSHIEKDMLNNHGSS
KTCCESDGLKCCRKGCYKLLKYLLEQLKMKYTKQLEKFSSYEVKTAFFHS
CVMWPNDTDWHLGDLDYCFQKYLGYFLDCLQKSELPHFFIPQYNLLSLED
KASNDFLSRQINYELNNRFPIFQERY

In a fifth instance of this second aspect, the at least one cGAS type enzyme has the amino acid sequence that is SEQ ID NO: 6.

(SEQ ID NO: 6)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNQKHKHLFKFLDEDGKLSAFKMLQALRDIIKREVKNIKNAEV
TVKRKKAGSPAITLQIKNPPAEISVDIILTLEVQQSWPPSTQDGLKIEKW
LGRKVRGQFRNKSLYLVAKQNKREKVLRGNTWRISFSHIEKDMLNNHGSS
KTCCESDGLKCCRKGCYKLLKYLLEQLKMKYPHQLEKFSSYEVKTAFFHS
CVMWPNDSDWHLGDLDYCFQKYLGYFLDCLQKSELPHFFIPQYNLLSLED
KASNDFLSRQINYELNNRFPIFQERY

In a sixth instance of this second aspect, the at least one cGAS type enzyme has the amino acid sequence that is SEQ ID NO: 7.

(SEQ ID NO: 7)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEVQQSWPPSTQDGLKIEKW
LGRKVRGQFRNKSLYLVAKQNKREKVLRGNTWRISFSHIEKDMLNNHGSS
KTCCESDGLKCCRKGCYKLLKYLLERLKMKYPHQLEKRSSYEVKTAFFHS
CVMWPNDSDWHLSDLDYCFQKYLGYFLDCLQKSELPHFFIPQYNLLSLED
KASNDFLSRQINYELNNRFPIFQERY

In a seventh instance of this second aspect, the at least one cGAS type enzyme has the amino acid sequence that is SEQ ID NO: 8.

(SEQ ID NO: 8)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNSGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEVQQSWPPSTQDGLKIEKW
LGRKVRGQFRNKSLYLVAKQNKREKVLRGNTWRISFSHIEKDMLNNHGSS
KTCCESDGLKCCRKGCYKLLKYLLERLKMKYPHQLEKRSSYEVKTAFFHS
CVMWPNDSDWHLSDLDYCFQKYLGYFLDCLQKSELPHFFIPQYNLLSLED
KASNDFLSRQINYELNNRFPIFQERY

In an eighth instance of this second aspect, the at least one cGAS type enzyme has the amino acid sequence that is SEQ ID NO: 9.

(SEQ ID NO: 9)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNSGSYYEHVKISEPNEFDIMLVMPVSRLQLDECDDTGA
FYYLTQKRNSKDKHLFKFLDEDGKLSAFKMLQALRDIIKREVKNIKNAEV
TVKRKKAGSPAITLQIKNPPAVISVDIILTLEVQQSWPPSTQDGLKIEKW
LGRKVRGQFRNKSLYLVAKQNKREKVLRGNTWRISFSHIEKDMLNNHGSS
KTCCESDGLKCCRKGCYKLLKYLLERLKMKYPHQLEKRSSYEVKTAFFHS
CVMWPNDSDWHLSDLDYCFQKYLGYFLDCLQKSELPHFFIPQYNLLSLED
KASNDFLSRQINYELNNRFPIFQERY

In a ninth instance of this second aspect, the at least one cGAS type enzyme has the amino acid sequence that is SEQ ID NO: 10.

(SEQ ID NO: 10)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEPQQSWPPSTQDGLKIEKW
LGRKVRGQFRNKSLYLVAKQNKREKVLRGNTWRISFSHIEKDMLNNHGSS
KTCCESDGLKCCRKGCYKLLKYLLERLKMKYPHQLEKRSSYEVKTAFFHS
CVMWPNDSDWHLSDLDYCFQKYLGYFLDCLQKSELPHFFIPQYNLLSLED
KASNDFLSRQINYELNNRFPIFQERY

In a tenth instance of this second aspect, the at least one cGAS type enzyme has the amino acid sequence that is SEQ ID NO: 11.

(SEQ ID NO: 11)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEPQQSWPPSTQDGLKIEKW
LGRKVRGQFRNKSLYLVAKQNKREKVLRGNTWRLSFSHIEKDMLNNHGSS
KTCCESDGLKCCRKGCYKLLKYLLERLKMKYPHQLEKRSSYEVKTAFFHS
CVMWPNDLDWHLSDLDYCFQKYLGYFLDCLQKSELPHFFIPQYNLLSLED
KASNDFLSRQINYELNNRFPIFQERY

In an eleventh instance of this second aspect, the at least one cGAS type enzyme has the amino acid sequence that is SEQ ID NO: 12.

(SEQ ID NO: 12)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEPQQSWPPSTQDGLKIEKW
LGRKVRGQFRNKSLYLVAKQNPREKVLRGNTWRLSFSHIEKDMLNNHGSS
KTCCESDGLKCCRKGCYKLLKYLLERLKMKYPHQLEKRSSYEVKTAFFHS
CVMWPNDLDWHLSDLDYCFQKYLGYFLDCLQKSELPHFFIPQYNLLSLED
KASNDFLSRQINYELNNRFPIFQERY

In a twelfth instance of this second aspect, the at least one cGAS type enzyme has the amino acid sequence that is SEQ ID NO: 13.

(SEQ ID NO: 13)
MHHHHHHGSDSEVNQEAKPEVKPEVKPETHINLKVSDGSSEIFFKIKKTT
PLRRLMEAFAKRQGKEMDSLTFLYDGIEIQADQTPEDLDMEDNDIIEAHR
EQIGGENLYFQGGGPRLREVLSRLSLGRQDVSEASGLVNQVVSQLIQAIR
SQEGSFGSIERLNTGSYYEHVKISEPNEFDIMLVMPVSRLQLDECDDTGA
FYYLTFKRNSKDKHLFKFLDEDGKLSAFKMLQALRDIIKREVKNIKNAEV
TVKRKKAGSPAITLQIKNPPAVISVDIILTLEPQQSWPPSTQDGLKIEKW
LGRKVRGQFRNKSLYLVAKQNPREKVLRGNTWRLSFSHIEKDMLNNHGSS
KTCCESDGLKCCRKGCYKLLKYLLEQLKMKYPKQLEKRSSYEVKTAFFHS
CVMWPNDTDWHLSDLDYCFQKYLGYFLDCLQKSELPHFFIPQYNLLSLED
KASNDFLSRQINYELNNRFPIFQERY

In a third aspect of the first embodiment, the at least one cGAS type enzyme is provided in an amount in a range of from about 1.0 percent by weight (wt %) to about 100.0 wt % with respect to the amount of the compound of Formula (I-1), such as an amount in a range of from about 10.0 wt % to about 50.0 wt %, or an amount in a range of from about 20.0 wt % to about 40.0 wt %.

In a fourth aspect of the first embodiment, the at least one cGAS type enzyme can be used as the whole cell lysate, a cGAS wet pellet, a purified cGAS wet pellet, a Co-treated cGAS wet pellet (i.e., cGAS enzyme treated with cobalt salt), or as a lyophilized powder.

In a fifth aspect of the first embodiment, the at least one cGAS type enzyme can be incubated in at least one Chaotropic Agent A including, but not limited to, sodium dodecyl sulfate (SDS), thiourea, guanidine HCl, phenol, phenyl acetyl sulfide, urea, KCl, MgCl₂, LiOAc, NaCl, and mixtures thereof. In instances of this fifth aspect of the first embodiment, the at least one chaotropic agent is MgCl₂.

In a first instance of the fifth aspect of the first embodiment, the at least one Chaotropic Agent A is provided in an amount in a range of from about 0.01M to 2M, such an amount in a range of from about 0.05M to 1M, or an amount in a range of from about 0.1M to 0.5M.

In a second instance of the fifth aspect of the first embodiment, the at least one Chaotropic Agent A is provided in a range of from about 5 to about 10 volumes with respect to the amount of the at least one cGAS type enzyme, such an amount in a range of from about 3 to 5 volumes, or an amount of about 1 volume.

In a third instance of the fifth aspect of the first embodiment, the at least one cGAS type enzyme is incubated in the at least one Chaotropic Agent A at a temperature range of 5° C. to 80° C., such as at a temperature in a range of from about 10° C. to about 50° C., or about 23° C.

In a fourth instance of the fifth aspect of the first embodiment, the at least one cGAS type enzyme is incubated in the at least one Chaotropic Agent A at a pH range of 4 to 14, such as at a pH in a range of from about 6 to about 10, or about 8.

In a sixth aspect of the first embodiment, the reacting further comprises reacting in the presence of at least one Metal Co-Factor A. In instances of this sixth aspect, the at least one Metal Co-Factor A is selected from the group consisting of KCl, MgCl₂, ZnSO₄, CoSO₄, CoF₂, Co(SCN)₂, CoBr₂, Co(NO₃)₂, CoCl₂, CoCO₃, Co(C₂O₄)₂, and Co(OH)₂, and mixtures thereof. In specific instances, the at least one Metal Co-Factor A is CoSO₄.

In a first instance of this sixth aspect, the at least one Metal Co-Factor A is provided in an amount in a range of from about 0.01M to 2M, such an amount in a range of from about 0.05M to 1M, or an amount in a range of from about 0.1M to 0.5M.

In a second instance of this sixth aspect, the at least one Metal Co-Factor A is provided in a range of from about 5 to about 10 volumes with respect to the amount of the at least one cGAS type enzyme, such an amount in a range of from about 3 to 5 volumes, or an amount of about 1 volume.

In a third instance of this sixth aspect, the at least one cGAS type enzyme is incubated with the at least one Metal Co-Factor A.

In a first occurrence of this third instance, the at least one cGAS type enzyme is incubated with the at least one Metal Co-Factor A at a temperature range of 5° C. to 80° C., such as at a temperature in a range of from about 10° C. to about 50° C., or about 23° C.

In a second occurrence of this third instance, the at least one cGAS type enzyme is incubated with the at least one Metal Co-Factor A at a pH range of 4 to 14, such as at a pH in a range of from about 6 to about 10, or pH of about 8.

In a third occurrence of this third instance, the at least one cGAS type enzyme is incubated with the at least one Metal Co-Factor A in the presence of at least one Base A from the group consisting of TES, KOH, and NaOH, and mixtures thereof. In more specific occurrences, the at least one Base A is KOH.

In a seventh aspect of the first embodiment, the reacting is conducted in the presence of at least one Inorganic Salt A. In instances of the seventh aspect, the at least one Inorganic Salt A is selected from the group consisting of CoSO₄, ZnSO₄, CoCl₂, Co(acac)₂, ZnF₂, ZnCl₂, MoCl₅, SbCl₅, CuCl, CuCl₂, CuBr, CuBr₂, CuF₂, CuOAc, CuSO₄, Fe(II)BF₄, V(O)(acac)₂, Pt(II)Cl₂, Ho(OTf)₃, Ni(II)Br, La(acac)₃, CeCl₃, KBF₄, MgCl₂, Zn(OTf)₂, ZnBr₂, CoBr₂, Zn(OAc)₂, Co(OAc)₂, Mg(OH)₂, hydrates of the aforementioned, and mixtures thereof. In specific instances of this seventh aspect, the at least one Inorganic Salt A is selected from the group consisting of CoSO₄, ZnSO₄, CoCl₂, ZnCl₂, Zn(OTf)₂, Zn(OAc)₂, and mixtures thereof. In specific instances of this seventh aspect, the at least one Inorganic Salt A is a mixture selected from the group consisting of CoCl₂ and ZnCl₂, CoCl₂ and Zn(OAc)₂, CoSO₄ and ZnCl₂, CoSO₄ and Zn(OTf)₂, CoSO₄ and Zn(OAc)₂, and CoSO₄ and Zn(OTf)₂. In a specific instance of this aspect, the Inorganic Salt A is selected from the group consisting of CoSO₄ and ZnSO₄, and mixtures thereof.

In a first instance of this seventh aspect, the at least one Inorganic Salt A is provided in an amount in a range of from about 0.1 to about 5.0 equivalents with respect to the amount of the compound of Formula (I-1).

In an eighth aspect of the first embodiment, the at least one cGAS type enzyme has been isolated. In instances of this aspect, a crude lysate containing the at least one cGAS type enzyme is subjected to centrifugation, and the pellet fraction is slurried and incubated with an aqueous solution of at least one Inorganic Salt B consisting of Na₂SO₄, (NH₄)₂SO₄, NaCl, KCl, K₂SO₄, hydrates of the aforementioned, and mixtures thereof. In particular instances, the volume of the aqueous solution of at least one Inorganic Salt B can range from 0.1 volumes to 5 volumes relative to the initial volume of crude lysate. In additional instances, the concentration of this solution can range from 0.1M to 1.5M. In instances of this aspect, the slurry is subjected to centrifugation following incubation with the aqueous solution of at least one Inorganic Salt B, and the liquid fraction containing cGAS type enzyme is retained.

In a first occurrence of the instances of the eighth aspect, the concentration of at least one Inorganic Salt B is reduced in liquid fraction containing cGAS type enzyme, which may be accomplished by a method selected from the group consisting of dialysis, tangential flow filtration, dilution with water, and dilution with a solution comprising at least one Inorganic Salt C, which is selected from the group consisting of CoSO₄, CoCl₂, ZnSO₄, ZnCl₂, MgSO₄, and MgCl₂, hydrates of the aforementioned, and mixtures thereof. In particular facets of this occurrence, the solution comprising at least one Inorganic Salt C includes the at least one Inorganic Salt C at a concentration of 0.01M to 0.1M. In specific facets of this occurrence, the at least one cGAS type enzyme will precipitate from the solution having reduced salt concentration and can then be isolated by centrifugation.

In a second occurrence of the instances of the eighth aspect, the at least one cGAS type enzyme is purified by a chromatographic technique. In particular facets of this occurrence, the chromatographic technique is selected from the group consisting of immobilized metal-affinity chromatography, ion-exchange chromatography, hydrophobic interaction chromatography, and size-exclusion chromatography.

In occurrences of the instances of the eighth aspect, the isolated and/or purified at least one cGAS type enzyme may be activated via addition of deoxyribonucleic acid at a ratio between 0.1 to 1 gram of deoxyribonucleic acid to 1 gram of cGAS type enzyme.

In a ninth aspect of the first embodiment, the reacting is conducted in the presence of at least one Solvent A.

In a first instance of the ninth aspect, the at least one Solvent A is selected from the group consisting of organic solvents, organic solvents in combination with water, and mixtures thereof. In occurrences of this instance, the at least one Solvent A is selected from the group consisting of organic solvents in combination with water. In instances of this aspect, the at least one Solvent A is selected from the group consisting of tetraglyme dimethyl ether (TGDE), MeCN, MeOH, EtOH, DMSO, propyl nitrile, sulfolane, pyrrolidone, 2-ethoxyl acetate, cyclohexanol, methyl pentyl ketone, cyclohexanone, 1,2,3,4-tetrahydronaphthalene, pivolate methyl ester, 2-methyl-3-butene-2-ol, tert-butanol, DMF, tetra-methyl urea, tetramethylene sulfone (also sulfolane or 1λ6-thiolane-1,1-dione), N,N-diethyl acetamide, ethylene glycol, NMP, isopropyl alcohol, 1-methoxy-2-propyl acetate (MPA), and mixtures thereof. In a specific instance of this aspect, the at least one Solvent A is TGDE. In specific instances of this aspect, the at least one Solvent A is provided in an amount in a range of from about 10 to 50 volumes with respect to the amount of the compound of Formula (I-1).

In a second instance of the ninth aspect, the at least one Solvent A is water. In specific occurrences of this instance, water is provided in an amount in a range of from about 10 to 1500 volumes with respect to the amount of the compound of Formula (I-1). In specific occurrences of this instance, the reaction is conducted in 50-200 volumes with respect to the amount of the compound of Formula (I-1).

In a tenth aspect of the first embodiment, the reacting is conducted in the presence of at least one Phosphatase Inhibitor A selected from the group consisting of Na₃VO₄, Na₂P₂O₇, (HOCH₂)₂CH—OP(O)(ONa), EDTA, Na₂WO₄, Na₂MO₄, NaF, KF, CsF, and mixtures thereof. In instances of this aspect, the at least one Phosphatase Inhibitor A is Na₃VO₄. In further instances, the at least one Phosphatase Inhibitor A is provided in an amount in a range of from about 0.005 to 2.0 equivalents with respect to the amount of the compound of Formula (I-1).

In an eleventh aspect of the first embodiment, the reacting is conducted in the presence of at least one Buffer A selected from the group consisting of 2-[[1,3-dihydroxy-2-(hydroxymethyl)propan-2-yl]amino]ethanesulfonic acid (TES), 2-amino-2-(hydroxymethyl) propane-1,3-diol (Tris), 2-[4-(2-hydroxyethyl)piperazin-1-yl]ethanesulfonic acid (HEPES), 3-[[1,3-dihydroxy-2-(hydroxymethyl)propan-2-yl]amino]-2-hydroxypropane-1-sulfonic acid (TAPSO), 2-morpholin-4-ylethanesulfonic acid (MES), and mixtures thereof. In a specific instance of this aspect, the at least one Buffer A is 2-[[1,3-dihydroxy-2-(hydroxymethyl)propan-2-yl]amino]ethanesulfonic acid (TES). In specific instances of this aspect, the at least one Buffer A is provided in an amount in a range of from about 0.1 to 30 equivalents with respect to the amount of the compound of Formula (I-1).

In a twelfth aspect of the first embodiment, the reacting is conducted in the presence of Base B, which is selected from the group consisting of KOH, NaOH, CsOH, (NH)₄OH, and mixtures thereof. In specific instances, Base B is KOH. In instances of this embodiment, Base B is included in an amount sufficient to control pH in a range of from about 7.1 to about 7.7. In specific occurrences of this instance, Base B is KOH, and pH is about 7.4.

In a thirteenth aspect of the first embodiment, the reacting is conducted in a temperature range of from about 5° C. to about 50° C. In instances of this aspect, the reacting is conducted in a temperature range of from about 25° C. to about 40° C.

In a fourteenth aspect of the first embodiment, the process further comprises forming a salt of the compound of Formula (I), which is a salt of the compound of Formula (Ia). In instances of this aspect, the compound of Formula (I) is a sodium, potassium, magnesium, cobalt, zinc, or ammonium salt. In specific instances, the compound of Formula (I) is a sodium or potassium salt. In instances of this aspect, the compound of Formula (I) comprises a cation selected from the group consisting of Na⁺, K⁺, Mg⁺⁺, Co⁺⁺, Zn⁺⁺, and NH₄⁺. In instances of this aspect, the compound of Formula (I) comprises two cations independently selected from the group consisting of Na⁺, K⁺, and NH₄⁺. In more specific instances, the compound of Formula (I) is a disodium salt or a dipotassium salt. In even more specific instances, the compound of Formula (I) is a disodium salt.

In a second embodiment, the process of the first embodiment further comprises preparing the compound of Formula (I-1) by reacting a compound of Formula (I-1a) with at least one guanylate kinase type enzyme and at least one acetate kinase enzyme.

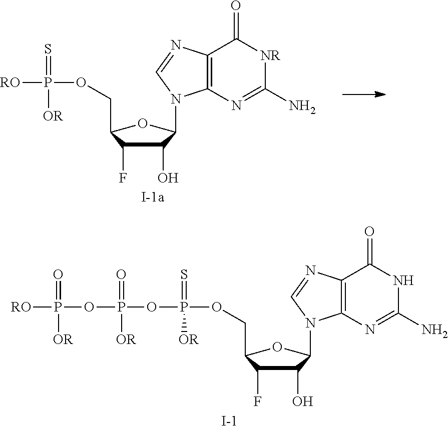

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In a first aspect of the second embodiment, the at least one guanylate kinase type enzyme is selected independently from the group consisting of wild-type guanylate kinase type enzymes and guanylate kinase enzymes that are the product of directed evolution from a wild-type guanylate kinase type enzyme. In specific instances, the at least one guanylate kinase type enzyme is the wild-type guanylate kinase type enzyme having the amino acid sequence that is SEQ ID NO: 14.

(SEQ ID NO: 14)
MALPRPVVICGPSGSGKSTLYNKLLKEFPGVFQLSVSHTTRQPRPGELNG
REYHFINRDQFQENIKQGDFLEWAEFSGNIYGTSKKALEEVQSNNVIPIL
DIDTQGVRNVKKASLEAVYIFIKPPSIDVLEKRLRSRKTETEEALQKRLS
AARNELEYGLKPGNFQHIITNDDLDVAYEKLKGILIKSQMPLAMATGSSS
SVVNSFLDKPAASATTVNSSSQD.

In a first instance of this first aspect, the at least one guanylate kinase type enzyme has the amino acid sequence that is SEQ ID NO: 15.

(SEQ ID NO: 15)
MHHHHHHALPRPVVICGPSGSGKSTLYNKLLKEFPGVFQLSVSHTTRQPR
PGELNGREYHFINRDQFQENIKQGDFLEWAEFSGNLYGTSKKALEEVQSN
NVIPILDIDTQGVRNVKKASLEAVYIFIKPPSIDVLEKRLRSRKTETEEA
LQKRLSAARNELEYGLKPGNFQHIITNDDLDVAYEKLKGILIKSQMPLAM
ATGSSSSVVNSFLDKPAASATTVNSSSQD.

In a second instance of this first aspect, the at least one guanylate kinase type enzyme has the amino acid sequence that is SEQ ID NO: 16.

(SEQ ID NO: 16)
MHHHHHHALPTPVVICGPSGSGKTTLYNKLLKEFPGVFQLSVSHTTRQPR
PGEENGREFHFINRDQFQENIKQGDFLEWAEFSGNLYGTSKKALEEVQAN
NVIPILDIDTQGVRNVKKASLEAVYIFIKPPSIDVLEERLRSRKTETEEA
LQKRLSAARNELEYGLKPGNFQHIITNDDLDVAYEKLKGILIKSQMPLAM
ATGSSSSVVNSFLDKPAASATTVNSSSQD

In a third instance of this first aspect, the at least one guanylate kinase type enzyme has the amino acid sequence that is SEQ ID NO: 17.

(SEQ ID NO: 17)
MHHHHHHALPTPVVICGPSGSGKTTLYNKLLKEFPGVFQLSVSHTTRQPR
PGEENGREFHFINRDQFQENIKQGDFLEWAEFSGNLYGTSKKALEEVQAN
NVIPILDIDTQGVRNVKKASLEAVYIFIKPPSIDVLEERLRSRKTETEEA
LQKRLSAAPNELEYGLKPGNFQHIITNDDLDVAYEKLKGILIKSQMPLAM
ATGSSSSVVNSFLDKPAASATTVNSSSQD

In a fourth instance of this first aspect, the at least one guanylate kinase type enzyme has the amino acid sequence that is SEQ ID NO: 18.

(SEQ ID NO: 18)
MHHHHHHALPTPVVICGPSGSGKTTLYNKLLKEFPGVFQLSVSHTTRQPR
PGEENGREFHFINRDQFQENIKQGDFLEWAEHSGNLYGTSKKALEEVQAN
NVIPILDIDTQGVRTVKKASLEAVYIFIKPPSIDVLEERLRSRGTETEEA
LQKRLSAAPNELEYGLKPGNFQHIITNDDLDVAYEKLKGILIDSQMPLAG
ATGSSSSVVNSFLDKPAASATTVNSSSQD

In a fifth instance of this first aspect, the at least one guanylate kinase type enzyme has the amino acid sequence that is SEQ ID NO: 19.

(SEQ ID NO: 19)
MHHHHHHALPTPVVICGPSGSGKTTLYNKLLKEFPGVFQLTASHTTRQPR
PGEENGREFHFINRDQFQENIKQGDFLEWAEHSGNLYGTSKKALEEVQAN
NVIPILDIDTQGVRTVKKASLEAVYIFIKPPSIEVLEERLRSRGTETEEA
LQKRLSAAPNELEYGLKPGNFQHIITNDDLDVAYEKLKGILIDSQMPLAG
ATGSSSSVVNSFLDKPAASATTVNSSSQD

In even more specific instances of this aspect, the at least one guanylate kinase type enzyme is selected from wild-type guanylate kinase type enzyme, which has the amino acid sequence that is SEQ ID NO: 14, and guanylate kinase type enzymes that are the product of directed evolution from a wild-type guanylate kinase type enzyme and that have the amino acid sequences that is SEQ ID NO: 15, SEQ ID NO: 16, SEQ ID NO: 17, SEQ ID NO: 18, and SEQ ID NO: 19.

In a sixth instance of this first aspect, the at least one guanylate kinase type enzyme is selected from the group consisting of guanylate kinase type enzymes and immobilized guanylate kinase type enzymes. In particular instances, each of the at least one guanylate kinase type enzymes is independently selected from the group consisting of guanylate kinase type enzymes and immobilized guanylate kinase type enzymes, such that there may be mixtures of enzymes in which all, some, or no enzymes are immobilized. In particular instances, the at least one guanylate kinase type enzyme comprises one or more immobilized kinase type enzymes that are immobilized independently, on separate resins. In other particular instances, the at least one guanylate kinase type enzyme comprises one or more immobilized guanylate kinase type enzymes that are co-immobilized on a single resin.

In a first occurrence of this sixth instance, the at least one guanylate kinase type enzyme is immobilized on at least one hydrophilic resin.

In a first facet of this first occurrence, the at least one guanylate kinase type enzyme is immobilized by covalent bonds on the at least on hydrophilic resin that includes at least one exposed ligand that can be further reacted. In specific examples of this first facet, the at least one exposed ligand comprises a functional group ligand or functional group selected from the group consisting of aryl, biotin, desthiobiotin, thiol, amine, amide, alkoxy, acetal, ketal, ester, anhydride, carbonyl, nitrile, epoxy, carboxyamide, ammonium, iodo, phenolic, imidazolyl, morpholinyl, pyridyl, phenyl, sulfide, disulfide, sulfhydryl ketone, acyl chloride, imine, nitrile, anilino, nitro, halo, alkyl, hydroxyl, maleimide, iodoacetyl, triazine, sulfonate, alkylamine, diol, hydrazide, hydrazine, azlactone, aldehyde, diazonium, carboxylate, azide, vinyl sulfone, epoxide, and oxirane groups, and combinations thereof. In more specific examples, the al least one ligand may be further reacted with a homobifunctional or heterobifunctional spacer arm to impart identical or different functionality. Spacer arms are well known in the art and include but not limited to (C₂-C₂₀)alkylene groups that may incorporate one or more hetero atom, aromatic groups, alkylaromatic groups, amido groups, amino groups, urea groups, carbamate groups, ether groups, thio ether groups, and the like, and combinations thereof. In even more specific instances, the spacer arm is one or more selected from the group consisting of ethylenediamine, 1,3-diamino-2-propanol, diaminodipropylamine (DADPA), cystamine, 1,6-diaminohexane, O-(2-Aminopropyl)-O′-(2-nmethoxyethyl)polypropylene glycols such as Jeffanine™ ED-600, unhindered diamines such as Jeffanine™ FDR-148 polyetheranine, 4,7,10-trioxa-1,13-tridecanediamine, Boc-N-amido-dPEG₁₁-amine, Boc-N-amido-dPE3anine), beta-alanine, aminocaproic acid, amino-PEGₙ-carboxylate compounds (where n is between 2 and 20), succinic acid, succinic anhydride, glutaric acid, glutaric anhydride, diglycolic acid, diglycolic anhydride, thioglycolic acid, N-succinimidyl S-acetylthioacetate, N-succinimidyl S-acetylthiopropionate, N-acetyl homocysteine thiolactone, 8-mercaptooctanoic acid, alpha-lipoic acid, lipoamide-PEGₙ-carboxylate compounds, thiol-PEGₙ-carboxylate compounds, NHS-PEGₙ-acetylated thiol compounds, dithiothreitol (DTT), tetra(ethylene glycol) dithiol, hexa(ethylene glycol) dithiol, poly(ethylene glycol) dithiol, 2-mercaptoethylamine., adipic dihydrazide, and carbohydrazide.

In a second facet of this first occurrence, the at least one guanylate kinase type enzyme is immobilized on the at least one hydrophilic resin by non-covalent bonds. In specific facets, the at least one guanylate kinase type enzyme is immobilized by non-covalent bonds on a hydrophilic resin that includes at least one functional group selected from the group consisting of strong ion exchangers, weak ion exchangers, hydroxyapatite, multimodal ligands, hydrophilic modifiers, and hydrophobic modifiers, and mixtures thereof. In more specific examples, the at least one ligand is selected from the group consisting of quaternary ammonium, sulfopropyl, methyl sulfonate, diethylaminoethyl, and carboxymethyl.

In a second occurrence of this sixth instance, the at least one guanylate kinase type enzyme is immobilized on the at least one hydrophilic resin comprising at least one chelating ligand selected from the group consisting of iminodiacetic acid (IDA), nitrilotriacetic acid (NTA), tris carboxymethyl ethylene diamine (TED), and mixtures thereof. In facets of this occurrence, the at least one chelating ligand is NTA. In facets of this second occurrence, the at least one hydrophilic resin comprising at least one chelating ligand comprises at least one metal ion selected from the group consisting of Fe²⁺, Cu²⁺, Mg²⁺, Zn²⁺, Co²⁺, and Ni²⁺. In specific examples, the at least one metal ion is Ni²⁺.

In a third occurrence of this sixth instance, the at least one guanylate kinase type enzyme is immobilized on the at least one hydrophilic resin by use of at least one Buffer B is selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl)piperazin-1-yl]ethanesulfonic acid (HEPES), sodium acetate, potassium acetate, and mixtures thereof. In a specific facet, the at least one Buffer B is selected from the group consisting of sodium phosphate solutions, potassium phosphate solutions, and mixtures thereof. In specific facets, the at least one Buffer B is provided in an amount sufficient to adjust the pH to a range of from about 6 to about 10, such as an amount sufficient to adjust the pH to a range of from about 6.8 to about 8.2.

In facets of the third occurrence of this sixth instance, the at least one guanylate kinase type enzyme is immobilized on the at least one hydrophilic resin by dissolving a lyophilized powder of the at least one guanylate kinase type enzyme in at least one Buffer B, at a concentration of from about 1 grams per liter (g/l) to about 100 g/l. In particular examples of these facets, the at least one guanylate kinase type enzyme at a concentration of from about 5 g/l to about 25 g/l. In additional examples of these facets, the at least one guanylate kinase type enzyme is 2 or more enzymes which are dissolved in a single vessel, in a gravimetric ratio of from about 1:1 to about 1:100, such as from about 1:1 to about 1:10. In specific examples of these facets, the total concentration of the one or more guanylate kinase type enzyme is from about 1 g/l to about 100 g/l, such as from about 5 g/l to about 25 g/l.

In a fourth occurrence of this sixth instance, the at least one guanylate kinase type enzyme is immobilized on the at least one hydrophilic resin in the presence of imidazole, NaCl, or mixtures thereof. In facets of this occurrence, the imidazole is present in an amount of from about 0 mM to about 30 mM, and the NaCl is present in an amount of about 500 mM.

In a fifth occurrence of this sixth instance, the at least one guanylate kinase type enzyme is immobilized on the at least one hydrophilic resin by incubating the one or more guanylate kinase type enzyme and the at least one hydrophilic resin in an agitated vessel at a temperature of from about 2° C. to about 30° C. over a period of from 10 minutes to 1 week. In specific facets of this occurrence, incubating is in an agitated vessel at a temperature of from about 2° C. to about 6° C. over a period of from 2 hours and 2 days. In other specific facets of this occurrence, incubating is in an agitated vessel at a temperature of from about 20° C. to about 25° C. over a period of from 30 minutes and 1 day.

In a sixth occurrence of this sixth instance, the at least one guanylate kinase type enzyme is immobilized on the at least one hydrophilic resin by passing a solution containing the at least one guanylate kinase type enzyme through a packed bed reactor containing the at least one hydrophilic resin dissolved in a Buffer C. In facets of this occurrence, the Buffer C is selected from the group consisting of sodium phosphate solutions, potassium phosphate solutions, and mixtures thereof. In specific facets of this occurrence, the at least one Buffer C is provided in an amount sufficient to adjust the pH to a range of from about 6 to about 10, such as an amount sufficient to adjust the pH to a range of from about 6.8 to about 8.2. In additional facets of this occurrence, the at least one Buffer C comprises imidazole, NaCl, or mixtures thereof.

In a seventh occurrence of this sixth instance, the at least one hydrophilic resin is clarified by washing with a Buffer D having a pH in a range of from about 6.8 to about 8.5 and comprising one or more reagent selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl) piperazin-1-yl]ethanesulfonic acid (HEPES), sodium acetate, potassium acetate, NaCl, and imidazole, and mixtures thereof. In facets of this seventh occurrence, the Buffer D comprises NaCl, imidazole, or mixtures thereof, such as mixtures of from about OM to about 1M NaCl and of from about 0 mM to about 300 mM imidazole. In further facets of this seventh occurrence, the Buffer D comprises sodium phosphate, NaCl, imidazole, or mixtures thereof, such as mixtures of from about 50 mM to about 100 mM sodium phosphate, of from about 300 mM to about 500 mM NaCl and of from about 0 mM to about 50 mM imidazole. In still further facets of this seventh occurrence, the Buffer D comprises potassium phosphate, NaCl, imidazole, or mixtures thereof, such as mixtures of from about 50 mM to about 100 mM potassium phosphate, of from about 300 mM to about 500 mM NaCl and of from about 0 mM to about 50 mM imidazole. In facets of this eighth occurrence, the Buffer D has a concentration of from about 0 mM to about 100 mM.

In a second aspect of the second embodiment, the at least one acetate kinase type enzyme is selected independently from the group consisting of wild-type acetate kinase type enzymes and acetate kinase enzymes that are the product of directed evolution from a wild-type acetate kinase type enzyme. In specific instances, the at least one acetate kinase type enzyme is the wild-type acetate kinase type enzyme having the amino acid sequence that is SEQ ID NO: 20.

(SEQ ID NO: 20)
MRVLVINSGSSSIKYQLIEMEGEKVLCKGIAERIGIEGSRLVHRVGDEKH
VIERELPDHEEALKLILNTLVDEKLGVIKDLKEIDAVGHRVVHGGERFKE
SVLVDEEVLKAIEEVSPLAPLHNPANLMGIKAAMKLLPGVPNVAVFDTAF
HQTIPQKAYLYAIPYEYYEKYKIRRYGFHGTSHRYVSKRAAEILGKKLEE
LKIITCHIGNGASVAAVKYGKCVDTSMGFTPLEGLVMGTRSGDLDPAIPF
FIMEKEGISPQEMYDILNKKSGVYGLSKGFSSDMRDIEEAALKGDEWCKL
VLEIYDYRIAKYIGAYAAAMNGVDAIVFTAGVGENSPITREDVCSYLEFL
GVKLDKQKNEETIRGKEGIISTPDSRVKVLVVPTNEELMIARDTKEIVEK
IGR.

In a first instance of this second aspect, the at least one acetate kinase type enzyme has the amino acid sequence that is SEQ ID NO: 21.

(SEQ ID NO: 21)
MGSHHHHHGSRVLVINSGSSSIKYQLIEMEGEKVLCKGIAERIGIEGSRL
VHRVGDEKHVIERELPDHEEALKLILNTLVDEKLGVIKDLKEIDAVGHRV
VHGGERFKESVLVDEEVLKAIEEVSPLAPLHNPANLMGIKVAMKLLPGVP
NVAVFDTAFHQTIPQKAYLYAIPYEYYEKYKIRRYGFHGTSHRYVSKRAA
EILGKKLEELKIITCHIGNGASVAAVKYGKCVDTSMGFTPTEGLVMGTRS
GDLDPAIPFFIMEKEGISPQEMYDILNKKSGVYGLSKGFSSDMRDIEEAA
LKGDEWCKLVLEIYDYRIAKYIGAYAAAMNGVDAIVFTAGVGENSPITRE
DVCSYLEFLGVKLDKQKNEETIRGKEGIISTPDSRVKVLVVPTNEELMIA
RDTKEIVEKIGR.

In a second instance of this second aspect, the at least one acetate kinase type enzyme has the amino acid sequence that is SEQ ID NO: 22.

(SEQ ID NO: 22)
MGSHHHHHGSRVLVINSGSSSIKYQLIEMEGEKVLCKGIAERIGIEGSRL
VHRVGDEKHVIERELPDHEEALKLILNTLVDEKLGVIKDLKEIDAVGHRV
VHGGERFKESVLVDEEVLKAIEEVSPLAPLHNPANLMGIKVAMKLLPGVP
NVAVFDTAFHQTIPQKAYLYAIPYEYYEKYKIRRYGFHGTSHRYVSKRAA
EILGKKLEELKIITCHIGNGASVAAVKYGKCVDTSMGFTPTEGLVMGTRS
GDLDPAIPFFIMEKEGISPQEMYDILNKKSGVYGLSKGFSSDLRDIEEAA
LKGDEWCKLVLEIYDYRIAKYIGAYAAAMNGVDAIVFTAGVCENSPITRE
DVCSYLEFLGVKLDKQKNEETIDGKEGIISTPDSRVKVLVVPTNEELMIA
RDTKEIVEKIGR

In even more specific instances of this aspect, the at least one acetate kinase type enzyme is selected from wild-type acetate kinase type enzyme, which has the amino acid sequence that is SEQ ID NO: 20, and acetate kinase type enzymes that are the product of directed evolution from a wild-type acetate kinase type enzyme and that have the amino acid sequence that is SEQ ID NO: 21 and SEQ ID NO: 22.

In a third instance of this second aspect, the at least one acetate kinase type enzyme is selected from the group consisting of acetate kinase type enzymes and immobilized acetate kinase type enzymes. In particular instances, each of the at least one acetate kinase type enzyme is independently selected from the group consisting of acetate kinase type enzymes and immobilized acetate kinase type enzymes, such that there may be mixtures of enzymes in which all, some, or no enzymes are immobilized. In particular instances, the at least one acetate kinase type enzyme comprises one or more immobilized kinase type enzymes that are immobilized independently, on separate resins. In other particular instances, the at least one acetate kinase type enzyme comprises one or more immobilized acetate kinase type enzymes that are co-immobilized on a single resin.

In a first occurrence of this third instance, the at least one acetate kinase type enzyme is immobilized on at least one hydrophilic resin.

In a first facet of this first occurrence, the at least one acetate kinase type enzyme is immobilized by covalent bonds on the at least on hydrophilic resin that includes at least one exposed ligand that can be further reacted. In specific examples of this first facet, the at least one exposed ligand comprises a functional group ligand or functional group selected from the group consisting of aryl, biotin, desthiobiotin, thiol, amine, amide, alkoxy, acetal, ketal, ester, anhydride, carbonyl, nitrile, epoxy, carboxyamide, ammonium, iodo, phenolic, imidazolyl, morpholinyl, pyridyl, phenyl, sulfide, disulfide, sulfhydryl ketone, acyl chloride, imine, nitrile, anilino, nitro, halo, alkyl, hydroxyl, maleimide, iodoacetyl, triazine, sulfonate, alkylamine, diol, hydrazide, hydrazine, azlactone, aldehyde, diazonium, carboxylate, azide, vinyl sulfone, epoxide, and oxirane groups, and combinations thereof. In more specific examples, the at least one ligand may be further reacted with a homobifunctional or heterobifunctional spacer arm to impart identical or different functionality. Spacer arms are w ell known in the art and include but not limited to (C₂-C₂₀)alkylene groups that may incorporate one or more hetero atom, aromatic groups, alkylaromatic groups, amido groups, amino groups, urea groups, carbamate groups, ether groups, thio ether groups, and the like, and combinations thereof. In even more specific instances, the spacer arm is one or more selected from the group consisting of ethylenediamine, 1,3-diamino-2-propano, diaminodipropylamine (DADPA), cystamine, 1,6-diaminohexane, O-(2-Aminopropyl)-O′-(2-methoxyethyl)polypropylene glycols such as Jeffamine, ED-600 unhindered diamines such as Jeffamine™ EDR-148 polyetheramine, 4,7,10-troxa-1,13-tridecanediamine, Boc-N-amido-dPEG₁₁-amine, Boc-N-amido-dPEG₃-amine), beta-alanine, aminocaproic acid, amino-PEGₙ-carboxylate compounds (where n is between 2 and 20), succinic acid, succinic anhydride, glutaric acid, glutaric anhydride, diglycolic acid, diglycolic anhydride, thioglycolic acid, N-succinimidyl S-acetylthioacetate, N-succinimidyl S-acetylthiopropionate, N-acetyl homocysteine thiolactone, 8-mercaptooctanoic acid, alpha-lipoic acid, lipoamide-PEGₙ-carboxylate compounds, thiol-PEG-carboxylate compounds, NHS-PEGₙ-acetylated thiol compounds, dithiothreitol (DTT)., tetra(ethylene gly col) dithiol, hexa(ethylene glycol) dithiol, poly(ethylene glycol) dithiol, 2-mercaptoethylamine, adipic dihydrazide, and carbohydrazide.

In a second facet of this first occurrence, the at least one acetate kinase type enzyme is immobilized on the at least one hydrophilic resin by non-covalent bonds. In specific facets, the at least one acetate kinase type enzyme is immobilized by non-covalent bonds on a hydrophilic resin that includes at least one functional group selected from the group consisting of strong ion exchangers, weak ion exchangers, hydroxyapatite, multimodal ligands, hydrophilic modifiers, and hydrophobic modifiers, and mixtures thereof. In more specific examples, the at least one ligand is selected from the group consisting of quaternary ammonium, sulfopropyl, methyl sulfonate, diethylaminoethyl, and carboxymethyl.

In a second occurrence of this third instance, the at least one acetate kinase type enzyme is immobilized on the at least one hydrophilic resin comprising at least one chelating ligand selected from the group consisting of iminodiacetic acid (IDA), nitrilotriacetic acid (NTA), tris carboxymethyl ethylene diamine (TED), and mixtures thereof. In facets of this occurrence, the at least one chelating ligand is NTA. In facets of this second occurrence, the at least one hydrophilic resin comprising at least one chelating ligand comprises at least one metal ion selected from the group consisting of Fe²⁺, Cu²⁺, Mg²⁺, Zn²⁺, Co²⁺, and Ni²⁺. In specific examples, the at least one metal ion is Ni²⁺.

In a third occurrence of this third instance, the at least one acetate kinase type enzyme is immobilized on the at least one hydrophilic resin by use of at least one Buffer E selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl)piperazin-1-yl]ethanesulfonic acid (HEPES), sodium acetate, potassium acetate, and mixtures thereof. In a specific facet, the at least one Buffer E is selected from the group consisting of sodium phosphate solutions, potassium phosphate solutions, and mixtures thereof. In specific facets, the at least one Buffer E is provided in an amount sufficient to adjust the pH to a range of from about 6 to about 10, such as an amount sufficient to adjust the pH to a range of from about 6.8 to about 8.2.

In facets of the third occurrence of this third instance, the at least one acetate kinase type enzyme is immobilized on the at least one hydrophilic resin by dissolving a lyophilized powder of the at least one acetate kinase type enzyme in at least one Buffer E, at a concentration of from about 1 g/l to about 100 g/l. In particular examples of these facets, the at least one acetate kinase type enzyme at a concentration of from about 5 g/l to about 25 g/l. In additional examples of these facets, the at least one acetate kinase type enzyme is 2 or more enzymes which are dissolved in a single vessel, in a gravimetric ratio of from about 1:1 to about 1:100, such as from about 1:1 to about 1:10. In specific examples of these facets, the total concentration of the one or more acetate kinase type enzyme is from about 1 g/l to about 100 g/l, such as from about 5 g/l to about 25 g/l.

In a fourth occurrence of this third instance, the at least one acetate kinase type enzyme is immobilized on the at least one hydrophilic resin in the presence of imidazole, NaCl, or mixtures thereof. In facets of this occurrence, the imidazole is present in an amount of from about 0 mM to about 30 mM, and the NaCl is present in an amount of about 500 mM.

In a fifth occurrence of this third instance, the at least one acetate kinase type enzyme is immobilized on the at least one hydrophilic resin by incubating the one or more acetate kinase type enzyme and the at least one hydrophilic resin in an agitated vessel at a temperature of from about 2° C. to about 30° C. over a period of from 10 minutes to 1 week. In specific facets of this occurrence, incubating is in an agitated vessel at a temperature of from about 2° C. to about 6° C. over a period of from 2 hours and 2 days. In other specific facets of this occurrence, incubating is in an agitated vessel at a temperature of from about 20° C. to about 25° C. over a period of from 30 minutes and 1 day.

In a sixth occurrence of this third instance, the at least one acetate kinase type enzyme is immobilized on the at least one hydrophilic resin by passing a solution containing the at least one acetate kinase type enzyme through a packed bed reactor containing the at least one hydrophilic resin dissolved in a Buffer G. In facets of this occurrence, the Buffer G is selected from the group consisting of sodium phosphate solutions, potassium phosphate solutions, and mixtures thereof. In specific facets of this occurrence, the at least one Buffer G is provided in an amount sufficient to adjust the pH to a range of from about 6 to about 10, such as an amount sufficient to adjust the pH to a range of from about 6.8 to about 8.2. In additional facets of this occurrence, the at least one Buffer G comprises imidazole, NaCl, or mixtures thereof.

In a seventh occurrence of this third instance, the at least one hydrophilic resin is clarified by washing with a Buffer H having a pH in a range of from about 6.8 to about 8.5 and comprising one or more reagent selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl) piperazin-1-yl]ethanesulfonic acid (HEPES), sodium acetate, potassium acetate, NaCl, and imidazole, and mixtures thereof. In facets of this seventh occurrence, the Buffer H comprises NaCl, imidazole, or mixtures thereof, such as mixtures of from about OM to about 1M NaCl and of from about 0 mM to about 300 mM imidazole. In further facets of this seventh occurrence, the Buffer H comprises sodium phosphate, NaCl, imidazole, or mixtures thereof, such as mixtures of from about 50 mM to about 100 mM sodium phosphate, of from about 300 mM to about 500 mM NaCl and of from about 0 mM to about 50 mM imidazole. In still further facets of this seventh occurrence, the Buffer H comprises potassium phosphate, NaCl, imidazole, or mixtures thereof, such as mixtures of from about 50 mM to about 100 mM potassium phosphate, of from about 300 mM to about 500 mM NaCl and of from about 0 mM to about 50 mM imidazole. In facets of this eighth occurrence, the Buffer H has a concentration of from about 0 mM to about 100 mM.

In additional aspects, the at least one guanylate kinase type enzyme and the at least one acetate kinase type enzyme may be co-immobilized.

In a third aspect of the second embodiment, the reacting is conducted in the presence of at least one Co-Factor A. In instances of this aspect, the at least one Co-Factor A is 2′F-thio-ATP or natural ATP. In specific instances of this aspect, the at least one Co-Factor A is provided in an amount in a range of from about 0.0005 to 2.0 equivalents with respect to the amount of the compound of Formula (I-1a).

In a fourth aspect of the second embodiment, the reacting is conducted in the presence of at least one Metal Co-Factor B. In instances of this aspect, the at least one Metal Co-Factor B is selected from the group consisting of MgCl₂, MnCl₂, and Mg(OH)₂, hydrates thereof, and mixtures thereof. In specific instances, the at least one Metal Co-Factor B is MgCl₂. In specific instances of this aspect, the at least one Metal Co-Factor B is provided in an amount in a range of from about 0.1 to 5.0 equivalents with respect to the amount of the compound of Formula (I-1a).

In a fifth aspect of the second embodiment, the reacting is conducted in the presence of at least one Inorganic Salt D selected from the group consisting of KCl, KBr, and NaCl, and mixtures thereof. In specific instances of this aspect, the at least one Inorganic Salt D is KCl. In specific instances of this aspect, the at least one Inorganic Salt D is provided in an amount in a range of from about 0.1 to 10.0 equivalents with respect to the amount of the compound of Formula (I-1a).

In a sixth aspect of the second embodiment, the reacting is conducted in the presence of at least one Salt A selected from the group consisting of AcP—Li/Li, AcP—Na/Na, AcP—K/K, AcP—Li/K, AcP—NH₄/NH₄, and mixtures thereof. In specific instances of this aspect, the at least one Salt A is AcP—Li/Li. In specific instances of this aspect, the at least one Salt A is provided in an amount in a range of from about 0.5 to 7.0 equivalents with respect to the amount of the compound of Formula (I-1a).

In a seventh aspect of the second embodiment, the reacting is conducted in the presence of at least one Solvent B.

In a first instance of the seventh aspect, the at least one Solvent B is water. In specific occurrences of this instance, water is provided in an amount in a range of from about 5 to 15 volumes with respect to the amount of the compound of Formula (I-1a).

In a second instance of the seventh aspect, the at least one Solvent B is selected from water in combination with at least one organic solvent. In instances of this aspect, the at least one Solvent B is selected from the group consisting of EtOH, MeOH, iPrOH, MeCN, DMSO, TGDE, EtOAc, acetone, and tBuOH, and mixtures thereof. In a specific instance of this aspect, the at least one Solvent B is water in combination with EtOH.

In an eighth aspect of the second embodiment, the reacting is conducted in a temperature range of from about −10° C. to about 35° C. In instances of this aspect, the reacting is conducted in a temperature range of from about 0° C. to about 25° C.

In a ninth aspect of the second embodiment, the reacting is conducted in the presence of Base C, which is selected from the group consisting of KOH, NaOH, and mixtures thereof. In specific instances, Base C is KOH. In other specific instances, Base C is NaOH. In instances of this embodiment, Base C is included in an amount sufficient to control pH in a range of from about 5.5 to about 8.5. In instances of this embodiment, Base C is included in an amount sufficient to control pH in a range of from about 6.4 to about 7.0 at a temperature of about 25° C.

In a third embodiment, the process of the first embodiment further comprises preparing the compound of Formula (I-2) by reacting a compound of Formula (I-2a) with at least one acetate kinase type enzyme and at least one adenylate kinase enzyme.

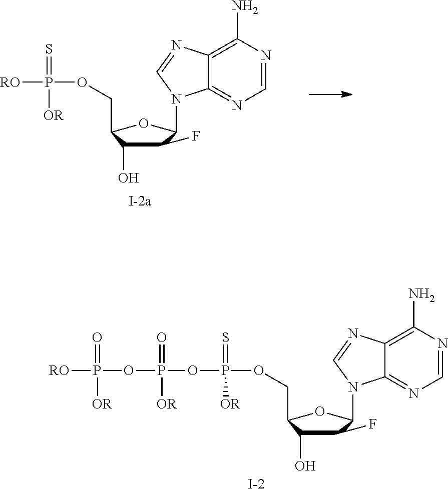

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In a first aspect of the third embodiment, the at least one acetate kinase type enzyme is as described above with respect to the second embodiment. That is, the at least one acetate kinase type enzyme is selected independently from the group consisting of wild-type acetate kinase type enzymes and acetate kinase enzymes that are the product of directed evolution from a wild-type acetate kinase type enzyme. In specific instances, the at least one acetate kinase type enzyme is the wild-type acetate kinase type enzyme having the amino acid sequence that is SEQ ID NO: 20. In a first instance of this first aspect, the at least one acetate kinase type enzyme has the amino acid sequence that is SEQ ID NO: 21. In a second instance of this first aspect, the at least one acetate kinase type enzyme has the amino acid sequence that is SEQ ID NO: 22.

In a third instance of this first aspect, the at least one acetate kinase type enzyme is selected from the group consisting of acetate kinase type enzymes and immobilized acetate kinase type enzymes, as described above.

In a second aspect of the third embodiment, the at least one adenylate kinase enzyme is selected independently from the group consisting of wild-type adenylate kinase type enzymes and adenylate kinase enzymes that are the product of directed evolution from a wild-type adenylate kinase type enzyme. In specific instances, the at least one adenylate kinase type enzyme is the wild-type adenylate kinase type enzyme having the amino acid sequence that is SEQ ID NO: 23.

(SEQ ID NO: 23)
MSSSESIRMVLIGPPGAGKGTQAPNLQERFHAAHLATGDMLRSQIAKGTQ
LGLEAKKIMDQGGLVSDDIMVNMIKDELTNNPACKNGFILDGFPRTIPQA
EKLDQMLKEQGTPLEKAIELKVDDELLVARITGRLIHPASGRSYHKIFNP
PKEDMKDDVTGEALVQRSDDNADALKKRLAAYHAQTEPIVDFYKKTGIWA
GVDASQPPATVWADILNKLGKD.

In a first instance of this second aspect, the at least one adenylate kinase type enzyme has the amino acid sequence that is SEQ ID NO: 24.

(SEQ ID NO: 24)
MHHHHHHSSSESIRMVLIGPPGAGKGTQAPNLQERFHACHLATGDMLRSQ
IAKGTQLGLEAKKIMDQGGLVSDDIMVNMIKDELTNNPACKNGFILDGFP
RTIPQAEKLDQMLKEQGTPLEKAVELKVDDELLVARITGRLIHPASGRSY
HKIFNPPKEDMKDDVTGEALVQRSDDNADALKKRLAAYHAQTEPVVDFYK
KTGIWAGVDASQPPATVWADILNKLGKD.

In a second instance of this second aspect, the at least one adenylate kinase type enzyme has the amino acid sequence that is SEQ ID NO: 25.

(SEQ ID NO: 25)
MHHHHHHSSSESIRMVLIGPPGAGKGTQAPNLQERFHACHLATGDMLRSQ
IAKGTQLGLEAKKIMDQGGLVSDDIMVNMIKDELTNNPACKNGFILDGFP
RTIPQAEKLDQMLKEQGTPLEKAVELKIDDELLPARITGRLIHPASGRSY
HKIFNPPKEDMKDDVTGEALVQRSDDNADALKKRLAAYHKQTEPVVDFYK
KTGIWAGVDASQPPATVWADILNKLGKD.

In a third instance of this second aspect, the at least one adenylate kinase type enzyme has the amino acid sequence that is SEQ ID NO: 26.

(SEQ ID NO: 26)
MHHHHHHSSSESIRMVLIGPPGAGKGTQAPNLQERFHACHLGTGDMLRSQ
IAKGTQLGLEAKKIMDQGGLVSDDIMVNMIKDELTNNPACKNGFILDGFP
RTIPQAEKLDQMLKEQGTPLEKAVELKIDDELLPARITGRLIHPASGRSY
HKIFNPPKEDMKDDVTGEALVQRSDDNADALKKRLAAYHKQTEPVVDFYK
KTGIWAGVDASQPPATVWADILNKLGKD.

In a fourth instance of this second aspect, the at least one adenylate kinase type enzyme has the amino acid sequence that is SEQ ID NO: 27.

(SEQ ID NO: 27)
MHHHHHHSSSESIRMVLIGPPGAGKGTQSPNLQERFHACHLGTGDMLRSQ
HAKGTQLGLEAKKIMDQGGLVSDDIMVNMIKDELTNNPACKNGFILDGFP
RTIPQAMKLEQMLKEQGTPLEKAVELKIDDELLPARITGRLIHPASGRSY
HKIFNPPKEDMKDDVTGEALVQRSDDNADALKKRLAAYHKQTEPVVDFYK
KAGIWAGVDASQPVATVWADILNKLGKD

In even more specific instances of this aspect, the at least one adenylate kinase type enzyme is selected from wild-type adenylate kinase type enzyme, which has the amino acid sequence that is SEQ ID NO: 23, and adenylate kinase type enzymes that are the product of directed evolution from a wild-type adenylate kinase type enzyme and that have the amino acid sequence that is SEQ ID NO: 24, SEQ ID NO: 25, SEQ ID NO: 26, and SEQ ID NO: 27.

In a fifth instance of this second aspect, the at least one adenylate kinase type enzyme is selected from the group consisting of adenylate kinase type enzymes and immobilized adenylate kinase type enzymes. In particular instances, each of the at least one adenylate kinase type enzyme is independently selected from the group consisting of adenylate kinase type enzymes and immobilized adenylate kinase type enzymes, such that there may be mixtures of enzymes in which all, some, or no enzymes are immobilized. In particular instances, the at least one adenylate kinase type enzyme comprises one or more immobilized kinase type enzymes that are immobilized independently, on separate resins. In other particular instances, the at least one adenylate kinase type enzyme comprises one or more immobilized adenylate kinase type enzymes that are co-immobilized on a single resin.

In a first occurrence of this fifth instance, the at least one adenylate kinase type enzyme is immobilized on at least one hydrophilic resin.

In a first facet of this first occurrence, the at least one adenylate kinase type enzyme is immobilized by covalent bonds on the at least on hydrophilic resin that includes at least one exposed ligand that can be further reacted. In specific examples of this first facet, the at least one exposed ligand comprises a functional group ligand or functional group selected from the group consisting of aryl, biotin, desthiobiotin, thiol, amine, amide, alkoxy, acetal, ketal, ester, anhydride, carbonyl, nitrile, epoxy, carboxyamide, ammonium, iodo, phenolic, imidazolyl, morpholinyl, pyridyl, phenyl, sulfide, disulfide, sulfhydryl ketone, acyl chloride, imine, nitrile, anilino, nitro, halo, alkyl, hydroxyl, maleimide, iodoacetyl, triazine, sulfonate, alkylamine, diol, hydrazide, hydrazine, azlactone, aldehyde, diazonium, carboxylate, azide, vinyl sulfone, epoxide, and oxirane groups, and combinations thereof. In more specific examples, the at least one ligand may be further reacted with a homobifunctional or heterobifunctional spacer arm to impart identical or different functionality. Spacer arms are well known in the art and include but not limited to (C₂-C₂₀)alkylene groups that may incorporate one or more hetero atom, aromatic groups, alkylaromatic groups, amido groups, amino groups, urea groups, carbamate groups, ether groups, thio ether groups, and the like, and combinations thereof. In even more specific instances, the spacer arm is one or more selected from the group consisting of ethylenediamine, 1,3-diamino-2-propanol, diaminodipropylamine (DADPA), cystamine, 1,6-diaminohexane, O-(2-Aminopropyl)-O′-(2-methoxyethyl)polypropylene glycols such as Jeffamine™ ED-600, unhindered diamines such as Jeffanine™ EDR-148 polyetheranine, 4,7,10-trioxa-1,13-tridecanediamidine, Boc-N-amido-dPEG₁₁-amine, Boc-N-amido-dPEG₃-amine), beta-alanine, aminocaproic acid, amino-PEG-carboxylate compounds (where n is between 2 and 20), succinic acid, succinic anhydride, glutaric acid, glutaric anhydride, diglycolic acid, diglycolic anhydride, thioglycolic acid, N-succinimidyl S-acetylthioacetate, N-succinimidyl S-acetylthiopropionate, N-acetyl homocysteine thiolactone, 8-mercaptooctanoic acid, alpha-lipoic acid, lipoamide-PEGₙ-carboxylate compounds, thiol-PEGₙ-carboxylate compounds, NIS-PEG,-acetylated thiol compounds, dithiothreitol (DTI), tetra(ethylene glycol) dithiol, hexa(ethylene glycol) dithiol, poly(ethylene glycol) dithiol, 2-mercaptoethylamine, adipic dihydrazide, and carbohydrazide.

In a second facet of this first occurrence, the at least one adenylate kinase type enzyme is immobilized on the at least one hydrophilic resin by non-covalent bonds. In specific facets, the at least one adenylate kinase type enzyme is immobilized by non-covalent bonds on a hydrophilic resin that includes at least one functional group selected from the group consisting of strong ion exchangers, weak ion exchangers, hydroxyapatite, multimodal ligands, hydrophilic modifiers, and hydrophobic modifiers, and mixtures thereof. In more specific examples, the at least one ligand is selected from the group consisting of quaternary ammonium, sulfopropyl, methyl sulfonate, diethylaminoethyl, and carboxymethyl.

In a second occurrence of this fifth instance, the at least one adenylate kinase type enzyme is immobilized on the at least one hydrophilic resin comprising at least one chelating ligand selected from the group consisting of iminodiacetic acid (IDA), nitrilotriacetic acid (NTA), tris carboxymethyl ethylene diamine (TED), and mixtures thereof. In facets of this occurrence, the at least one chelating ligand is NTA. In facets of this second occurrence, the at least one hydrophilic resin comprising at least one chelating ligand comprises at least one metal ion selected from the group consisting of Fe²⁺, Cu²⁺, Mg²⁺, Zn²⁺, Co²⁺, and Ni²⁺. In specific examples, the at least one metal ion is Ni²⁺.

In a third occurrence of this fifth instance, the at least one adenylate kinase type enzyme is immobilized on the at least one hydrophilic resin by use of at least one Buffer I is selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl)piperazin-1-yl]ethanesulfonic acid (HEPES), sodium acetate, potassium acetate, and mixtures thereof. In a specific facet, the at least one Buffer I is selected from the group consisting of sodium phosphate solutions, potassium phosphate solutions, and mixtures thereof. In specific facets of this occurrence, the at least one Buffer I is provided in an amount sufficient to adjust the pH to a range of from about 6 to about 10, such as an amount sufficient to adjust the pH to a range of from about 6.8 to about 8.2.

In facets of the third occurrence of this fifth instance, the at least one adenylate kinase type enzyme is immobilized on the at least one hydrophilic resin by dissolving a lyophilized powder of the at least one acetate kinase type enzyme in at least one Buffer I, at a concentration of from about 1 g/l to about 100 g/l. In particular examples of these facets, the at least one adenylate kinase type enzyme at a concentration of from about 5 g/l to about 25 g/l. In additional examples of these facets, the at least one adenylate kinase type enzyme is 2 or more enzymes which are dissolved in a single vessel, in a gravimetric ratio of from about 1:1 to about 1:100, such as from about 1:1 to about 1:10. In specific examples of these facets, the total concentration of the one or more adenylate kinase type enzyme is from about 1 g/l to about 100 g/l, such as from about 5 g/l to about 25 g/l.

In a fourth occurrence of this fifth instance, the at least one adenylate kinase type enzyme is immobilized on the at least one hydrophilic resin in the presence of imidazole, NaCl, or mixtures thereof. In facets of this occurrence, the imidazole is present in an amount of from about 0 mM to about 30 mM, and the NaCl is present in an amount of about 500 mM.

In a fifth occurrence of this fifth instance, the at least one adenylate kinase type enzyme is immobilized on the at least one hydrophilic resin by incubating the one or more adenylate kinase type enzyme and the at least one hydrophilic resin in an agitated vessel at a temperature of from about 2° C. to about 30° C. over a period of from 10 minutes to 1 week. In specific facets of this occurrence, incubating is in an agitated vessel at a temperature of from about 2° C. to about 6° C. over a period of from 2 hours and 2 days. In other specific facets of this occurrence, incubating is in an agitated vessel at a temperature of from about 20° C. to about 25° C. over a period of from 30 minutes and 1 day.

In a sixth occurrence of this fifth instance, the at least one adenylate kinase type enzyme is immobilized on the at least one hydrophilic resin by passing a solution containing the at least one adenylate kinase type enzyme through a packed bed reactor containing the at least one hydrophilic resin dissolved in a Buffer K. In facets of this occurrence, the Buffer K is selected from the group consisting of sodium phosphate solutions, potassium phosphate solutions, and mixtures thereof. In specific facets of this occurrence, the at least one Buffer K is provided in an amount sufficient to adjust the pH to a range of from about 6 to about 10, such as an amount sufficient to adjust the pH to a range of from about 6.8 to about 8.2. In additional facets of this occurrence, the at least one Buffer K comprises imidazole, NaCl, or mixtures thereof.

In a seventh occurrence of this fifth instance, the at least one hydrophilic resin is clarified by washing with a Buffer L having a pH in a range of from about 6.8 to about 8.5 and comprising one or more reagent selected from the group consisting of sodium phosphate solutions, potassium phosphate solutions, 2-amino-2-(hydroxymethyl)propane-1,3-diol (Tris), bis-(2-hydroxyethyl) amino-tris(hydroxymethyl) methane (bis-Tris), 2-[4-(2-hydroxyethyl) piperazin-1-yl]ethanesulfonic acid (HEPES), sodium acetate, potassium acetate, NaCl, and imidazole, and mixtures thereof. In facets of this seventh occurrence, the Buffer L comprises NaCl, imidazole, or mixtures thereof, such as mixtures of from about OM to about 1M NaCl and of from about 0 mM to about 300 mM imidazole. In further facets of this seventh occurrence, the Buffer L comprises sodium phosphate, NaCl, imidazole, or mixtures thereof, such as mixtures of from about 50 mM to about 100 mM sodium phosphate, of from about 300 mM to about 500 mM NaCl and of from about 0 mM to about 50 mM imidazole. In still further facets of this seventh occurrence, the Buffer L comprises potassium phosphate, NaCl, imidazole, or mixtures thereof, such as mixtures of from about 50 mM to about 100 mM potassium phosphate, of from about 300 mM to about 500 mM NaCl and of from about 0 mM to about 50 mM imidazole. In facets of this eighth occurrence, the Buffer L has a concentration of from about 0 mM to about 100 mM.

In additional aspects, the at least one acetate kinase type enzyme and the at least one adenylate kinase type enzyme may be co-immobilized.

In a third aspect of the third embodiment, the reacting is conducted in the presence of a Co-Factor B. In instances of this aspect, the Co-Factor B is 2′F-thio-ATP or natural ATP. In specific instances of this aspect, the Co-Factor B is provided in an amount in a range of from about 0.02 to 0.1 equivalents with respect to the amount of the compound of Formula (I-2a).

In a fourth aspect of the third embodiment, the reacting is conducted in the presence of water. In specific occurrences of this instance, water is provided in an amount in a range of from about 20 to 50 volumes with respect to the amount of the compound of Formula (I-2a).

In a fifth aspect of the third embodiment, the reacting is conducted in the presence of a Metal Salt A. In instances of this aspect, the Metal Salt A is selected from the group consisting of divalent metal salts, hydrates thereof, and mixtures thereof. In specific instances, the at least one Metal Salt A is MgCl₂·(H₂O)₆. In specific instances of this aspect, the Metal Salt A is provided in an amount in a range of from about 0.125 to 1.5 equivalents with respect to the amount of the compound of Formula (I-2a).

In a sixth aspect of the third embodiment, the reacting is conducted in a temperature range of from about 5° C. to about 30° C. In instances of this aspect, the reacting is conducted in a temperature range of from about 10° C. to about 25° C. In specific instances, the reacting is conducted at a temperature of about 10° C. In instances of this aspect, the reacting is conducted over a time period in a range of about 10 h to about 100 h, such as over a time period in a range of about 20 h to about 80 h, over a time period in a range of about 30 h to about 50 h, over a time period of about 40 h.

In a seventh aspect of the third embodiment, the reacting further comprises forming a salt of the compound of Formula (I-2) by reacting the compound of Formula (I-2) with at least one Salt B is selected from the group consisting of magnesium salts, sodium salts, hydrates thereof, and mixtures thereof, to form a magnesium or sodium salt.

In a first instance of the seventh aspect, the compound of Formula (I-2) is acidified to a pH in a range of from about 2 to about 5. In occurrences of this instance, the compound of Formula (I-2) is acidified with HCl.

In a second instance of the seventh aspect, the at least one Salt B is selected from the group consisting of NaCl, MgCl₂, hydrates thereof, and mixtures thereof. In specific instances of this aspect, the at least one Salt B is MgCl₂·(H₂O)₆. In specific instances of this aspect, the at least one Salt B is provided in an amount in a range of from about 0 to 4.0 equivalents with respect to the amount of the compound of Formula (I-2).

In a third instance of the seventh aspect, the reacting further comprises crystallizing the salt of the compound of Formula (I-2). In occurrences of this instance, the crystallizing is conducted by addition of at least one Alcohol Solvent A. In specific occurrences, the at least one Alcohol Solvent A is selected from the group consisting of MeOH, EtOH, and IPA, and mixtures thereof. In more specific occurrences, the at least one Alcohol Solvent A is EtOH. In specific instances, the at least one Alcohol Solvent A is added in an amount in a range of from about 40% to about 50% of the total solvent volume. In occurrences of the third instance, the crystallizing comprises seeding with the compound of Formula (I-2). In specific occurrences, the crystallizing comprises adding the at least one Alcohol Solvent A in a 2:1 ratio of Alcohol Solvent A to water, and that the mixture is cooled to about 4° C. and filtered.

In a fourth embodiment, the process of the first embodiment further comprises simultaneously preparing the compound of Formula (I-1) by (i) reacting a compound of Formula (I-1a) with at least one guanylate kinase type enzyme and at least one acetate kinase enzyme, as described above in the second embodiment including all above-described aspects, and preparing the compound of Formula (I-2) by (ii) reacting a compound of Formula (I-2a) with at least one acetate kinase type enzyme and at least one adenylate kinase enzyme, as described above in the third embodiment including all above-described aspects.

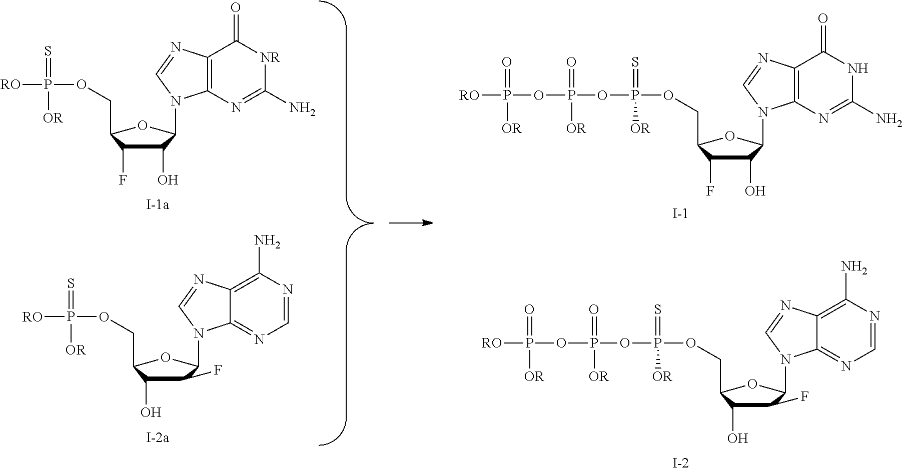

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In aspects of the fourth embodiment, the process is conducted in a single reaction vessel. In other aspects, the process is conducted in separate reaction vessels.

In a first aspect of the fourth embodiment, the at least one guanylate kinase type enzyme is selected independently from the group consisting of wild-type guanylate kinase type enzymes and guanylate kinase enzymes that are the product of directed evolution from a wild-type guanylate kinase type enzyme. In specific instances, the at least one guanylate kinase type enzyme is the wild-type guanylate kinase type enzyme having the amino acid sequence that is SEQ ID NO: 14. In a first instance of this first aspect, the at least one guanylate kinase type enzyme has the amino acid sequence that is SEQ ID NO: 15. In a second instance of this first aspect, the at least one guanylate kinase type enzyme has the amino acid sequence that is SEQ ID NO: 16. In a third instance of this first aspect, the at least one guanylate kinase type enzyme has the amino acid sequence that is SEQ ID NO: 17. In a fourth instance of this first aspect, the at least one guanylate kinase type enzyme has the amino acid sequence that is SEQ ID NO: 18. In a fifth instance of this first aspect, the at least one guanylate kinase type enzyme has the amino acid sequence that is SEQ ID NO: 19.

In even more specific instances of this aspect, the at least one guanylate kinase type enzyme is selected from wild-type guanylate kinase type enzyme, which has the amino acid sequence that is SEQ ID NO: 14, and guanylate kinase type enzymes that are the product of directed evolution from a wild-type guanylate kinase type enzyme and that have the amino acid sequence that is SEQ ID NO: 15, SEQ ID NO: 16, SEQ ID NO: 17, SEQ ID NO: 18, and SEQ ID NO: 19.

In a sixth instance of this first aspect, the at least one guanylate kinase type enzyme is selected from the group consisting of guanylate kinase type enzymes and immobilized guanylate kinase type enzymes, as described above with respect to the second embodiment.

In a second aspect of the fourth embodiment, the at least one acetate kinase type enzyme is selected independently from the group consisting of wild-type acetate kinase type enzymes and acetate kinase enzymes that are the product of directed evolution from a wild-type acetate kinase type enzyme. In specific instances, the at least one acetate kinase type enzyme is the wild-type acetate kinase type enzyme having the amino acid sequence that is SEQ ID NO: 20. In a first instance of this second aspect, the at least one acetate kinase type enzyme has the amino acid sequence that is SEQ ID NO: 21. In a second instance of this second aspect, the at least one acetate kinase type enzyme has the amino acid sequence that is SEQ ID NO: 22.

In specific instances of this aspect, the at least one acetate kinase type enzyme is selected from wild-type acetate kinase type enzyme, which has the amino acid sequence that is SEQ ID NO: 20, and acetate kinase type enzymes that are the product of directed evolution from a wild-type acetate kinase type enzyme and that have the amino acid sequence that is SEQ ID NO: 21 and SEQ ID NO: 22.

In a third instance of the second aspect of the fourth embodiment, each at least one acetate kinase enzyme is selected independently. In instances of these aspects, the at least one acetate kinase enzymes are different. In instances of these aspects, the at least one acetate kinase enzymes are the same.

In a second instance of this second aspect, the at least one acetate kinase type enzyme is selected from the group consisting of acetate kinase type enzymes and immobilized acetate kinase type enzymes, as described above with respect to the second and third embodiments.

In additional aspects, the at least one guanylate kinase type enzyme and the at least one acetate kinase type enzyme may be co-immobilized.

In a third aspect of the fourth embodiment, the (i) reacting is conducted in the presence of at least one Co-Factor A, as described above. In instances of this aspect, the at least one Co-Factor A is 2′F-thio-ATP or natural ATP. In specific instances of this aspect, the at least one Co-Factor A is provided in an amount in a range of from about 0.0005 to 2.0 equivalents with respect to the amount of the compound of Formula (I-1a).

In a fourth aspect of the fourth embodiment, the (i) reacting is conducted in the presence of at least one Metal Co-Factor B, as described above. In instances of this aspect, the at least one Metal Co-Factor B is selected from the group consisting of MgCl₂, MnCl₂, and Mg(OH)₂, hydrates thereof, and mixtures thereof. In specific instances, the at least one Metal Co-Factor B is MgCl₂. In specific instances of this aspect, the at least one Metal Co-Factor B is provided in an amount in a range of from about 0.1 to 5.0 equivalents with respect to the amount of the compound of Formula (I-1a).

In a fifth aspect of the fourth embodiment, the (i) reacting is conducted in the presence of at least one Inorganic Salt D selected from the group consisting of KCl, KBr, and NaCl, and mixtures thereof, as described above. In specific instances of this aspect, the at least one Inorganic Salt D is KCl. In specific instances of this aspect, the at least one Inorganic Salt D is provided in an amount in a range of from about 0.1 to 10.0 equivalents with respect to the amount of the compound of Formula (I-1a).

In a fifth aspect of the fourth embodiment, the (i) reacting is conducted in the presence of at least one Salt A selected from the group consisting of AcP—Li/Li, AcP—Na/Na, AcP—K/K, AcP—Li/K, AcP—NH₄/NH₄, and mixtures thereof, as described above. In specific instances of this aspect, the at least one Salt A is AcP—Li/Li. In specific instances of this aspect, the at least one Salt A is provided in an amount in a range of from about 0.5 to 7.0 equivalents with respect to the amount of the compound of Formula (I-1a).

In a sixth aspect of the fourth embodiment, the (i) reacting is conducted in the presence of at least one Solvent B, as described above.

In a seventh aspect of the fourth embodiment, the (i) reacting is conducted in a temperature range of from about −10° C. to about 35° C., as described above. In instances of this aspect, the reacting is conducted in a temperature range of from about 0° C. to about 25° C.

In an eighth aspect of the fourth embodiment, the (i) reacting is conducted in the presence of Base C, which is selected from the group consisting of KOH, NaOH, and mixtures thereof, as described above. In specific instances, Base C is KOH. In other specific instances, Base C is NaOH. In instances of this embodiment, Base C is included in an amount sufficient to control pH in a range of from about 5.5 to about 8.5. In instances of this embodiment, Base C is included in an amount sufficient to control pH in a range of from about 6.4 to about 7.0 at a temperature of about 25° C.

In a ninth aspect of the fourth embodiment, the at least one adenylate kinase enzyme is selected independently from the group consisting of wild-type adenylate kinase type enzymes and adenylate kinase enzymes that are the product of directed evolution from a wild-type adenylate kinase type enzyme. In specific instances, the at least one adenylate kinase type enzyme is the wild-type adenylate kinase type enzyme having the amino acid sequence that is SEQ ID NO: 23. In a first instance of this ninth aspect, the at least one adenylate kinase type enzyme has the amino acid sequence that is SEQ ID NO: 24. In a second instance of this ninth aspect, the at least one adenylate kinase type enzyme has the amino acid sequence that is SEQ ID NO: 25. In a third instance of this ninth aspect, the at least one adenylate kinase type enzyme has the amino acid sequence that is SEQ ID NO: 26. In a fourth instance of this ninth aspect, the at least one adenylate kinase type enzyme has the amino acid sequence that is SEQ ID NO: 27.

In even more specific instances of this ninth aspect, the at least one adenylate kinase type enzyme is selected from wild-type adenylate kinase type enzyme, which has the amino acid sequence that is SEQ ID NO: 23, and adenylate kinase type enzymes that are the product of directed evolution from a wild-type adenylate kinase type enzyme and that have the amino acid sequence that is SEQ ID NO: 24, SEQ ID NO: 25, SEQ ID NO: 26, and SEQ ID NO: 27.

In instances of this ninth aspect, the at least one adenylate kinase type enzyme is selected from the group consisting of adenylate kinase type enzymes and immobilized adenylate kinase type enzymes, as described above.

In additional aspects, the at least one acetate kinase type enzyme and the at least one adenylate kinase type enzyme may be co-immobilized.

In still further aspects, two or three of the at least one guanylate kinase type enzyme, the at least one acetate kinase type enzyme, and the at least one adenylate kinase type enzyme may be co-immobilized.

In a tenth aspect of the fourth embodiment, the (ii) reacting is conducted in the presence of a Co-Factor B. In instances of this aspect, the Co-Factor B is 2′F-thio-ATP or natural ATP. In specific instances of this aspect, the Co-Factor B is provided in an amount in a range of from about 0.02 to 0.1 equivalents with respect to the amount of the compound of Formula (I-2a).

In an eleventh aspect of the fourth embodiment, the (ii) reacting is conducted in the presence of water. In specific instances, water is provided in an amount in a range of from about 20 to 50 volumes with respect to the amount of the compound of Formula (I-2a).

In a twelfth aspect of the fourth embodiment, the (ii) reacting is conducted in the presence of a Metal Salt A. In instances of this aspect, the Metal Salt A is selected from the group consisting of divalent metal salts, hydrates thereof, and mixtures thereof. In specific instances, the at least one Metal Salt A is MgCl₂·(H₂O)₆. In specific instances of this aspect, the Metal Salt A is provided in an amount in a range of from about 0.125 to 1.5 equivalents with respect to the amount of the compound of Formula (I-2a).

In a thirteenth aspect of the fourth embodiment, the (ii) reacting is conducted in a temperature range of from about 5° C. to about 30° C. In instances of this aspect, the reacting is conducted in a temperature range of from about 10° C. to about 25° C. In specific instances, the reacting is conducted at a temperature of about 10° C. In instances of this aspect, the reacting is conducted over a time period in a range of about 10 h to about 100 h, such as over a time period in a range of about 20 h to about 80 h, over a time period in a range of about 30 h to about 50 h, over a time period of about 40 h.

In a fifteenth aspect of the fourth embodiment, the (ii) reacting further comprises forming a salt of the compound of Formula (I-2) by reacting the compound of Formula (I-2) with at least one Salt B is selected from the group consisting of magnesium salts, sodium salts, hydrates thereof, and mixtures thereof, to form a magnesium or sodium salt.

In a first instance of the fifteenth aspect, the compound of Formula (I-2) is acidified to a pH in a range of from about 2 to about 5. In occurrences of this instance, the compound of Formula (I-2) is acidified with HCl.

In a second instance of the fifteenth aspect, the at least one Salt B is selected from the group consisting of NaCl, MgCl₂, hydrates thereof, and mixtures thereof. In specific instances of this aspect, the at least one Salt B is MgCl₂·(H₂O)₆. In specific instances of this aspect, the at least one Salt B is provided in an amount in a range of from about 0 to 4.0 equivalents with respect to the amount of the compound of Formula (I-2).

In a third instance of the fifteenth aspect, the reacting further comprises crystallizing the salt of the compound of Formula (I-2). In occurrences of this instance, the crystallizing is conducted by addition of at least one Alcohol Solvent A. In specific occurrences, the at least one Alcohol Solvent A is selected from the group consisting of MeOH, EtOH, and IPA, and mixtures thereof. In more specific occurrences, the at least one Alcohol Solvent A is EtOH. In specific instances, the at least one Alcohol Solvent A is added in an amount in a range of from about 40% to about 50% of the total solvent volume. In occurrences of the third instance, the crystallizing comprises seeding with the compound of Formula (I-2). In specific occurrences, the crystallizing comprises adding the at least one Alcohol Solvent A in a 2:1 ratio of Alcohol Solvent A to water, and that the mixture is cooled to about 4° C. and filtered.

A fifth embodiment relates to a process for preparing a compound of Formula (I), which comprises preparing the compound of Formula (I-1) by (i) reacting a compound of Formula (I-la) with at least one guanylate kinase type enzyme and at least one acetate kinase enzyme, as described above in the second embodiment including all above-described aspects, and preparing the compound of Formula (I-2) by (ii) reacting a compound of Formula (I-2a) with at least one acetate kinase type enzyme and at least one adenylate kinase enzyme, followed by (iii) reacting a compound of Formula (I-1) with a compound of Formula (I-2), in the presence of at least one cyclic GMP-AMP synthase (cGAS) type enzyme, as described in the first through fifth embodiments:

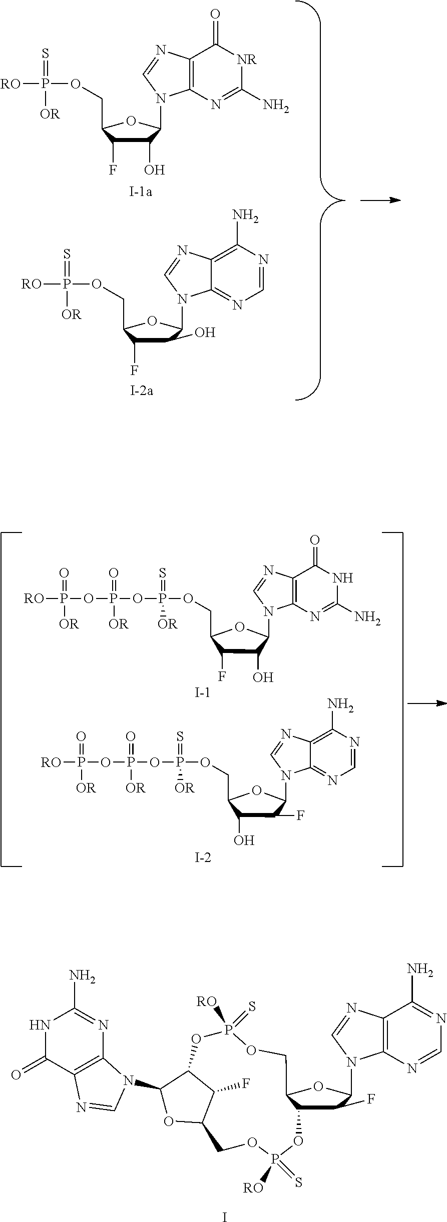

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In aspects of the fifth embodiment, the process is conducted in a single reaction vessel. In other aspects, the process is conducted in separate reaction vessels.

In a first aspect of the fifth embodiment, the at least one guanylate kinase type enzyme, as described above, is selected independently from the group consisting of wild-type guanylate kinase type enzymes and guanylate kinase enzymes that are the product of directed evolution from a wild-type guanylate kinase type enzyme. In specific instances, the at least one guanylate kinase type enzyme is the wild-type guanylate kinase type enzyme having the amino acid sequence that is SEQ ID NO: 14. In a first instance of this first aspect, the at least one guanylate kinase type enzyme has the amino acid sequence that is SEQ ID NO: 15. In a second instance of this first aspect, the at least one guanylate kinase type enzyme has the amino acid sequence that is SEQ ID NO: 16. In a third instance of this first aspect, the at least one guanylate kinase type enzyme has the amino acid sequence that is SEQ ID NO: 17. In a fourth instance of this first aspect, the at least one guanylate kinase type enzyme has the amino acid sequence that is SEQ ID NO: 18. In a fifth instance of this first aspect, the at least one guanylate kinase type enzyme has the amino acid sequence that is SEQ ID NO: 19.

In even more specific instances of this aspect, the at least one guanylate kinase type enzyme is selected from wild-type guanylate kinase type enzyme, which has the amino acid sequence that is SEQ ID NO: 14, and guanylate kinase type enzymes that are the product of directed evolution from a wild-type guanylate kinase type enzyme and that have the amino acid sequence that is SEQ ID NO: 15, SEQ ID NO: 16, SEQ ID NO: 17, SEQ ID NO: 18, and SEQ ID NO: 19.

In a sixth instance of this first aspect, the at least one guanylate kinase type enzyme is selected from the group consisting of guanylate kinase type enzymes and immobilized guanylate kinase type enzymes, as described above with respect to the second embodiment.

In a second aspect of the fifth embodiment, the at least one acetate kinase type enzyme, as described above, is selected independently from the group consisting of wild-type acetate kinase type enzymes and acetate kinase enzymes that are the product of directed evolution from a wild-type acetate kinase type enzyme. In specific instances, the at least one acetate kinase type enzyme is the wild-type acetate kinase type enzyme having the amino acid sequence that is SEQ ID NO: 20. In a first instance of this second aspect, the at least one acetate kinase type enzyme has the amino acid sequence that is SEQ ID NO: 21. In a second instance of this second aspect, the at least one acetate kinase type enzyme has the amino acid sequence that is SEQ ID NO: 22.

In specific instances of this aspect, the at least one acetate kinase type enzyme is selected from wild-type acetate kinase type enzyme, which has the amino acid sequence that is SEQ ID NO: 20, and acetate kinase type enzymes that are the product of directed evolution from a wild-type acetate kinase type enzyme and that have the amino acid sequence that is SEQ ID NO: 21 and SEQ ID NO: 22.

In a third instance of the second aspect of the fifth embodiment, each at least one acetate kinase enzyme is selected independently. In instances of these aspects, the at least one acetate kinase enzymes are different. In instances of these aspects, the at least one acetate kinase enzymes are the same.

In a second instance of this second aspect, the at least one acetate kinase type enzyme is selected from the group consisting of acetate kinase type enzymes and immobilized acetate kinase type enzymes, as described above with respect to the second and third embodiments.

In additional aspects, the at least one guanylate kinase type enzyme and the at least one acetate kinase type enzyme may be co-immobilized.

In a third aspect of the fifth embodiment, the (i) reacting is conducted in the presence of at least one Co-Factor A, as described above. In instances of this aspect, the at least one Co-Factor A is 2′F-thio-ATP or natural ATP. In specific instances of this aspect, the at least one Co-Factor A is provided in an amount in a range of from about 0.00002 to 2.0 equivalents with respect to the amount of the compound of Formula (I-1a). In more specific instances of this aspect, the at least one Co-Factor A is provided in an amount in a range of from about 0.0001 to 2.0 equivalents with respect to the amount of the compound of Formula (I-1a). In still more specific instances of this aspect, the at least one Co-Factor A is provided in an amount in a range of from about 0.00022 to 2.0 equivalents with respect to the amount of the compound of Formula (I-1a). In additional specific instances of this aspect, the at least one Co-Factor A is provided in an amount in a range of from about 0.0005 to 2.0 equivalents with respect to the amount of the compound of Formula (I-1a). In certain instances, the at least on Co-Factor A is provided in an amount of about 0.0001 equivalents with respect to the amount of the compound of Formula (I-la). In certain instances, the at least on Co-Factor A is provided in an amount of about 0.0002 equivalents with respect to the amount of the compound of Formula (I-1a). In certain instances, the at least on Co-Factor A is provided in an amount of about 0.0005 equivalents with respect to the amount of the compound of Formula (I-1a).

In a fourth aspect of the fifth embodiment, the (i) reacting is conducted in the presence of at least one Metal Co-Factor B, as described above. In instances of this aspect, the at least one Metal Co-Factor B is selected from the group consisting of MgCl₂, MnCl₂, and Mg(OH)₂, hydrates thereof, and mixtures thereof. In specific instances, the at least one Metal Co-Factor B is MgCl₂. In specific instances of this aspect, the at least one Metal Co-Factor B is provided in an amount in a range of from about 0.1 to 5.0 equivalents with respect to the amount of the compound of Formula (I-1a).

In a fifth aspect of the fifth embodiment, the (i) reacting is conducted in the presence of at least one Inorganic Salt D selected from the group consisting of KCl, KBr, and NaCl, and mixtures thereof, as described above. In specific instances of this aspect, the at least one Inorganic Salt D is KCl. In specific instances of this aspect, the at least one Inorganic Salt D is provided in an amount in a range of from about 0.1 to 10.0 equivalents with respect to the amount of the compound of Formula (I-1a).

In a fifth aspect of the fifth embodiment, the (i) reacting is conducted in the presence of at least one Salt A selected from the group consisting of AcP—Li/Li, AcP—Na/Na, AcP—K/K, AcP—Li/K, AcP—NH₄/NH₄, and mixtures thereof, as described above. In specific instances of this aspect, the at least one Salt A is AcP—Li/Li. In specific instances of this aspect, the at least one Salt A is provided in an amount in a range of from about 0.5 to 7.0 equivalents with respect to the amount of the compound of Formula (I-1a).

In a sixth aspect of the fifth embodiment, the (i) reacting is conducted in the presence of at least one Solvent B, as described above.

In a seventh aspect of the fifth embodiment, the (i) reacting is conducted in a temperature range of from about −10° C. to about 35° C., as described above. In instances of this aspect, the reacting is conducted in a temperature range of from about 0° C. to about 25° C.

In an eighth aspect of the fifth embodiment, the (i) reacting is conducted in the presence of Base C, which is selected from the group consisting of KOH, NaOH, and mixtures thereof, as described above. In specific instances, Base C is KOH. In other specific instances, Base C is NaOH. In instances of this embodiment, Base C is included in an amount sufficient to control pH in a range of from about 5.5 to about 8.5. In instances of this embodiment, Base C is included in an amount sufficient to control pH in a range of from about 6.4 to about 7.0 at a temperature of about 25° C.

In a ninth aspect of the fifth embodiment, the at least one adenylate kinase enzyme is selected independently from the group consisting of wild-type adenylate kinase type enzymes and adenylate kinase enzymes that are the product of directed evolution from a wild-type adenylate kinase type enzyme. In specific instances, the at least one adenylate kinase type enzyme is the wild-type adenylate kinase type enzyme having the amino acid sequence that is SEQ ID NO: 23. In a first instance of this ninth aspect, the at least one adenylate kinase type enzyme has the amino acid sequence that is SEQ ID NO: 24. In a second instance of this ninth aspect, the at least one adenylate kinase type enzyme has the amino acid sequence that is SEQ ID NO: 25. In a third instance of this ninth aspect, the at least one adenylate kinase type enzyme has the amino acid sequence that is SEQ ID NO: 26. In a fourth instance of this ninth aspect, the at least one adenylate kinase type enzyme has the amino acid sequence that is SEQ ID NO: 27.

In even more specific instances of this ninth aspect, the at least one adenylate kinase type enzyme is selected from wild-type adenylate kinase type enzyme, which has the amino acid sequence that is SEQ ID NO: 23, and adenylate kinase type enzymes that are the product of directed evolution from a wild-type adenylate kinase type enzyme and that have the amino acid sequence that is SEQ ID NO: 24, SEQ ID NO: 25, SEQ ID NO: 26, and SEQ ID NO: 27.

In instances of this ninth aspect, the at least one adenylate kinase type enzyme is selected from the group consisting of adenylate kinase type enzymes and immobilized adenylate kinase type enzymes, as described above.

In additional aspects, the at least one acetate kinase type enzyme and the at least one adenylate kinase type enzyme may be co-immobilized.

In particular additional aspects, at least two of the at least one guanylate kinase type enzyme, the at least one acetate kinase type enzyme, and the at least one adenylate kinase type enzyme may be co-immobilized. In more particular additional aspects, at least three of the at least one guanylate kinase type enzyme, the at least one acetate kinase type enzyme, and the at least one adenylate kinase type enzyme may be co-immobilized.

In a tenth aspect of the fifth embodiment, the (ii) reacting is conducted in the presence of a Co-Factor B, as described above. In instances of this aspect, the Co-Factor B is 2′F-thio-ATP or natural ATP. In specific instances of this aspect, the Co-Factor B is provided in an amount in a range of from about 0.02 to 0.1 equivalents with respect to the amount of the compound of Formula (I-2a).

In an eleventh aspect of the fifth embodiment, the (ii) reacting is conducted in the presence of water, as described above. In specific instances, water is provided in an amount in a range of from about 20 to 500 volumes with respect to the amount of the compound of Formula (I-2a). In more specific instances, water is provided in an amount in a range of from about 20 to 200 volumes with respect to the amount of the compound of Formula (I-2a). In still more specific instances, water is provided in an amount in a range of from about 20 to 50 volumes with respect to the amount of the compound of Formula (I-2a).

In a twelfth aspect of the fifth embodiment, the (ii) reacting is conducted in the presence of a Metal Salt A, as described above. In instances of this aspect, the Metal Salt A is selected from the group consisting of divalent metal salts, hydrates thereof, and mixtures thereof. In specific instances, the at least one Metal Salt A is MgCl₂·(H₂O)₆. In specific instances of this aspect, the Metal Salt A is provided in an amount in a range of from about 0.125 to 1.5 equivalents with respect to the amount of the compound of Formula (I-2a).

In a thirteenth aspect of the fifth embodiment, the (ii) reacting is conducted in a temperature range of from about 5° C. to about 30° C., as described above. In instances of this aspect, the reacting is conducted in a temperature range of from about 10° C. to about 25° C. In specific instances, the reacting is conducted at a temperature of about 10° C. In instances of this aspect, the reacting is conducted over a time period in a range of about 10 h to about 100 h, such as over a time period in a range of about 20 h to about 80 h, over a time period in a range of about 30 h to about 50 h, over a time period of about 40 h.

In a fifteenth aspect of the fifth embodiment, the (ii) reacting further comprises forming a salt of the compound of Formula (I-2) by reacting the compound of Formula (I-2) with at least one Salt B is selected from the group consisting of magnesium salts, sodium salts, hydrates thereof, and mixtures thereof, to form a magnesium or sodium salt, as described above.

In a first instance of the fifteenth aspect, the compound of Formula (I-2) is acidified to a pH in a range of from about 2 to about 5, as described above. In occurrences of this instance, the compound of Formula (I-2) is acidified with HCl.

In a second instance of the fifteenth aspect, the at least one Salt B, as described above, is selected from the group consisting of NaCl, MgCl₂, hydrates thereof, and mixtures thereof. In specific instances of this aspect, the at least one Salt B is MgCl₂·(H₂O)₆. In specific instances of this aspect, the at least one Salt B is provided in an amount in a range of from about 0 to 4.0 equivalents with respect to the amount of the compound of Formula (I-2).

In a third instance of the fifteenth aspect, the (ii) reacting further comprises crystallizing the salt of the compound of Formula (I-2), as described above. In occurrences of this instance, the crystallizing is conducted by addition of at least one Alcohol Solvent A. In specific occurrences, the at least one Alcohol Solvent A is selected from the group consisting of MeOH, EtOH, and IPA, and mixtures thereof. In more specific occurrences, the at least one Alcohol Solvent A is EtOH. In specific instances, the at least one Alcohol Solvent A is added in an amount in a range of from about 40% to about 50% of the total solvent volume. In occurrences of the third instance, the crystallizing comprises seeding with the compound of Formula (I-2). In specific occurrences, the crystallizing comprises adding the at least one Alcohol Solvent A in a 2:1 ratio of Alcohol Solvent A to water, and that the mixture is cooled to about 4° C. and filtered.

In a sixteenth aspect of the fifth embodiment, as described above, the compound of Formula (I-1) and compound of Formula (I-2) are provided in a ratio of from about 10:1, from about 7:1, from about 5:1, from about 4:1, from about 2:1, from about 1:1, from about 1:2, from about 1:4, from about 1:5, from about 1:7, or from about 1:10.

In a seventeenth aspect of the fifth embodiment, the at least one cyclic GMP-AMP synthase (cGAS) type enzyme is selected from the group consisting of wild-type cGAS type enzymes and cGAS type enzymes that are the product of directed evolution from a wild-type cGAS type enzyme, which has the amino acid sequence that is SEQ ID NO: 1. In a first instance of this seventeenth aspect, the at least one cGAS type enzyme has the amino acid sequence that is SEQ ID NO: 2. In a second instance of this seventeenth aspect, the at least one cGAS type enzyme has the amino acid sequence that is SEQ ID NO: 3. In a third instance of this seventeenth aspect, the at least one cGAS type enzyme has the amino acid sequence that is SEQ ID NO: 4. In a fourth instance of this seventeenth aspect, the at least one cGAS type enzyme has the amino acid sequence that is SEQ ID NO: 5. In a fifth instance of this seventeenth aspect, the at least one cGAS type enzyme has the amino acid sequence that is SEQ ID NO: 6. In a sixth instance of this seventeenth aspect, the at least one cGAS type enzyme has the amino acid sequence that is SEQ ID NO: 7. In a seventh instance of this seventeenth aspect, the at least one cGAS type enzyme has the amino acid sequence that is SEQ ID NO: 8. In an eighth instance of this seventeenth aspect, the at least one cGAS type enzyme has the amino acid sequence that is SEQ ID NO: 9. In a ninth instance of this seventeenth aspect, the at least one cGAS type enzyme has the amino acid sequence that is SEQ ID NO: 10. In a tenth instance of this seventeenth aspect, the at least one cGAS type enzyme has the amino acid sequence that is SEQ ID NO: 11. In an eleventh instance of this seventeenth aspect, the at least one cGAS type enzyme has the amino acid sequence that is SEQ ID NO: 12. In a twelfth instance of this seventeenth aspect, the at least one cGAS type enzyme has the amino acid sequence that is SEQ ID NO: 13.

In an eighteenth aspect of the fifth embodiment, the at least one cGAS type enzyme is provided in an amount in a range of from about 1.0 percent by weight (wt %) to about 100.0 wt % with respect to the amount of the compound of Formula (I-1), such as an amount in a range of from about 10.0 wt % to about 50 wt %, or an amount in a range of from about 20 wt % to about 40 wt %.

In a nineteenth aspect of the fifth embodiment, the at least one cGAS type enzyme can be used as the whole cell lysate, a cGAS wet pellet, a purified cGAS wet pellet, a Co-treated cGAS wet pellet, or as a lyophilized powder.

In a twentieth aspect of the fifth embodiment, as described above, the at least one cGAS type enzyme can be incubated in at least one Chaotropic Agent A including, but not limited to, sodium dodecyl sulfate (SDS), thiourea, guanidine HCl, phenol, phenyl acetyl sulfide, urea, KCl, MgCl₂, LiOAc, NaCl, and mixtures thereof. In instances of this fifth aspect of the fifth embodiment, the at least one chaotropic agent is MgCl₂.

In a first instance of the twentieth aspect of the fifth embodiment, the at least one Chaotropic Agent A is provided in an amount in a range of from about 0.01M to 2M, such an amount in a range of from about 0.05M to 1M, or an amount in a range of from about 0.1M to 0.5M.

In a second instance of the twentieth aspect of the fifth embodiment, the at least one Chaotropic Agent A is provided in a range of from about 5 to about 10 volumes with respect to the amount of the at least one cGAS type enzyme, such an amount in a range of from about 3 to 5 volumes, or an amount of about 1 volume.

In a third instance of the twentieth aspect of the fifth embodiment, the at least one cGAS type enzyme is incubated in the at least one Chaotropic Agent A at a temperature range of 5° C. to 80° C., such as at a temperature in a range of from about 10° C. to about 50° C., or about 23° C.

In a fourth instance of the twentieth aspect of the fifth embodiment, the at least one cGAS type enzyme is incubated in the at least one Chaotropic Agent A at a pH range of 4 to 14, such as at a pH in a range of from about 6 to about 10, or about 8.

In a twenty-first aspect of the fifth embodiment, the reacting further comprises reacting in the presence of at least one Metal Co-Factor A, as described above. In instances of this twenty-first aspect, the at least one Metal Co-Factor A is selected from the group consisting of KCl, MgCl₂, ZnSO₄, CoSO₄, CoF₂, Co(SCN)₂, CoBr₂, Co(NO₃)₂, CoCl₂, CoCO₃, Co(C₂O₄)₂, and Co(OH)₂, and mixtures thereof. In specific instances, the at least one Metal Co-Factor A is CoSO₄.

In a first instance of this twenty-first aspect, the at least one Metal Co-Factor A is provided in an amount in a range of from about 0.01M to 2M, such an amount in a range of from about 0.05M to 1M, or an amount in a range of from about 0.1M to 0.5M.

In a second instance of this twenty-first aspect, the at least one Metal Co-Factor A is provided in a range of from about 5 to about 10 volumes with respect to the amount of the at least one cGAS type enzyme, such an amount in a range of from about 3 to 5 volumes, or an amount of about 1 volume.

In a third instance of this twenty-first aspect, the at least one cGAS type enzyme is incubated with the at least one Metal Co-Factor A.

In a first occurrence of this third instance, the at least one cGAS type enzyme is incubated with the at least one Metal Co-Factor A at a temperature range of 5° C. to 80° C., such as at a temperature in a range of from about 10° C. to about 50° C., or about 23° C.

In a second occurrence of this third instance, the at least one cGAS type enzyme is incubated with the at least one Metal Co-Factor A at a pH range of 4 to 14, such as at a pH in a range of from about 6 to about 10, or pH of about 8.

In a third occurrence of this third instance, the at least one cGAS type enzyme is incubated with the at least one Metal Co-Factor A in the presence of at least one Base A from the group consisting of TES, KOH, and NaOH, and mixtures thereof. In more specific occurrences, the at least one Base A is KOH.

In a twenty-second aspect of the fifth embodiment, the reacting is conducted in the presence of at least one Inorganic Salt A, as described above. In instances of the twenty-second aspect, the at least one Inorganic Salt A is selected from the group consisting of CoSO₄, ZnSO₄, CoCl₂, Co(acac)₂, ZnF₂, ZnCl₂, MoCl₅, SbCl₅, CuCl, CuCl₂, CuBr, CuBr₂, CuF₂, CuOAc, CuSO₄, Fe(II)BF₄, V(O)(acac)₂, Pt(II)Cl₂, Ho(OTf)₃, Ni(II)Br, La(acac)₃, CeCl₃, KBF₄, MgCl₂, Zn(OTf)₂, ZnBr₂, CoBr₂, Zn(OAc)₂, Co(OAc)₂, Mg(OH)₂, hydrates of the aforementioned, and mixtures thereof. In specific instances of this seventh aspect, the at least one Inorganic Salt A is selected from the group consisting of CoSO₄, ZnSO₄, CoCl₂, ZnCl₂, Zn(OTf)₂, Zn(OAc)₂, and mixtures thereof. In specific instances of this seventh aspect, the at least one Inorganic Salt A is a mixture selected from the group consisting of CoCl₂ and ZnCl₂, CoCl₂ and Zn(OAc)₂, CoSO₄ and ZnCl₂, CoSO₄ and Zn(OTf)₂, CoSO₄ and Zn(OAc)₂, and CoSO₄ and Zn(OTf)₂. In a specific instance of this aspect, the Inorganic Salt A is selected from the group consisting of CoSO₄ and ZnSO₄, and mixtures thereof.

In a first instance of this twenty-second aspect, the at least one Inorganic Salt A is provided in an amount in a range of from about 0.1 to about 5.0 equivalents with respect to the amount of the compound of Formula (I-1).

In a twenty-third aspect of the fifth embodiment, the at least one cGAS type enzyme has been isolated, as described above. In instances of this aspect, a crude lysate containing the at least one cGAS type enzyme is subjected to centrifugation, and the pellet fraction is slurried and incubated with an aqueous solution of at least one Inorganic Salt B consisting of Na₂SO₄, (NH₄)₂SO₄, NaCl, KCl, K₂SO₄, hydrates of the aforementioned, and mixtures thereof. In particular instances, the volume of the aqueous solution of at least one Inorganic Salt B can range from 0.1 volumes to 5 volumes relative to the initial volume of crude lysate. In additional instances, the concentration of this solution can range from 0.1M to 1.5M. In instances of this aspect, the slurry is subjected to centrifugation following incubation with the aqueous solution of at least one Inorganic Salt B, and the liquid fraction containing cGAS type enzyme is retained.

In a first occurrence of the instances of the twenty-third aspect, the concentration of at least one Inorganic Salt B is reduced in liquid fraction containing cGAS type enzyme, which may be accomplished by a method selected from the group consisting of dialysis, tangential flow filtration, dilution with water, and dilution with a solution comprising at least one Inorganic Salt C, which is selected from the group consisting of CoSO₄, CoCl₂, ZnSO₄, ZnCl₂, MgSO₄, and MgCl₂, hydrates of the aforementioned, and mixtures thereof. In particular facets of this occurrence, the solution comprising at least one Inorganic Salt C includes the at least one Inorganic Salt C at a concentration of 0.01M to 0.1M. In specific facets of this occurrence, the at least one cGAS type enzyme will precipitate from the solution having reduced salt concentration and can then be isolated by centrifugation.

In a second occurrence of the instances of the twenty-third aspect, the at least one cGAS type enzyme is purified by a chromatographic technique, as described above. In particular facets of this occurrence, the chromatographic technique is selected from the group consisting of immobilized metal-affinity chromatography, ion-exchange chromatography, hydrophobic interaction chromatography, and size-exclusion chromatography.

In occurrences of the instances of the twenty-third aspect, the isolated and/or purified at least one cGAS type enzyme may be activated via addition of deoxyribonucleic acid at a ratio between 0.1 to 1 gram of deoxyribonucleic acid to 1 gram of cGAS type enzyme.

In a twenty-fourth aspect of the fifth embodiment, the reacting is conducted in the presence of at least one Solvent A, as described above.

In a first instance of the twenty-fourth aspect, the at least one Solvent A is selected from the group consisting of organic solvents, organic solvents in combination with water, and mixtures thereof. In occurrences of this instance, the at least one Solvent A is selected from the group consisting of organic solvents in combination with water. In instances of this aspect, the at least one Solvent A is selected from the group consisting of tetraglyme dimethyl ether (TGDE), MeCN, MeOH, EtOH, DMSO, propyl nitrile, sulfolane, pyrrolidone, 2-ethoxyl acetate, cyclohexanol, methyl pentyl ketone, cyclohexanone, 1,2,3,4-tetrahydronaphthalene, pivolate methyl ester, 2-methyl-3-butene-2-ol, tert-butanol, DMF, tetra-methyl urea, tetramethylene sulfone (also sulfolane or 1λ6-thiolane-1,1-dione), N,N-diethyl acetamide, ethylene glycol, NMP, isopropyl alcohol, 1-methoxy-2-propyl acetate (MPA), and mixtures thereof. In a specific instance of this aspect, the at least one Solvent A is TGDE. In specific instances of this aspect, the at least one Solvent A is provided in an amount in a range of from about 10 to 50 volumes with respect to the amount of the compound of Formula (I-1).

In a second instance of the twenty-fourth aspect, the at least one Solvent A is water. In specific occurrences of this instance, water is provided in an amount in a range of from about 10 to 1500 volumes with respect to the amount of the compound of Formula (I-1). In specific occurrences of this instance, the reaction is conducted in 50-200 volumes with respect to the amount of the compound of Formula (I-1).

In a twenty-fifth aspect of the fifth embodiment, the (iii) reacting is conducted, as described above, in the presence of at least one Phosphatase Inhibitor A selected from the group consisting of Na₃VO₄, Na₂P₂O₇, (HOCH₂)₂CH—OP(O)(ONa), EDTA, Na₂WO₄, Na₂MO₄, NaF, KF, CsF, and mixtures thereof. In instances of this aspect, the at least one Phosphatase Inhibitor A is Na₃VO₄. In further instances of this aspect, the at least one Phosphatase Inhibitor A is provided in an amount in a range of from about 0.005 to 2.0 equivalents with respect to the amount of the compound of Formula (I-1).

In twenty-sixth aspect of the fifth embodiment, the (iii) reacting is conducted, as described above, in the presence of at least one Buffer A selected from the group consisting of 2-[[1,3-dihydroxy-2-(hydroxymethyl)propan-2-yl]amino]ethanesulfonic acid (TES), 2-amino-2-(hydroxymethyl) propane-1,3-diol (Tris), 2-[4-(2-hydroxyethyl)piperazin-1-yl]ethanesulfonic acid (HEPES), 3-[[1,3-dihydroxy-2-(hydroxymethyl)propan-2-yl]amino]-2-hydroxypropane-1-sulfonic acid (TAPSO), 2-morpholin-4-ylethanesulfonic acid (MES), and mixtures thereof. In a specific instance of this aspect, the at least one Buffer A is 2-[[1,3-dihydroxy-2-(hydroxymethyl)propan-2-yl]amino]ethanesulfonic acid (TES). In specific instances of this aspect, the at least one Buffer A is provided in an amount in a range of from about 0.1 to 30 equivalents with respect to the amount of the compound of Formula (I-1).

In a twenty-seventh aspect of the fifth embodiment, the (iii) reacting is conducted in the presence of Base B, as described above, which is selected from the group consisting of KOH, NaOH, CsOH, (NH)₄OH, and mixtures thereof. In specific instances, Base B is KOH. In instances of this embodiment, Base B is included in an amount sufficient to control pH in a range of from about 7.1 to about 7.7. In specific occurrences of this instance, Base B is KOH, and pH is about 7.4.

In a twenty-eighth aspect of the fifth embodiment, the (iii) reacting is conducted in a temperature range of from about 5° C. to about 50° C., as described above. In instances of this aspect, the reacting is conducted in a temperature range of from about 25° C. to about 40° C.

In a twenty-ninth aspect of the fifth embodiment, the process further comprises forming a compound of Formula (I), which is a salt of the compound of Formula (Ia). In instances of this aspect, the compound of Formula (I) is a sodium, potassium, magnesium, cobalt, zinc, or ammonium salt. In specific instances, the compound of Formula (I) is a sodium or potassium salt. In instances of this aspect, the compound of Formula (I) comprises a cation selected from the group consisting of Na⁺, K⁺, Mg⁺⁺, Co⁺⁺, Zn⁺⁺, and NH₄⁺. In instances of this aspect, the compound of Formula (I) comprises two cations independently selected from the group consisting of Na⁺, K⁺, and NH₄⁺. In more specific instances, the compound of Formula (I) is a disodium salt or a dipotassium salt. In even more specific instances, the compound of Formula (I) is a disodium salt.

In a sixth embodiment, the process of the fifth embodiment further comprises isolating the compound of Formula (I), in which the isolating comprises precipitation of the compound of Formula (I), such as by pH-swing precipitation. In aspects, the isolating comprises precipitation, washing, and drying, such as by humid drying.

In a first aspect of the sixth embodiment, the process comprises agitating the compound of Formula (I) with at least one Inorganic Salt E. In instances of this aspect, the at least one Inorganic Salt E is selected from the group consisting of Na₃PO₄, Na₂HPO₄, NaH₂PO₄, (NH₄)₂SO₄, and mixtures thereof. In specific instances, the at least one Inorganic Salt E is Na₂HPO₄. In instances of this aspect, the at least one Inorganic Salt E is present in an amount in a range of from about 1 to about 20 equivalents with respect to the amount of the compound of Formula (I).

In a second aspect of the sixth embodiment, the process further comprises filtering the agitated mixture. In instances of the second aspect, the filtering is through at least one Filtration Aid. In particular instances, the at least one Filtration Aid is selected from silica gel, cellulose, diatomaceous earth, and mixtures thereof. In specific instances of this aspect, the at least one Filtration Aid is diatomaceous earth. In further instances of this aspect, the Filtration Aid is provided in an amount of from about 100 wt % to about 500 wt % with respect to the amount of the compound of Formula (I).

In a third aspect of the sixth embodiment, the process further comprises agitating the filtrate with an Inorganic Salt F. In instances of this aspect, the at least one Inorganic Salt F is selected from the group consisting of NaHCO₃, Na₂HPO₄, NaH₂PO₄, (NH₄)₂SO₄, Na₂SO₃, NaHSO₃, Na₂CO₃, and K₂CO₃, and hydrates thereof, and mixtures thereof. In specific instances, the at least one Inorganic Salt D is Na₂CO₃. In instances of this aspect, the at least one Inorganic Salt F is present in an amount in a range of from about 100 wt % to about 5000 wt % with respect to the amount of the compound of Formula (I).

In a fourth aspect of the sixth embodiment, the process further comprises crystallizing the compound of Formula (I). In specific instances of this aspect, the process comprises crystalizing the compound of Formula (Ia). In instances of this aspect, the crystallizing is conducted by extracting the agitated filtrate, and mixing the organic layer with at least one Aqueous Acid A selected from the group consisting of HCl, H₂SO₄, TFA, H₃PO₄, MsOH, TfOH, and mixtures thereof. In a specific instance of this aspect, the at least one Aqueous Acid A is HCl. In specific instances of this aspect, the at least one Aqueous Acid A is provided in an amount in a range of from about 2 to about 50 equivalents with respect to the amount of the compound of Formula (I). In specific instances of this aspect, the at least one Aqueous Acid A is provided in an amount to provide a pH in a range of from about 0.5 to about 1.5.

In a fifth aspect of the sixth embodiment, the process comprises filtering and washing with at least one Aqueous Acid A selected from the group consisting of HCl, H₂SO₄, TFA, H₃PO₄, MsOH, TfOH, and mixtures thereof. In a specific instance of this aspect, the at least one Aqueous Acid A is HCl. In specific instances of this aspect, the at least one Aqueous Acid A is provided in a solution that is from about 0.01N to about 2N. In specific instances of this aspect, a solution of from about 0.01N to about 2N solution of the at least one Aqueous Acid A provided in an amount in a range of from about 2 to about 50 equivalents with respect to the amount of the compound of Formula (I). In specific instances of this aspect, a solution of from about 0.01N to about 2N solution of the at least one Aqueous Acid A provided in an amount to provide a pH in a range of from about 0.5 to about 1.5.

In a seventh embodiment, the process of the fifth embodiment further comprises isolating the compound of Formula (I), in which the isolating comprises extracting, washing, crystallizing, and drying the compound of Formula (I). In instances of this embodiment, the process comprises isolating the compound of Formula (I) wherein each R is H. In other instances, the process comprises isolating the compound of Formula (I) wherein each R is Na. In further instances, the process comprises isolating the compound of Formula (I) wherein each R is K.

In a first aspect of the seventh embodiment, the process comprises reacting the crude compound of Formula (I) with at least one Salt C selected from the group consisting of Na₂SO₄, NaHSO₄, Na₂CO₃, NaHCO₃, K₂SO₄, KHSO₄, K₂CO₃, KHCO₃, and mixtures thereof. In specific instances, the at least one Salt C is Na₂SO₄. In specific instances, the at least one Salt C is provided in an amount in a range of from about 1 to about 50 equivalents with respect to the amount of the compound of Formula (I), such as in a range of from about 10 to about 30 equivalents, or an amount of about 20 equivalents with respect to the amount of the compound of Formula (I).

In a second aspect of the seventh embodiment the process further comprises treating the salt mixture with at least one Immiscible Solvent containing at least one Salt D.

In a first instance of this aspect, the at least one Immiscible Solvent is selected from the group consisting of 2-MeTHF, EtOAc, and mixtures thereof. In occurrences of this instance, the at least one Immiscible Solvent is 2-MeTHF. In specific occurrences of this instance, the at least one Immiscible Solvent is provided in an amount in a range of from about 20 volumes to about 500 volumes with respect to the amount of the compound of Formula (I), such as in an amount in a range of from about 50 volumes to about 250 volumes, or an amount of about 100 volumes.

In a second instance of this aspect, the at least one Salt D is selected from salts having a cation selected from the group consisting of

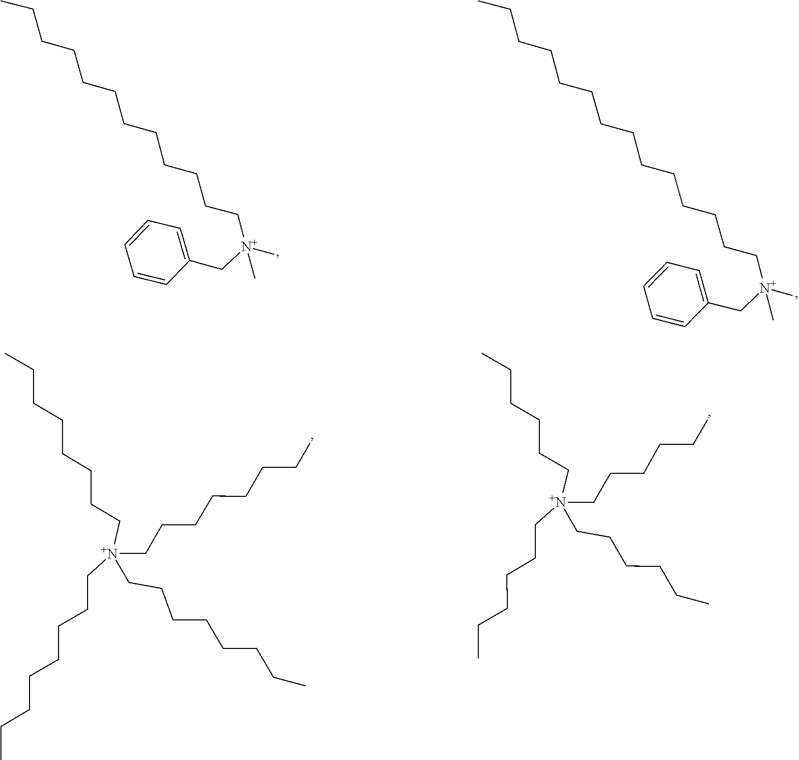

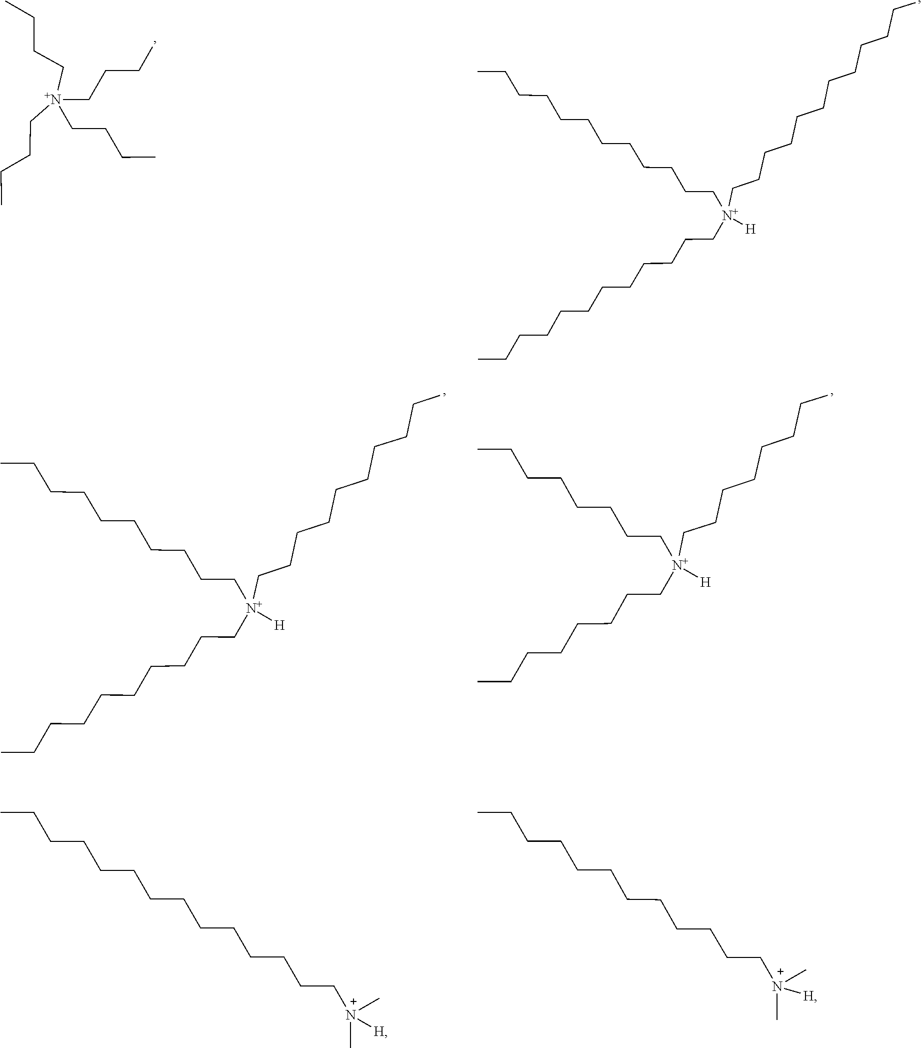

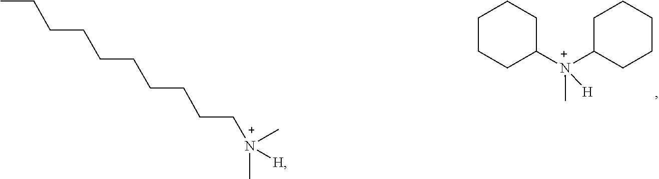

and having an anion selected from Cl⁻, Br⁻, I⁻, HSO₄⁻, SO₄²⁻, H₂PO₄⁻, HPO₄²⁻, PO₄³⁻, and mixtures thereof. In specific occurrences of this instance, the at least one Salt D is selected from salts having a cation selected from the group consisting of

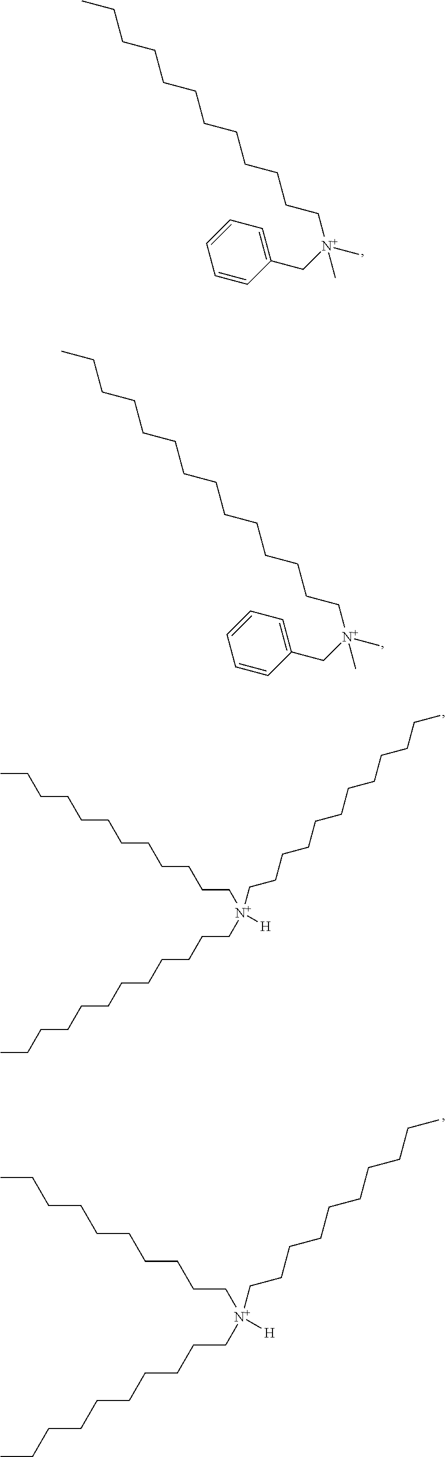

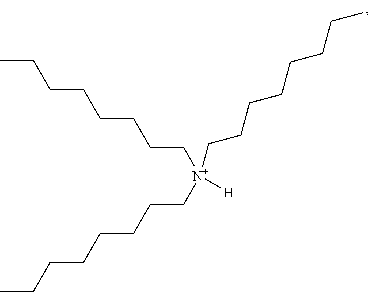 [SMILES: CCCCCCCC[NH+](CCCCCCCC)CCCCCCCC]

and having an anion selected from Cl⁻, Br⁻, I⁻, HSO₄⁻, SO₄²⁻, H₂PO₄⁻, HPO₄²⁻, PO₄³⁻, and mixtures thereof. In more specific occurrences of this instance, the at least one Salt D has having a cation that is

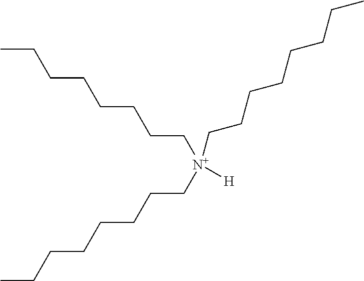 [SMILES: CCCCCCCC[NH+](CCCCCCCC)CCCCCCCC]

and having an anion selected from HSO₄⁻ and SO₄²⁻. In occurrences of this instance, the at least one Salt D is provided in an amount in a range of from about 1 to about 20 equivalents with respect to the amount of the compound of Formula (I), such as in a range of from about 3 to about 10 equivalents, or an amount of about 5 equivalents with respect to the amount of the compound of Formula (I).

In a third instance of this aspect, the at least one Immiscible Solvent further comprises at least one Co-Solvent selected from the group consisting of alcohols. In occurrences of this instance, the at least one Co-Solvent is selected from the group consisting of 1-propanol, 2-propanol, 1-butanol, 2-butanol, 1-pentanol, 1-hexanol, 1-heptanol, 1-octanol, 2-octanol, and mixtures thereof. In specific occurrences, the at least one Co-Solvent is 1-propanol. In specific occurrences of this instance, the at least one Co-Solvent is provided in an amount in a range of from about 10 volumes to about 1000 volumes with respect to the amount of the compound of Formula (I), such as in an amount in a range of from about 50 volumes to about 200 volumes, or an amount of about 93 volumes.

In a third aspect of the seventh embodiment, the process further comprises reacting the mixture with an Aqueous Salt Solution. In instances of this third aspect, the Aqueous Salt Solution contains at least one Salt E, where the at least one Salt E is selected from the group consisting of Na₂SO₄, NaHSO₄, Na₃PO₄, Na₂HPO₄, NaH₂PO₄, NaCl, K₂SO₄, KHSO₄, K₃PO₄, K₂HPO₄, KH₂PO₄, KCl, and mixtures thereof. In specific occurrences, the at least one Salt E is selected from Na₂SO₄, NaCl, and mixtures thereof. In more specific occurrences, the at least one Salt E is Na₂SO₄. In further instances of this aspect, the at least one Salt E is provided in an amount in a range of from about 0.5 to about 10 equivalents with respect to the amount of the compound of Formula (I), such as in an amount of about 1.2 equivalents with respect to the amount of the compound of Formula (I). In further instances of this third aspect, the Aqueous Salt Solution is provided in an amount in a range of from about 5 volumes to about 300 volumes with respect to the amount of the compound of Formula (I).

In a fourth aspect of the seventh embodiment, the process further comprises reacting the mixture with an Aqueous Base Solution. In instances of this fourth aspect, the Aqueous Base Solution contains at least one Base D, where the at least one Base D is selected from the group consisting of NaOH, KOH, Na₂CO₃, NaHCO₃, K₂CO₃, KHCO₃, and mixtures thereof. In specific occurrences, the at least one Base D is NaOH. In further instances of this aspect, the at least one Base D is provided in an amount in a range of from about 2 to about 40 equivalents with respect to the amount of the compound of Formula (I), such as in a range of from about 5 to about 20 equivalents, or an amount of about 10 equivalents with respect to the amount of the compound of Formula (I). In further instances of this third aspect, the Aqueous Base Solution is provided in an amount in a range of from about 5 volumes to about 300 volumes with respect to the amount of the compound of Formula (I).

In a fifth aspect of the seventh embodiment, the process further comprises crystallizing the compound of Formula (I). In instances of this embodiment, the process comprises crystallizing the compound of Formula (I) wherein each R is H. In other instances, the process comprises crystallizing the compound of Formula (I) wherein each R is Na. In further instances, the process comprises crystallizing the compound of Formula (I) wherein each R is K. In instances of this embodiment, the crystallizing comprising adding an Acid B selected from the group consisting of HCl, HBr, HI, H₃PO₄, H₂SO₄, HO₂CH, HO₂CCH₃, and mixtures thereof. In specific instances, the at least one Acid B is HCl. In more specific instances, the at least one Acid B is provided in an amount in a range of from about 0.5 to about 10 equivalents with respect to the amount of the compound of Formula (I), such as in an amount of about 1.4 equivalents.

In a sixth aspect of the seventh embodiment, the process further comprises isolating the crystallized compound of Formula (I). In instances of this aspect, the process comprises isolating the crystallized compound of Formula (I) wherein each R is H. In other instances, the process comprises isolating the crystallized compound of Formula (I) wherein each R is Na. In further instances, the process comprises isolating the crystallized compound of Formula (I) wherein each R is K. In instances of this aspect, the isolating comprises filtering and washing the crystals. In particular instances, the washing is conducted with at least one Solvent C selected from the group consisting of at least one Alcohol Solvent C, water, and mixtures thereof. In occurrences of these instances, the at least one Alcohol Solvent C is selected from the group consisting of MeOH, EtOH, 2-propanol, and mixtures thereof. In specific occurrences, the at least one Alcohol Solvent C is EtOH. In occurrences, the at least one Solvent C is a mixture of EtOH and water. In occurrences, the at least one Solvent C is a mixture of 90-95% of the at least one Alcohol Solvent C in water. In instances of this aspect, the at least one Solvent C is used in an amount in a range of from about 1 volume to about 20 volumes, with respect to the amount of the compound of Formula (I), such as about 10 volumes.

In a seventh aspect of the seventh embodiment, the process further comprises drying crystals of the compound of Formula (I). In instances of this aspect, the process comprises drying crystals of the compound of Formula (I) wherein each R is H. In other instances, the process comprises drying crystals of the compound of Formula (I) wherein each R is Na. In further instances, the process comprises drying crystals of the compound of Formula (I) wherein each R is K. In instances of this aspect, the drying is conducted under a vacuum. In instances of the aspect, the drying is conducted at a relative humidity in a range of from about 30% to about 50%, such as in a range of from about 33% to about 45%.

The second through seventh embodiments are understood to include and incorporate, as necessary and appropriate, the first through sixth embodiments in all their aspects.

In an eighth embodiment, the disclosure further provides a process for preparing a compound of Formula (I-Ia′) by reacting a compound of Formula (I-Ia) with at least one guanylate kinase type enzyme.

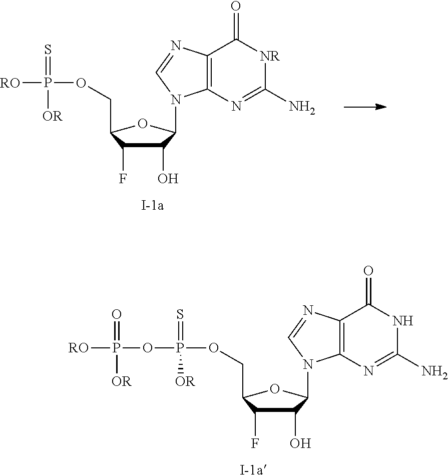

wherein each R is as defined above. In aspects of this embodiment, each R is a cation independently selected from the group consisting of H⁺, Na⁺, K⁺, Mg⁺⁺, and NH₄⁺. In specific aspects, each R is H⁺. In additional specific aspects, each R is Na⁺.

In a first aspect of the eighth embodiment, the at least one guanylate kinase type enzyme, as described above, is selected independently from the group consisting of wild-type guanylate kinase type enzymes and guanylate kinase enzymes that are the product of directed evolution from a wild-type guanylate kinase type enzyme. In specific instances, the at least one guanylate kinase type enzyme is the wild-type guanylate kinase type enzyme having the amino acid sequence that is SEQ ID NO: 14. In a first instance of this first aspect, the at least one guanylate kinase type enzyme has the amino acid sequence that is SEQ ID NO: 15. In a second instance of this first aspect, the at least one guanylate kinase type enzyme has the amino acid sequence that is SEQ ID NO: 16. In a third instance of this first aspect, the at least one guanylate kinase type enzyme has the amino acid sequence that is SEQ ID NO: 17. In a fourth instance of this first aspect, the at least one guanylate kinase type enzyme has the amino acid sequence that is SEQ ID NO: 18. In a fifth instance of this first aspect, the at least one guanylate kinase type enzyme has the amino acid sequence that is SEQ ID NO: 19.

In even more specific instances of this aspect, the at least one guanylate kinase type enzyme is selected from wild-type guanylate kinase type enzyme, which has the amino acid sequence as set forth above in SEQ ID NO: 14, and guanylate kinase type enzymes that are the product of directed evolution from a wild-type guanylate kinase type enzyme and that have the amino acid sequences as set forth above in SEQ ID NO: 15, SEQ ID NO: 16, SEQ ID NO: 17, SEQ ID NO: 18, and SEQ ID NO: 19.

In a sixth instance of this first aspect, the at least one guanylate kinase type enzyme is selected from the group consisting of guanylate kinase type enzymes and immobilized guanylate kinase type enzymes, as described above with respect to the second embodiment.

In a second aspect of the eighth embodiment, the reacting is conducted in the presence of at least one Co-Factor A. In instances of this aspect, the at least one Co-Factor A is 2′F-thio-ATP or natural ATP. In specific instances of this aspect, the at least one Co-Factor A is provided in an amount in a range of from about 0.0005 to 2.0 equivalents with respect to the amount of the compound of Formula (I-1a).

In a third aspect of the eighth embodiment, the reacting is conducted in the presence of at least one Metal Co-Factor B. In instances of this aspect, the at least one Metal Co-Factor B is selected from the group consisting of MgSO₄, MgCl₂, MnCl₂, and Mg(OH)₂, hydrates thereof, and mixtures thereof. In specific instances, the at least one Metal Co-Factor B is MgCl₂. In specific instances of this aspect, the at least one Metal Co-Factor B is provided in an amount in a range of from about 0.1 to 5.0 equivalents with respect to the amount of the compound of Formula (I-1a).

In a fourth aspect of the eighth embodiment, the reacting is conducted in the presence of at least one Inorganic Salt D selected from the group consisting of KCl, KBr, and NaCl, and mixtures thereof. In specific instances of this aspect, the at least one Inorganic Salt D is KCl. In specific instances of this aspect, the at least one Inorganic Salt D is provided in an amount in a range of from about 0.1 to 10.0 equivalents with respect to the amount of the compound of Formula (I-1a).

In a fifth aspect of the eighth embodiment, the reacting is conducted in the presence of at least one Salt A selected from the group consisting of AcP—Li/Li, AcP—Na/Na, AcP—K/K, AcP—Li/K, AcP—NH₄/NH₄, and mixtures thereof. In specific instances of this aspect, the at least one Salt A is AcP—Li/Li. In specific instances of this aspect, the at least one Salt A is provided in an amount in a range of from about 0.5 to 7.0 equivalents with respect to the amount of the compound of Formula (I-1a).

In a sixth aspect of the eighth embodiment, the reacting is conducted in the presence of at least one Solvent B.

In a first instance of the sixth aspect, the at least one Solvent B is water. In specific occurrences of this instance, water is provided in an amount in a range of from about 15 to 50 volumes with respect to the amount of the compound of Formula (I-1a).

In a second instance of the sixth aspect, the at least one Solvent B is selected from water in combination with at least one organic solvent. In instances of this aspect, the at least one Solvent B is selected from the group consisting of EtOH, MeOH, iPrOH, MeCN, DMSO, TGDE, EtOAc, acetone, and tBuOH, and mixtures thereof. In a specific instance of this aspect, the at least one Solvent B is water in combination with EtOH.

In a seventh aspect of the eighth embodiment, the reacting is conducted in a temperature range of from about −10° C. to about 35° C. In instances of this aspect, the reacting is conducted in a temperature range of from about 0° C. to about 25° C.

In an eighth aspect of the eighth embodiment, the reacting is conducted in the presence of Base C, which is selected from the group consisting of KOH, NaOH, and mixtures thereof. In specific instances, Base C is KOH. In other specific instances, Base C is NaOH. In instances of this embodiment, Base C is included in an amount sufficient to control pH in a range of from about 5.5 to about 8.5. In instances of this embodiment, Base C is included in an amount sufficient to control pH in a range of from about 6.4 to about 7.0 at a temperature of about 25° C.

In a ninth embodiment, the disclosure further provides a process for preparing a compound of Formula (I-2a′) by reacting a compound of Formula (I-2a) with at least one adenylate kinase enzyme.

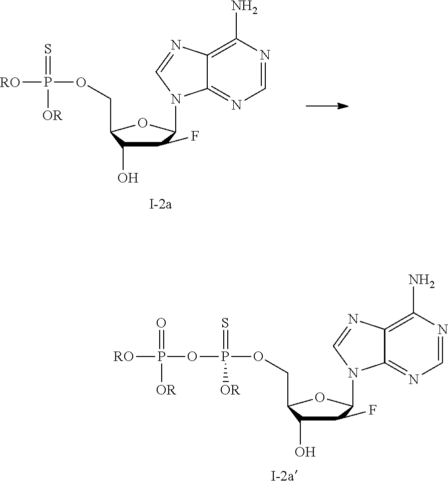

wherein each R is as defined above. In aspects of this embodiment, each R is a cation independently selected from the group consisting of H⁺, Na⁺, K⁺, Mg⁺⁺, and NH₄⁺.

In a first aspect of the ninth embodiment, the at least one adenylate kinase enzyme is selected independently from the group consisting of wild-type adenylate kinase type enzymes and adenylate kinase enzymes that are the product of directed evolution from a wild-type adenylate kinase type enzyme. In specific instances, the at least one adenylate kinase type enzyme is the wild-type adenylate kinase type enzyme having the amino acid sequence that is SEQ ID NO: 23. In a first instance of this ninth aspect, the at least one adenylate kinase type enzyme has the amino acid sequence that is SEQ ID NO: 24. In a second instance of this ninth aspect, the at least one adenylate kinase type enzyme has the amino acid sequence that is SEQ ID NO: 25. In a third instance of this ninth aspect, the at least one adenylate kinase type enzyme has the amino acid sequence that is SEQ ID NO: 26. In a fourth instance of this ninth aspect, the at least one adenylate kinase type enzyme has the amino acid sequence that is SEQ ID NO: 27.

In even more specific instances of this first aspect, the at least one adenylate kinase type enzyme is selected from wild-type adenylate kinase type enzyme, which has the amino acid sequence as set forth above in SEQ ID NO: 23, and adenylate kinase type enzymes that are the product of directed evolution from a wild-type adenylate kinase type enzyme and that have the amino acid sequences as set forth above in SEQ ID NO: 24, SEQ ID NO: 25, SEQ ID NO: 26, and SEQ ID NO: 27.

In instances of this first aspect, the at least one adenylate kinase type enzyme is selected from the group consisting of adenylate kinase type enzymes and immobilized adenylate kinase type enzymes, with respect to the third embodiment.

In a second aspect of the ninth embodiment, the reacting is conducted in the presence of a Co-Factor B. In instances of this aspect, the Co-Factor B is 2′F-thio-ATP or natural ATP. In specific instances of this aspect, the Co-Factor B is provided in an amount in a range of from about 0.0001 to 2 equivalents with respect to the amount of the compound of Formula (I-2a).

In a third aspect of the ninth embodiment, the reacting is conducted in the presence of water. In specific instances, water is provided in an amount in a range of from about 20 to 50 volumes with respect to the amount of the compound of Formula (I-2a).

In a fourth aspect of the ninth embodiment, the reacting is conducted in the presence of a Metal Salt A. In instances of this aspect, the Metal Salt A is selected from the group consisting of divalent metal salts, hydrates thereof, and mixtures thereof. In specific instances, the at least one Metal Salt A is MgCl₂·(H₂O)₆. In specific instances of this aspect, the Metal Salt A is provided in an amount in a range of from about 0.125 to 1.5 equivalents with respect to the amount of the compound of Formula (I-2a).

In a fifth aspect of the ninth embodiment, the reacting is conducted in a temperature range of from about 5° C. to about 30° C. In instances of this aspect, the reacting is conducted in a temperature range of from about 10° C. to about 25° C. In specific instances, the reacting is conducted at a temperature of about 10° C. In instances of this aspect, the reacting is conducted over a time period in a range of about 10 h to about 100 h, such as over a time period in a range of about 20 h to about 80 h, over a time period in a range of about 30 h to about 50 h, over a time period of about 40 h.

In a tenth embodiment, the disclosure further provides compounds selected from the group consisting of

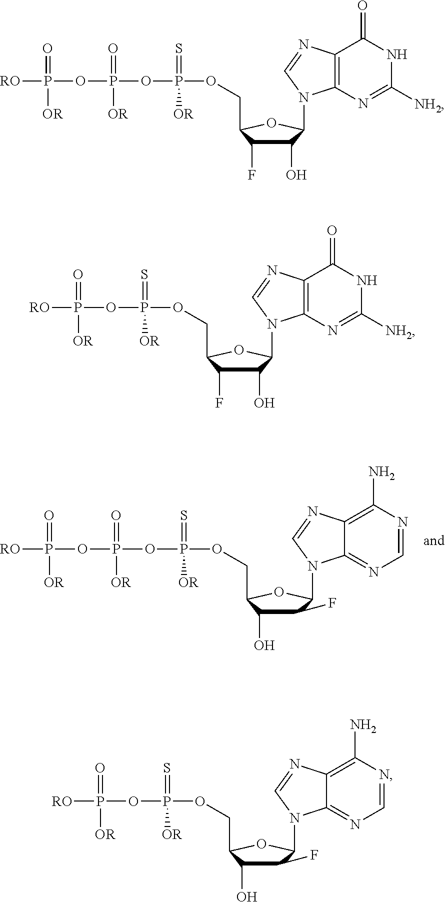

wherein each R is as defined above. In aspects of this embodiment, each R is a cation independently selected from the group consisting of H⁺, Na⁺, K⁺, Mg⁺⁺ and NH₄⁺. In more specific aspects, each R is Na⁺.

### EXAMPLES

### Example 1: Synthesis of (trisodium O-{[(2R,3S,4S,5R)-5-(2-amino-6-oxido-9H-purin-9-yl)-3-fluoro-4-hydroxyoxolan-2yl]methyl} phosphorothioate hydrate (1:6))

### Step 1: Synthesis of (2R,3R,4R,5R)-2-(2-amino-6-oxo-1,6-dihydro-9H-purin-9-yl)-5-(((tert-butyldimethylsilyl)oxy)methyl)tetrahydrofuran-3,4-diyl bis(4-methylbenzenesulfonate)

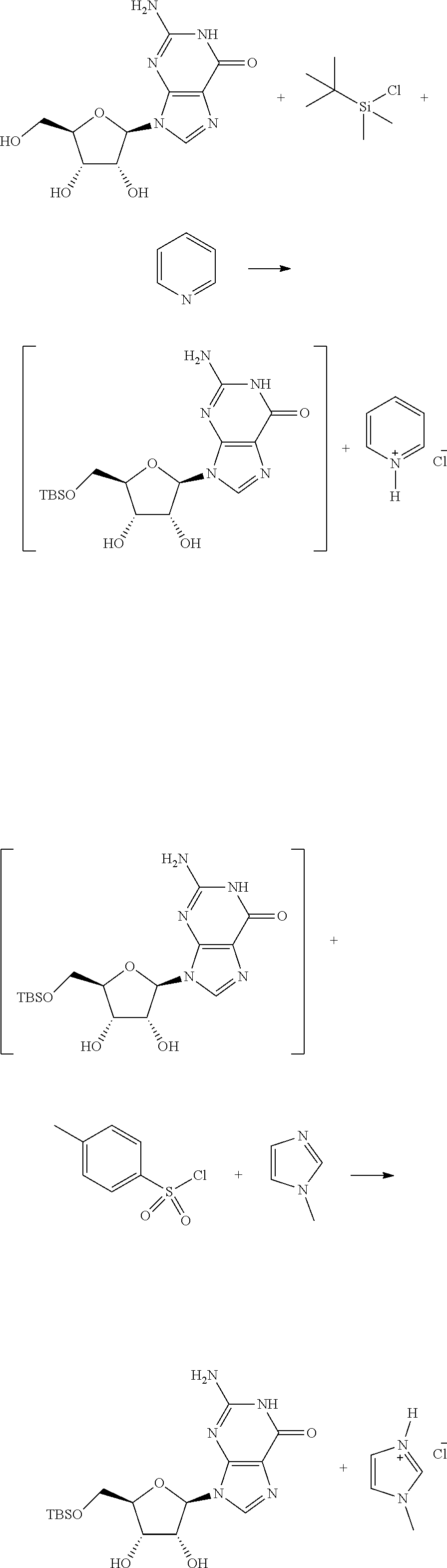

NMP (3.5 vol.) was added into a reaction vessel, and the temperature was adjusted to 48° C. to 52° C. Guanosine (800 g, 2824 mmol) was added. The reaction mixture was stirred for 30 min. to 1 h, and the temperature was adjusted to 8° C. to 12° C. TBS-Cl (575 g, 3815 mmol) (dissolved in 2 vol. NMP) was added into the reaction mixture (total NMP 5.5 vol.), and the reaction mixture was maintained at 8° C. to 12° C. Py (670 g, 8470 mmol) was added to the reaction mixture, which was maintained at 8° C. to 12° C. and stirred for 3 to 4 h. The temperature was adjusted to −20° C. to −10° C., and the reaction mixture was stirred for 8 to 15 h, after which the temperature was adjusted to −5° C. to 5° C.

NMI (2319 g, 28240 mmol) was added to the reaction mixture, which was kept at −5° C. to 5° C. To the reaction mixture, 2.1 eq. Ts-Cl (1131 g dissolved in 3 vol. 2-Me-THF) was added, and the reaction mixture was stirred at −5° C. to 5° C. for 4 to 8 h. Then, 0.7 eq. Ts-Cl (377 g, dissolved in 1 vol. 2-Me-THF) was added. The reaction mixture was stirred for 12 to 14 h at −5° C. to 5° C. Ts-Cl (0.16 eq, 86 g dissolved in 160 mL 2-Me-THF) was added to the reaction mixture, which was stirred for 3 to 5 h. MeOH (5.5 vol.) was added to the reaction mixture at 15° C. to 25° C., followed by water (8 vol.). The reaction mixture was stirred at this temperature for 12 to 15 h. The reaction mixture was then filtered and rinsed with 2 vol. MeOH/water (1:3). The reaction product was dried under 45° C. for 70 h in two parts.

### Step 2: Synthesis of (2R,3R,4R,5R)-2-(((tert-butyldimethylsilyl)oxy)methyl)-5-(2-isobutyramido-6-oxo-1,6-dihydro-9H-purin-9-yl)tetrahydrofuran-3,4-diyl bis(4-methylbenzenesulfonate)

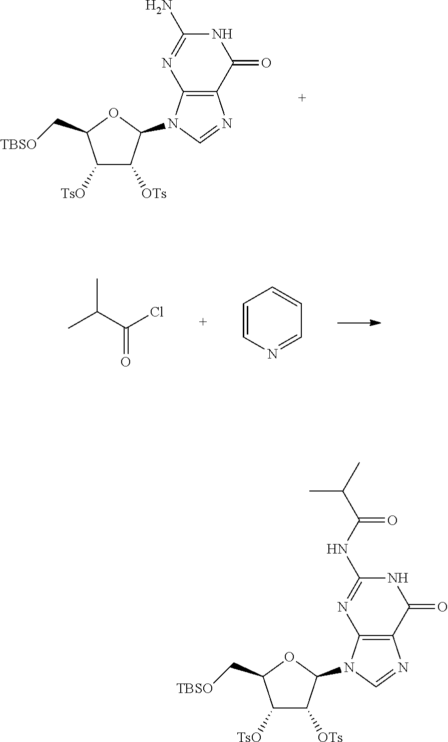

The bis-tosylate (1096.00 g (1185.50 g×92.45%)) was charged into a reaction vessel. MeCN (3.3 L, 3 vol.) and Py (510.52 g, 4.2 eq.) then were charged into the reaction vessel. The reaction mixture was cooled to −15° C. to −5° C. (slurry). Isobutyryl chloride (397.51 g, 2.4 eq.) was added by dropwise to the reaction mixture under −5° C. (slurry). The reaction mixture was stirred at −15° C. to −5° C. for 18 h. Isopropyl acetate (6 L, 5 vol.) was charged into the reaction mixture, and 15% K₂CO₃ liquor (6 kg) was added by dropwise into the reaction mixture under −5° C. The reaction mixture was stirred at −15° C. to −5° C. for 30 min. The reaction mixture was then warmed to 20° C. to 30° C. and stirred for 10 to 30 min.

The reaction mixture was separated, and the aqueous layer was removed. The organic layer was concentrated to 3-4 vol. at 30° C. IPAc (6 L, 5-6 vol.) was charged into the concentrated organic layer, which was then stirred at 25° C. to 30° C. for 30 min. The organic layer was then further concentrated until it reached 5-6 vol. under 30° C. An additional IPAc (2 L, 2-3 vol.) was charged into the concentrated organic layer, and it was stirred at 25° C. to 30° C. for 30 min. The reaction mixture was cooled to 15° C. to 25° C. 3 L (3 vol.) n-heptane was added drop-wise at 15° C. to 25° C. for 6 h, then the reaction mixture was stirred for 10 h 25° C. to 30° C. 3 L n-heptane was added drop-wise at 15° C. to 25° C. for 6 h, and the reaction mixture was stirred at 25° C. to 30° C. for 10 h. The suspension was filtered, and the filter cake was washed with 2 L mixture solution (IPAc/n-heptane=1 L/1 L) to give the product, which was dried in oven under 35° C. by reduce for 24 h.

¹H NMR (500 MHz, DMSO-d6) δ 11.97 (s, 1H), 11.50 (s, 1H), 7.89 (d, J=8.3 Hz, 2H), 7.86 (s, 1H), 7.55 (d, J=8.2 Hz, 2H), 7.38 (d, J=8.2 Hz, 2H), 7.07 (d, J=8.2 Hz, 2H), 6.00 (d, J=7.9 Hz, 1H), 5.58 (dd, J=7.8, 5.4 Hz, 1H), 5.05 (d, J=5.3 Hz, 1H), 4.27 (t, J=4.5 Hz, 1H), 3.85 (dd, J=12.2, 4.1 Hz, 1H), 3.70-3.66 (m, 1H), 2.76 (septet, J=6.8 Hz, 1H), 2.46 (s, 3H), 2.26 (s, 3H), 1.18 (t, J=7.2 Hz, 6H), 0.87 (s, 9H), 0.06 (s, 3H), 0.04 (s, 3H).

¹³C NMR (125 MHz, DMSO-d6) δ 180.4, 154.8, 148.5, 148.3, 146.3, 146.0, 137.1, 132.6, 131.6, 131.0, 130.0, 128.3, 127.3, 120.59, 83.9, 83.4, 78.5, 76.8, 62.7, 35.4, 31.7, 28.8, 26.2, 21.7, 21.4, 19.4, 19.2, 18.4, 14.4, −5.1, −5.1.

### Step 3: Ketone Synthesis

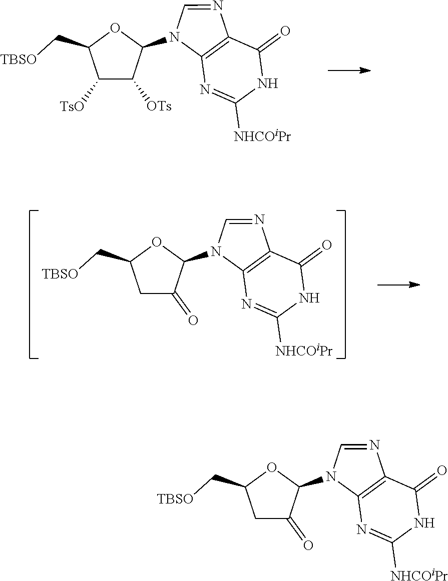

A first reaction vessel was charged with i-PrOH (3.88 L, 50.30 mol) and THF (9.75 L, 1.5 vol) at RT and placed under N₂ before being cooled to −15° C. n-Butyllithium (19.18 L, 47.90 mol, 2.5M in hexanes) was then added slowly, maintaining internal temperature below 25° C. A second vessel was placed under N₂ and charged with the bis-tosylate (6.5 kg, 7.99 mol, 96 wt %) and CPME (26 L, 4 vol.) before being cooled to −5° C. The solution of lithium isopropoxide from the first reaction vessel was then vacuum transferred to the slurry in the second reaction vessel, and the mixture was warmed to 0° C. and aged for about 18 h. The slurry was cooled to −10° C., and AcOH (2.74 L, 47.90 mol) was added slowly, maintaining internal temperature below 5° C. To this mixture was added DI water (32.5 L, 5 vol.), the phases were separated, and the aqueous phase was removed from reactor. The organic layer was cooled to −10° C., and TFA (3.08 L, 40.00 mol) was added slowly, maintaining the internal temperature below 5° C., followed by trioctylamine (6.99 L, 15.98 mol). The mixture was warmed to 0° C. and aged for about 16 h. The slurry was cooled to −10° C., and trioctylamine (10.48 L, 23.97 mol) was added slowly. The resulting homogenous solution was warmed to 25° C. and seeded with 1 wt % of the ketone (26.7 g, 0.0799 mol) and aged for 18 h. The slurry was filtered, and the cake was completely deliquored. The cake was then slurry washed twice with CPME (3.25 L, 0.5 vol.) and then dried under vacuum with N₂ sweep.

¹H NMR (500 MHz, DMSO-d6) δ 12.12 (s, 1H), 11.47 (s, 1H), 8.11 (s, 1H), 5.99 (s, 1H), 5.05 (t, J=5.6 Hz, 1H), 4.54 (app. dq, J=8.6, 5.4, 4.4 Hz, 1H), 3.71-3.60 (m, 2H), 2.97 (dd, J=18.5, 8.4 Hz, 1H), 2.85-2.75 (m, 2H), 1.14 (app. d, J=6.8 Hz, 6H).

¹³C NMR (125 MHz, DMSO-d6) δ 209.0, 180.6, 155.2, 149.0, 148.6, 139.4, 120.4, 81.7, 77.1, 63.8, 38.0, 35.3, 19.4, 19.3.

### Step 4: Ketone Fluorination

NFSI (1.964 kg in 5.5 L THF) was charged into a first reaction vessel. The ketone (1.832 kg) was then charged into a separate reaction vessel, followed by THF (5.5 L), H₂O (0.932 L) and L-leucine amide hydrochloride (259 g). The reaction mixture in the second reaction vessel was agitated at 70 rpm at RT. After 40 min., the reaction temperature was 20° C., and 1.5 L NFSI solution (˜20%) from the first reaction vessel was added to the second reaction vessel, followed immediately by 1.371 kg (NH₄)₂HPO₄. The agitation was set 80 rpm. After 20 min., the remainder of the NFSI from the first reaction vessel was charged into the second reaction vessel over 90 min., and the reaction mixture was left for 2 h at 27° C. THF (200 mL) was added to rinse the bottle, and the mixture became homogenous as the temperature increased to 27.9° C. The agitation was then set to 92 rpm. The reaction mixture was then aged for 42 h.

While the temperature was maintained at 27° C., H₂O (10 vol, 18.32 L) was charged into the reaction mixture over 40 min. The reaction mixture was concentrated by distillation, removing THF in batches. Once the distillation was completed, the slurry was allowed to de-supersaturate at 22° C. overnight.

The reaction mixture was set to agitate at 47 rpm. The reaction mixture was filtered under vacuum. The wet cake was then washed with 1 IL H₂O, followed by MeCN (2×5.5 L). The wet cake was then dried under N₂ sweep for a period of two and a half days.

¹H NMR (500 MHz, DMSO-d₆: D₂O (5:1)) δ 12.08 (s, 1H), 11.69 (s, 1H), 8.02 (s, 1H), 7.00 (br s, 2H), 5.85 (d, J=1.9 Hz, 1H), 5.15 (br s, 1H), 4.83 (dd, J=53.6, 2.7 Hz, 1H), 4.06 (dddd, J=26.2, 8.3, 2.9, 2.8 Hz, 1H), 3.70-3.63 (m, 2H), 2.77 (sept, J=6.8 Hz, 1H), 1.12 (d, J=6.7 Hz, 6H).

¹³C NMR (126 MHz, DMSO-d₆: D₂O (5:1)) δ 180.7, 155.6, 149.6, 148.3, 139.7, 119.5, 97.2 (d, J=17.8 Hz), 93.4 (d, J=188.7 Hz), 86.1, 81.8 (d, J=23.2 Hz), 60.8 (d, J=7.6 Hz), 35.3, 19.2, 19.1.

¹⁹F NMR (470 MHz, DMSO-d₆: D₂O (5:1)) 6-189.1 (dd, J=53.6, 26.1 Hz).

### Step 5: Chemical Ketone Reduction to 3′-FG

A first reaction vessel was charged with HOAc (2.8 L, 2.0 vol) and MeCN (4.2 L, 3.0 vol) followed by STAB (2.30 kg, 3.0 eq). The walls of the first reaction vessel were rinsed with MeCN (2.8 L, 2.0 V). The resulting solution had an internal temperature of 14° C. and was heated to 22° C. over 1 h. The resulting solution was then stirred for 3 h at RT.

A second reaction vessel was charged with HOAc (4.2 L, 3 vol.) and MeCN (6.3 L, 4.5 vol.) followed by the fluorinated ketone (1.40 kg, 3.0 eq.). The vessel walls were rinsed with MeCN (2.1 L, 1.5 vol.). The resulting slurry was heated to 35° C. over 40 min. The solution of STAB from the first reaction vessel was added to the slurry over approximately 2 h. The resulting slurry was stirred for 2 h at 35° C. to 40° C., before the slurry was cooled to 25° C. over 30 min. MeOH (2.8 L, 2 vol.) was added over 1 h, and the resulting solution was allowed to stir for 13.5 h at RT.

The reaction vessel was placed under vacuum for distillation, and the temperature was set to 50° C., with distillation starting when the internal temperature reached to 35° C. Distillation was continued until total ˜4 vol. (5.6 L) of the reaction mixture remained. DI water (2.8 L) was added over 6 min when internal temperature reached 55° C. The walls were washed with water. (NH₄)₂SO₄ (2.8 L, 2 vol.) was added over 20 min to the washed reaction solution.

The reaction mixture was aged for 40 min. Following aging, (NH₄)₂SO₄ (22.4 L, 16 vol.) was added over 4 h, and the slurry was aged again for 2 h at 55° C. The reaction mixture was cooled to RT over 5 h, and then aged at RT for 5.5 h.

After aging, the reaction mixture was filtered, and the filter cake was washed with 4.3 L of H₂O:MeOH (3:1) twice. The cake was then dried under N₂ sweep and vacuum.

¹H NMR (500M Hz, DMSO-d₆) δ 11.68 (s, 2H), 8.27 (s, 1H), 5.96 (d, J=5.4 Hz, 1H), 5.83 (d, J=8.1 Hz, 1H), 5.22 (t, J=5.4 Hz, 1H), 5.07 (dd, J=54.3, 4.1 Hz, 1H), 4.77 (dddd, 27.3, 8.1, 4.1, 4.1 Hz, 1H), 4.25 (dddd, J=27.2, 8.1, 4.1 Hz, 4.1 Hz, 1H), 3.61 (m, 2H), 2.75 (sept, J=6.8 Hz, 1H), 1.12 (d, J=6.8 Hz, 6H).

¹³C NMR (125 MHz, DMSO-d₆) δ 180.2, 154.8, 149.3, 148.4, 137.4, 120.1, 92.8 (d, J=183 Hz), 85.0, 83.6 (d, J=21.1 Hz), 83.5, 72.6 (d, J=16.1 Hz), 60.6 (d, J=11.2 Hz), 34.8, 18.8, 18.8.

¹⁹F NMR (500 MHz, DMSO-d₆) δ −197.5.

### Step 6: Biocatalytic Ketone Reduction to 3′-FG (Alternative to Step 5)

10 uL of a ketoreductase enzyme that has the amino acid sequence that is SEQ ID NO: 28, as set forth below, was inoculated into 5 mL of Luria-Bretani Broth (culture media for cells), supplemented with 1% glucose and 50 ug/ml of Kanamycin antibiotic and grown overnight for 20-24 h at 30° C., 250 rpm, in a shaking incubator.

(SEQ ID NO: 28)
MHHHHHPATIVVTGGTKGIGRAIVEKFAKEGFTVLTCARTKGDNFPENVH
FFKADLSKKVEVLAFADFIKQTVNQVDILVNNTGWFLPGEINNEAEGTLE
AMIETNLYSAYYLTRALVGDMITKKEGHIFNICSYASIVPYTSGGSYCIS
KTAQLGMSKVLREELKPHHVRVTSILPGAVLNDSWAKVELPAELFIAPED
IAQIVWTAHCLPSTTVLEEILIRPQTGDL

5 mL of the overnight culture was used to subculture 250 mL of Terrific Broth growth media (commercially available from ThermoFisher Scientific as Catalog #A1374301) in a 1 L flask. The subculture was allowed to grow at 30° C. for 3 h at 250 rpm, in a shaking incubator. When the OD measures between 0.4 and 0.6, the IPTG was introduced to an IPTG final concentration of 1 Mm (1 mM). The subculture was allowed to grow overnight, for 18-20 h.

After the growth period, the culture was transferred to a centrifuge bottle and centrifuged for 20 min. at 4000 rpm at 4° C. Following centrifuge, the supernatant was discarded. The cell pellets were resuspended in 50 mM sodium phosphate buffer (pH=7).

The cells from the resuspended cell pellets were lysed using a microfluidizer, and the cell lysate was collected and centrifuged for 60 min. at 10000 rpm at 4° C. The supernatant was transferred to a petri dish and frozen at −80° C. for a minimum of 2 h. Samples were optionally lyophilized using a standard automated protocol.

20 mg of a commercially available ketoreductase enzyme (KRED-P1 B10, commercially available from CODEXIS, Inc.) added to a reaction vessel, along with NADPH (20 mg), a ketoreductase enzyme that can be represented by SEQ ID NO: 28, as set forth above (250 mg, harvested from the subculture), and fluoroketone (250 mg, step 4 above). 10 mL of phosphate buffer (0.1M, ph=6.0) and 1 mL IPA were added to the reaction vessel. The temperature was set at 30° C., and the reaction mixture was stirred at 350 rpm. After 20 h, the mixture was cooled to 15° C. NaCl (2 g) was added to the reaction vessel, and the reaction mixture was allowed to de-supersaturate overnight. The solids were filtered and washed with 2.5 mL (10 vol) of water. The wet cake was placed into a 50° C. vacuum oven to dry overnight.

### Step 7: Thiophosphorylation

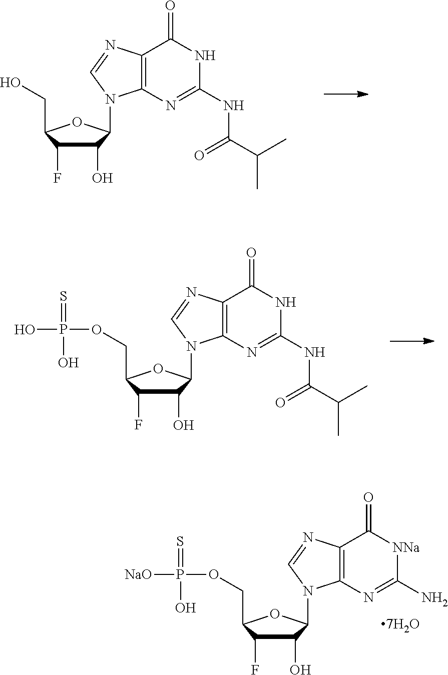

A reaction vessel at 22° C. was charged with THF (16 L). Quinine (234 g, 0.72 mol, 0.25 equiv) was charged into the reaction vessel, followed by 2,6-lutidine (463 g, 4.32 mol, 1.5 equiv), and 3′-FG (1100 g, 2.88 mol, 1.0 equiv, step 6 above). THF (6 L) was used to rinse the sides of the reaction vessel, and the temperature was set to 0° C. PSCl₃ (658 g, 3.89 mol, 1.35 equiv) was charged, maintaining the temperature below 2° C. The reaction mixture was stirred at 80 rpm for approximately 40 h at 0° C. The reaction progress was monitored by UPLC analysis; once 96% conversion had been obtained, the reaction temperature was adjusted to −10° C. H₂O (2.2 L) was added dropwise, maintaining the temperature below 0° C. After the addition, the temperature was adjusted to 25° C., and the reaction mixture was held at this temperature for 1 h.

The THF was removed in vacuo. After THF removal (at least 17 vol.), the vacuum was broken, and the temperature was set to 25° C. MeOH (11 L) was charged into the reaction vessel, and the temperature was adjusted to −10° C. Aqueous NaOH (50 wt %) was diluted with H₂O (1.1 L) and charged into the reaction vessel, maintaining the temperature below 25° C.

The temperature was then adjusted to 45° C., and, after 90 min, the reaction mixture was seeded with 3′-F-thio-GMP (1 wt %, 11 g). The mixture was held at 45° C. for 5 h, then cooled to 20° C. over 5 h, and held at 20° C. THF (1.8 L, 1.6 V) added over 45 min at 20° C., and the mixture was agitated for 3 h. The mixture was then filtered, and the wet cake was washed with 10:4:2 MeOH:THF:H₂O (10 L). The cake was then washed with THF (10 L), and the cake was dried under vacuum under a sweep of humidified N₂.

¹H NMR (500 MHz, D₂O): δ 8.17 (s, 1H), 5.94 (d, J=8.4 Hz, 1H), 5.30 (dd, J=54.2, 4.3 Hz, 1H), 4.93 (ddd, J=25.9, 8.4, 4.3 Hz, 1H), 4.63-4.54 (m, 1H), 4.08-4.00 (m, 1H), 3.97-3.89 (m, 1H).

¹³C NMR (126 MHz, D₂O): 167.7, 161.0, 152.15, 135.93, 117.41, 92.53-93.96 (d), 84.68, 82.95 (m), 73.25 (m), 63.50 (m).

¹⁹F NMR (470 MHz, D₂O) 6-197.9.

³¹P NMR (203 MHz, D₂O) δ 43.31.

### Example 2: 3′F-thio-GTP from 3′F-thio-GMP intermediate

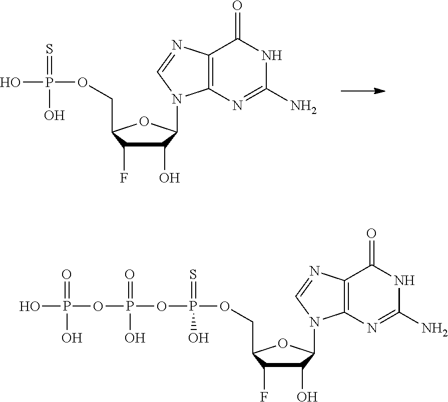

To a 200 mL reactor was charged 3′F-thio-GMP (2.5 g, 73.5 wt %, 1.0 eq), followed by addition of AcP—Li/Li (1.73 g, 94.9 wt %, 2.5 eq). Next, 2′F-thio-ATP (140 mg, 81 wt %, 0.05 eq) was charged at 25° C., followed by addition of water (50 mL) and EtOH (12.5 mL), respectively. To the resulting mixture was added MgCl₂ (4.33 mL, 1M, 1.0 eq) and KCl (2.16 mL, 3M, 1.5 eq). Then, pH was adjusted to 6.4-6.6 using HCl (9M) solution while the solution was agitated 25° C. Next, a kinase enzyme that has the amino acid sequence that is SEQ ID NO: 14 (26.5 mg) and a kinase enzyme that has the amino acid sequence that is SEQ ID NO: 25 (26.5 mg) were charged to the reactor. The mixture was stirred at 25° C. until completion (6-8 h) using overhead stirrer. After completion (˜97% conversion), HOAc was added (1.8 mL) to adjust the pH to 4.3-5.0, followed by slow addition of MeCN (-250 mL) to precipitate the product. At the end, the slurry was filtered, and the wet cake was washed with the same solvent ratio as mother liquor (ML) to afford the product, which was dried under vacuum to give 3′F-thio-GTP.

The product was redissolved in water (40 mL), and the pH was adjusted with KOH to pH 7.0. To the solution, KCl (800 μl, 3M, 0.1 eq) was added, followed by HOAc (1.6 mL). MeCN (57 mL) was added slowly to precipitate 3′F-thio-GTP. The slurry was filtered, and the wet cake was washed with the same solvent ratio as mother liquor (ML) and dried under vacuum to give 3′F-thio-GTP.

### Example 3: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate

### Step 1: Synthesis of Trimethyl(((2R,3S)-3-((trimethylsilyl)oxy)-2,3-dihydrofuran-2-yl)methoxy) silane from Thymidine

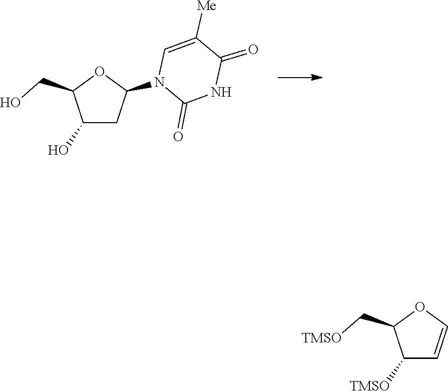

A 100 L vessel was charged with toluene (14.5 L), thymidine (4825 g, 20 mol), 2,6-lutidine (1081 g, 0.400 mol), PTPI (90 g, 0.200 mol) and heptane (33.8 L). The mixture was heated to 90° C. To this, BSA (17.4 kg, 85.6 mol) was added over 30 min. The mixture was heated to 100° C. and stirred at 100-107° C. for 3 h. After cooling to room temperature, the reaction mixture was transferred to another 100 L reactor containing i-PrOH (12.3 L, 161 mol) slowly (204 ml/min). Toluene (1 L) was used to rinse and transfer any remaining material in the first reactor. The resulting slurry was stirred at 35° C. for 2 h, then cooled to 10° C. and aged at that temperature overnight. The resulting slurry was filtered, and the filter cake was washed with heptane (20.0 L). The combined filtrates were passed through a plug of basic alumina and transferred to a 100 L vessel. The resulting solution was concentrated under vacuum to the total volume of 24 L, which was used in the subsequent reaction without further purification.

¹H NMR (400 MHz, CD₂Cl₂); δ 6.50 (dd, J=2.7, 1.1 Hz, 1H), 5.06 (t, J=2.7 Hz, 1H), 4.84 (td, J=2.7, 1.0 Hz, 1H), 4.28 (ddd, J=6.7, 6.1, 2.7 Hz, 1H), 3.67 (dd, J=10.6, 6.1 Hz, 1H), 3.47 (dd, J=10.6, 6.7 Hz, 1H), 0.17 (s, 9H), 0.16 (s, 9H).

### Step 1, alternate route: Synthesis of Trimethyl(((2R,3S)-3-((trimethylsilyl)oxy)-2,3-dihydrofuran-2-yl)methoxy) silane from 2′-Deoxyuridine

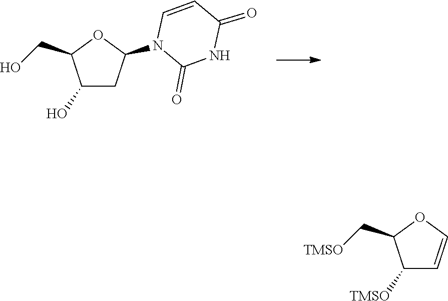

In an 8 mL vial, dry 2′-deoxyuridine (1 mmol), PTPI (0.01 eq, 5 mg), 2,6-lutidine (0.5 eq, 58 μL), 1 mL heptane, 1 mL toluene, and 3.5 eq. of BSA was added under nitrogen atmosphere. The reaction was stirred at 100° C. for 3 h. Reaction progress was monitored via HPLC by the presence of starting material.

### Step 1, alternate route: Synthesis of 2-tert-butyl(((2R,3S)-3-((tert-butyldimethylsilyl)oxy)-2,3-dihydrofuran-2-yl)methoxy)dimethylsilane (2-TBS)

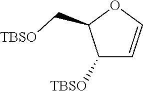 [SMILES: CC(C)(C)[Si](C)(C)OC[C@H]1OC=C[C@@H]1O[Si](C)(C)C(C)(C)C]

In a 2 L flask was charged ammonium sulfate (5.38 g, 40.7 mmol), bis(tert-butyldimethylsilyl)thymidine (100 g, 203 mmol), and 2,6-di-tert-butyl-4-methylphenol (0.045 g, 0.203 mmol). HMDS (141 mL, 671 mmol) and heptane (1000 mL) were subsequently added, and the reaction mixture was heated to reflux (140° C. external bath) under nitrogen atmosphere. After 34 h, the reaction mixture was cooled to ambient temperature. 2,4,6-trimethylpyridine (13.55 mL, 102 mmol) was added followed by ethanol (35.6 mL, 610 mmol) via syringe pump over 2 h. The resulting slurry was then filtered, and the cake was washed with CPME (4×150 mL). The filtrate was concentrated to provide 2-TBS (57.14 g, 166 mmol,) by quantitative NMR analysis.

¹H NMR (500 MHz, CDCl₃) δ 6.47 (dd, J=2.6, 0.8 Hz, 1H), 5.01 (t, J=2.6 Hz, 1H), 4.87 (td, J=2.6, 0.8 Hz, 1H), 4.29 (td, J=6.0, 2.8 Hz, 1H), 3.69 (dd, J=10.7, 5.7 Hz, 1H), 3.51 (dd, J=10.7, 6.3 Hz, 1H), 0.90 (s, 9H), 0.89 (s, 9H), 0.09 (s, 3H), 0.09 (s, 3H), 0.07 (s, 3H), 0.06 (s, 3H).

¹³C NMR (126 MHz, CDCl₃) δ 149.1, 103.6, 89.1, 76.1, 63.0, 26.1, 26.0, 18.5, 18.2, −4.1, −4.3, −5.2, −5.2.

### Step 2: Synthesis of N-((2S,3S,4R,5R)-4-((tert-butyldimethylsilyl)oxy)-5-(((tert-butyldimethylsilyl)oxy)methyl)-3-fluorotetrahydrofuran-2-yl)-N-(phenylsulfonyl) benzenesulfonamide (3-TBS)

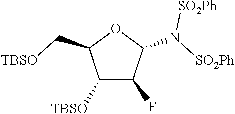

In a 1 L flask was charged the crude 2-TBS (1.0 equiv, 82.95 g, 166 mmol) and CPME (263 mL). Additionally, 2,4,6-trimethylpyridine (4.53 mL, 34.2 mmol), BSTFA (22.02 mL, 83 mmol), and NFSI (68.0 g, 216 mmol) were added to the reaction mixture. The resulting mixture was warmed to 65° C. and stirred for 20 h. After cooling to ambient temperature, heptane (286 mL) was added, and the reaction mixture was stirred for 1.75 h at ambient temperature. The resulting slurry was filtered, and the cake was washed with CPME:heptane (1:1, 286 mL). The filtrate was subsequently concentrated under vacuum. Heptane (286 mL) was added to the concentrated crude material, and the mixture was heated to 70° C. The mixture was filtered while hot into a 1 L flask, and the filtrate was crystallized while being slowly cooled to ambient temperature. The resulting slurry was further cooled to −30 to −35° C. and filtered. After drying under vacuum, the desired DBSI adduct 3-TBS (94.63 g, 138 mmol) was collected.

¹H NMR (500 MHz, CDCl₃) δ 8.04 (dd, J=8.5, 1.1 Hz, 4H), 7.64 (t, J=7.5 Hz, 2H), 7.54 (t, J=7.9 Hz, 4H), 6.00 (dd, J=16.5, 5.9 Hz, 1H), 5.67 (ddd, J=57.2, 6.3, 6.3 Hz, 1H), 4.48 (ddd, J=21.3, 8.9, 6.7 Hz, 1H), 4.39-4.24 (m, 1H), 3.78 (ddd, J=12.0, 1.8, 1.8 Hz, 1H), 3.65 (dd, J=12.0, 3.1 Hz, 1H), 0.92 (s, 9H), 0.88 (s, 9H), 0.10 (s, 3H), 0.08 (s, 3H), 0.05 (s, 3H), 0.05 (s, 3H).

¹³C NMR (126 MHz, CDCl₃) δ 140.6, 134.0, 129.1, 128.4, 99.7 (d, J=188.1 Hz), 92.2 (d, J=36.8 Hz), 83.4 (d, J=9.9 Hz), 73.0 (d, J=20.9 Hz), 61.3, 26.0, 25.7, 18.5, 18.0, −4.5, −5.0, −5.1, −5.3.

¹⁹F NMR (500 MHz, CDCl₃) δ −195.0.

### Step 3: Synthesis of 1-((2R,4S,5R)-4-((tert-butyldimethylsilyl)oxy)-5-(((tert-butyldimethylsilyl) oxy) methyl)tetrahydrofuran-2-yl)-5-methylpyrimidine-2,4(1H,3H)-dione

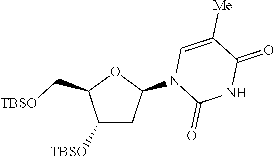 [SMILES: Cc1cn([C@H]2C[C@H](O[Si](C)(C)C(C)(C)C)[C@@H](CO[Si](C)(C)C(C)(C)C)O2)c(=O)[nH]c1=O]

While under a nitrogen atmosphere, thymidine (12.1 g, 50 mmol), imidazole (2.5 equiv, 8.5 g, 125 mmol), tert-butyldimethylsilyl chloride (2.2 equiv, 16.6 g, 110 mmol), DMF (20 mL), and DMAP (0.01 equiv, 0.061 g, 0.5 mmol) were added to a 200 mL round-bottom flask, and the resulting mixture was stirred for 1 h at ambient temperature. The reaction was determined to be complete by HPLC. Subsequent addition of 100 mL water was followed by stirring at ambient temperature for 1 h. Filtration of the slurry was performed, and the cake was washed with 200 mL water. The cake was dissolved in 100 mL MTBE, and the solution was washed with 100 mL water and dried over magnesium sulfate. The filtered MTBE solution was evaporated to approximately 30 mL, diluted with 30 mL hexanes and 80 mL heptane and evaporated to approximately 100 mL. The residue was cooled to 0° C. over 2 h, and crystallization was observed to occur. The slurry was filtered and washed with 30 mL 9:1 hexanes:MTBE and subsequently with 50 mL hexanes. The solid was dried under a nitrogen stream to provide 1-((2R,4S,5R)-4-((tert-butyldimethylsilyl) oxy)-5-(((tert-butyldimethylsilyl)oxy)methyl) tetrahydrofuran-2-yl)-5-methylpyrimidine-2,4(1H,3H)-dione (21 g, 44.6 mmol).

¹H NMR (500 MHz, CDCl₃) δ 8.35 (s, 1H), 7.47 (d, J=1.2 Hz, 1H), 6.33 (dd, J=7.9, 5.8 Hz, 1H), 4.40 (ddd, J=5.6, 2.5, 2.5 Hz, 1H), 3.93 (ddd, J=2.5, 2.5, 2.5 Hz, 1H), 3.87 (dd, J=11.4, 2.6 Hz, 1H), 3.76 (dd, J=11.4, 2.4 Hz, 1H), 2.25 (ddd, J=13.1, 5.8, 2.6 Hz, 1H), 2.00 (ddd, J=13.3, 7.9, 6.1 Hz, 1H), 1.92 (d, J=1.1 Hz, 3H), 0.93 (s, 9H), 0.89 (s, 9H), 0.11 (s, 3H), 0.11 (s, 3H), 0.08 (s, 3H), 0.07 (s, 3H),

¹³C NMR (126 MHz, CDCl₃) δ 164.3, 150.7, 135.8, 111.2, 88.2, 85.2, 72.6, 63.4, 41.8, 26.3, 26.1, 18.8, 18.4, 12.9, −4.3, −4.5, −5.0, −5.1.

### Step 4: Synthesis of Piv-protected (2R,3R,4R,5R)-5-(6-amino-9H-purin-9-yl)-4-fluoro-2-(hydroxymethyl)tetrahydrofuran-3-ol

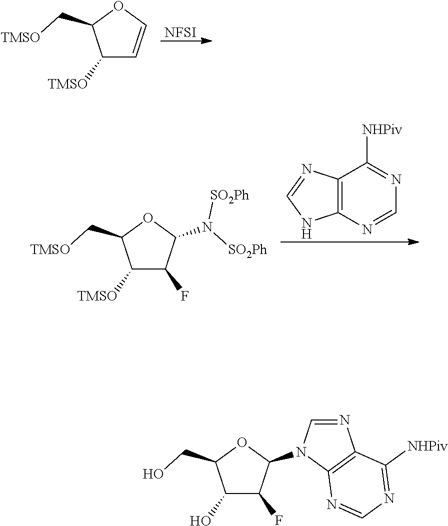

To a 100 L reactor was charged the crude toluene stream for trimethyl(((2R,3S)-3-((trimethylsilyl)oxy)-2,3-dihydrofuran-2-yl)methoxy)silane (10.2 kg, 13.4 mol), which contained 0.36 equivalents lutidine and 2 vol toluene. To this was added toluene (8.75 L), 2,6-lutidine (0.563 L, 4.84 mol), and BSTFA (0.178 L, 0.672 mol), and the resulting mixture was warmed to 65° C. N-fluorobenzenesulfonimide (NFSI) (4.66 kg, 14.77 mol) was added portionwise, then toluene (1.75 L) was used to rinse the sides of the reactor. The reaction mixture was stirred at 65° C. until trimethyl(((2R,3S)-3-((trimethylsilyl)oxy)-2,3-dihydrofuran-2-yl)methoxy)silane was consumed judged by NMR analysis, after which 2,6-lutidine (0.782 L, 6.72 mol), ethyl acetate (50.75 L), and N-(9H-purin-6-yl)pivalamide (2.88 kg, 12.76 mol) were added. An additional 1.75 L of ethyl acetate was used to rinse the sides of the reactor. The resulting mixture was warmed to 75° C. and stirred for overnight. The crude reaction mixture was then concentrated under vacuum to a total volume of 35 L. The resulting slurry was filtered, and the filter cake was washed with ethyl acetate (17.5 L, 5 vol). The filtrate was transferred to a 50 L reactor while continuously evaporating under vacuum to a total volume of 17.5 L. To this, ethanol (5.25 L), 2,6-lutidine (0.313 L, 2.69 mol), and TFA (103 ml, 1.34 mol) were added to start desilylation. Vacuum distillation while feeding 21 L of 3.8:1 v/v EtOAc:EtOH was conducted to aid the desilylation. When the desilylation was achieved with >90% conversion 17.5 L EtOAc was fed into the reactor while distilling away excess EtOH. An additional continuous vacuum distillation was performed with 3.5 L toluene feed while the mixture was concentrated to the total volume of 17.5 L. After the distillation was completed, the reaction mixture was stirred at room temperature overnight to crystallize. The product was collected by filtration rinsing with 21 L of 2:10:88 v/v/v EtOH:tol:EtOAc. Total mass was 3.16 kg.

¹H NMR (500 MHz, DMSO-d6) δ 10.19 (s, 1H), 8.72 (s, 1H), 8.56 (d, J=1.9 Hz, 1H), 7.30-7.10 (toluene, m, 5H), 6.55 (dd, J=13.4, 4.7 Hz, 1H), 5.99 (bs, 1H), 5.29 (ddd, J=52.6, 4.3, 4.3 Hz, 1H), 4.49 (ddd, J=19.1, 4.6, 4.6, 1H), 3.89 (ddd, J=4.8, 4.8, 4.8 Hz, 1H), 3.72 (dd, J=12.1, 3.1 Hz, 1H), 3.66 (dd, J=12.0, 5.1 Hz, 1H), 2.30 (toluene, s, 3H), 1.28 (s, 9H).

¹³C NMR (124 MHz, DMSO-d6) δ 176.3, 151.8, 151.7, 150.4, 142.8 (d, J=3.8 Hz), 137.4 (toluene), 128.9 (toluene), 128.2 (toluene), 125.2 (toluene), 125.1, 95.4 (d, J=192.4 Hz), 83.5 (d, J=5.7 Hz), 81.5 (d, J=17.0 Hz), 72.5 (d, J=23.0 Hz), 60.3, 26.9, 21.0 (toluene).

¹⁹F NMR (470 MHz, DMSO-d6) δ 197.9.

### Step 5: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate from (2R,3R,4R,5R)-5-(6-amino-9H-purin-9-yl)-4-fluoro-2-(hydroxymethyl)tetrahydrofuran-3-ol

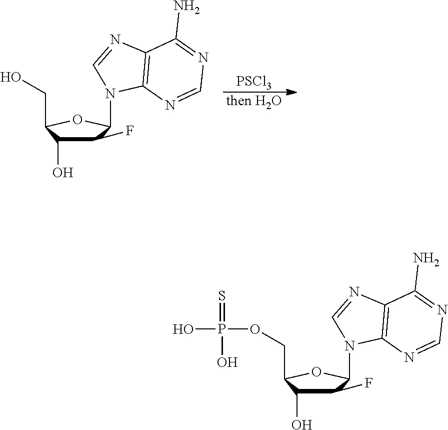

(2R,3R,4R,5R)-5-(6-amino-9H-purin-9-yl)-4-fluoro-2-(hydroxymethyl) tetrahydrofuran-3-ol (50 g, 186 mmol) was azeotroped with 3×100 mL of dry pyridine and was dissolved in 500 mL (10 vol) of dry pyridine (KF=128 ppm). The pyridine solution was cooled to 0° C. for 1 h. Thiophosphoryl chloride (1.04 eq) was added dropwise at 0° C. over 10 min. The reaction was stirred at 0° C. for 80 min, with constant monitoring by UPLC. The reaction was filtered to remove the excess starting material. Water (10 eq) was then added to the filtrate at 0° C. and was slowly warmed to room temperature. The reaction was allowed to stir for an additional 30 min at room temperature. The volatiles were removed in vacuo, and the product was dissolved in 500 mL of water. The solution pH was 4. The solution was filtered, and the filtrate was stirred while 12M HCl was added until the pH of the solution was 0 (about 35 mL). The resulting slurry was allowed to stir at room temperature overnight (˜16 h). Then the slurry was allowed to settle for 1 h. The slurry was then filtered, and the filter cake was washed with 200 mL of water. The washed cake was allowed to dry over a stream of nitrogen overnight (29.9 g).

¹H NMR (500 MHz, DMSO-d₆) δ=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H).

³C NMR (125 MHz, DMSO-d₆): δ=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz).

¹⁹F NMR (470 MHz, DMSO-d₆): δ=−197.80 (ddd, J=52.7, 16.6, 16.6 Hz, 1F).

³¹P NMR (202 MHz, DMSO-d₆): δ=59.49 (dd, J=7.4, 7.4 Hz, 1P).

### Step 5, alternate route: Synthesis of (O-{[(2R,3R,4S,5R)-5-(6-amino-9H-purin-9-yl)-4-fluoro-3-hydroxyoxolan-2-yl]methyl}O,O-dihydrogen phosphorothioate from Piv-protected (2R,3R,4R,5R)-5-(6-amino-9H-purin-9-yl)-4-fluoro-2-(hydroxymethyl)tetrahydrofuran-3-ol

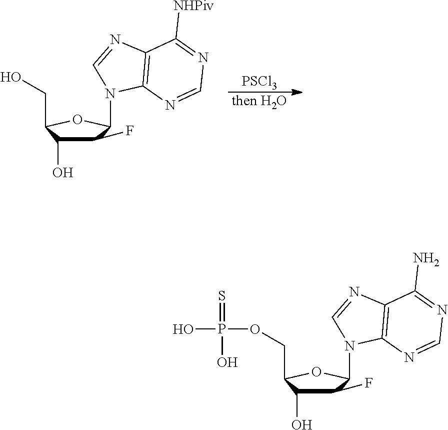

In a 50 L reactor was charged triethylphosphate (4 vol, 8.58 L), Piv-protected (2R,3R,4R,5R)-5-(6-amino-9H-purin-9-yl)-4-fluoro-2-(hydroxymethyl)tetrahydrofuran-3-ol (2.5 kg, 85.75 wt %) followed by the remaining triethylphosphate (1 vol, 2.14 L) washing the sides of the vessel. To this, 2,6-lutidine (3 eq, 1.97 kg) and pyridine (0.3 eq, 144 g) were charged, and the resulting mixture was cooled to −20° C. Then thiophosphoryl chloride (1.835 kg, 1.75 eq.) was added slowly over 1 h. The reaction mixture was aged at −20° C. for overnight, after which additional thiophosphoryl chloride (32 mL, 0.05 eq.) was added. Water (8 eq, 0.87 L) was added dropwise over 1 h to quench the reaction. Additional water (32 eq, 3.5 L) was added dropwise over 1 h, then the resulting mixture was warmed to 50° C. and aged at that temperature for 3 h. After pivaloyl group was removed judged by HPLC analysis, the mixture was cooled to 30° C., and added water (9 vol, 19.3 L) to crystallize the product while cooling to 0° C. slowly. The product was collected by filtration rinsing with water (12.5 L) then dried under vacuum with nitrogen sweep. The resulting product (1.971 kg, 88.65 wt %) was then collected and stored under ambient temperature.

¹H NMR (500 MHz, DMSO-d₆) δ=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H).

¹³C NMR (125 MHz, DMSO-d₆): δ=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz).

¹⁹F NMR (470 MHz, DMSO-d₆): δ=−197.80 (ddd, J=52.7, 16.6, 16.6 Hz, 1F).

³¹P NMR (202 MHz, DMSO-d₆): δ=59.49 (dd, J=7.4, 7.4 Hz, 1P).

### Example 4: Synthesis of 2′F-ThioATP from (O-{[(2R,3R,4S,5R)-5-(6-amino-9H-purin-9-yl)-4-fluoro-3-hydroxyoxolan-2-yl]methyl}O,O-dihydrogen phosphorothioate

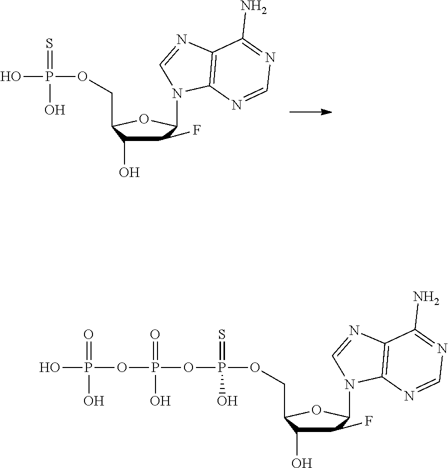

To a 1 L reactor was charged 2′F-thio-AMP (23.3 g, 86 wt %), 2′F-thio-ATP disodium salt, tetrahydrate (1 g, 85 wt %) and 0.9M aq. solution of acetyl phosphate, disodium (3.6 eq, 219 mL). The reaction solution was added 1M aq solution of MgCl₂·(H₂O)₆ solution (0.125 eq, 6.9 mL), and the pH of the reaction mixture was adjusted to 6.5 with addition of NaOH. The reaction volume was diluted to 500 mL with water. An adenylate kinase enzyme that has the amino acid sequence that is SEQ ID NO: 27 (100 mg) and a kinase enzyme that has the amino acid sequence that is SEQ ID NO: 22 (200 mg) were charged to the reaction vessel, and the reaction mixture was stirred at 500 rpm at ambient temperature. After 6 h, the reaction was quenched with 37% aq. solution of HCl (40 mL) to bring the pH to 2. The resulting slurry was filtered, and the filtrate was transferred into 3 L vessel with an overhead stirrer rate of 270 rpm. The filtered solution was charged sodium chloride (2.0 eq, 6.41 g). EtOH (505 mL) was charged to the reaction mixture, and 2′F-ThioATP, disodium salt, tetrahydrate was added as seeds. Once seed bed is formed, the crystal slurry was stirred overnight at 270 rpm. After overnight aging, the slurry was charged another portion of EtOH (130 mL) over 2 h via addition funnel. The reaction vessel was cooled to 4° C. Another portion of EtOH (500 mL) was charged over 4 h via addition funnel to reach EtOH/water ratio of approximately 2:1. The slurry was filtered, and the wet cake was washed with cold 2:1 EtOH/water solution (4×20 mL), cold EtOH (3×20 mL). The resulting wet cake was dried under vacuum with positive nitrogen pressure overnight to yield 2′F-ThioATP (31.3 g).

¹H NMR (500 MHz, Deuterium Oxide) δ 8.67 (d, J=1.8 Hz, 1H), 8.49 (s, 1H), 6.60 (dd, J=11.7, 4.8 Hz, 1H), 5.40 (dt, J=51.7, 4.6 Hz, 1H), 4.81 (m, 1H), 4.45-4.38 (m, 2H), 4.35 (m, 1H).

¹³C NMR (126 MHz, Deuterium Oxide) δ 151.0 147.9, 146.8, 142.7 (d, J=3.7 Hz), 117.9, 94.3 (d, J=193.8 Hz), 82.5 (d, J=17.1 Hz), 81.7 (dd, J=9.4, 5.8 Hz), 72.2 (d, J=24.6 Hz), 64.3 (d, J=6.3 Hz).

³¹P NMR (203 MHz, Deuterium Oxide) δ 43.92 (d, J=27.0 Hz), −10.88 (d, J=19.4 Hz), −23.94.

### Example 5: Preparation of Cobalt-Treated cGAS

500 mL of cGAS whole cell lysate was spun at 5000 G-force at 4° C. for 20 min. The supernatant was discarded, and the insoluble fraction was suspended with 500 mL (1 vol) of ultrapure, deionized, biology-grade water. The resulting mixture was spun at 5000 G-force at 4° C. for 20 min. The resulting supernatant was discarded, and the insoluble fraction was suspended with 500 mL of 0.1M CoSO₄ (1 vol, pH 4-8). The mixture was incubated for 1 h at RT. The resulting mixture was spun at 5000 G-force at 4° C. for 20 min. The resulting supernatant was discarded, and the insoluble fraction was suspended with 500 mL of ultrapure, deionized, biology-grade water (1 vol). The resulting mixture was spun at 5000 G-force at 4° C. for 20 min.

The resulting supernatant was discarded, and the insoluble fraction is Co-treated cGAS, which was stored at 4° C. and used directly for the cGAS reaction.

### Example 6: Tandem Synthesis of 2′F-Thio-ATP and 3′F-Thio-GTP from Monophosphate Precursors Using Co-Immobilized Enzymes

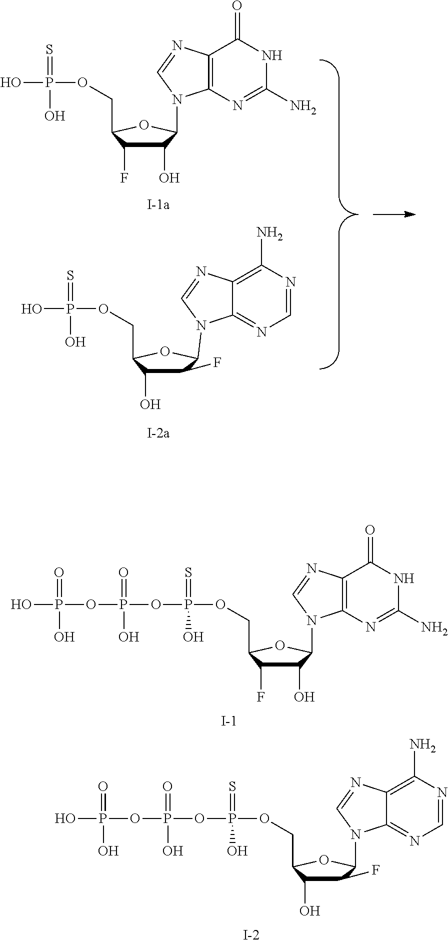

### Step 1: Immobilization

Ni-functionalized chelating resin suspension (commercially available as Bio-rad Nuvia IMAC Ni, 1.8 L, 53 vol % resin solids in 20%/80% EtOH/water) was added to a filter and washed (10 L) with binding buffer (50 mM sodium phosphate buffer; 500 mM NaCl, pH 8) to remove the resin storage solution. The resin was isolated as a cake by filtration, and then re-suspended in the binding buffer (0.75 L) and transferred by funnel into a first reactor (10 L). An addition 0.25 L of binding buffer was used to rinse the transfer vessel, and this liquid was also transferred into the first reactor.

In a second vessel, lyophilized crude cell-free extracts were charged at a pre-determined ratio: a kinase enzyme that has the amino acid sequence that is SEQ ID NO: 19 (21.20 g), a kinase enzyme that has the amino acid sequence that is SEQ ID NO: 27 (16.90 g), and a kinase enzyme that has the amino acid sequence that is SEQ ID NO: 22 (12.70 g), and the extracts were dissolved in binding buffer (1.0 L). Following complete dissolution, the contents of the second vessel were charged into the first reactor and aged overnight at 4° C. with overhead agitation. The resulting mixture was filtered over vacuum yielding a wet cake of immobilized-enzyme on resin. The resulting cake was subsequently washed with 10 L of a modified binding buffer containing imidazole (50 mM sodium phosphate buffer; 500 mM NaCl; 15 mM imidazole, pH 8) and then washed with water (10 L). The washed resin was isolated as a wet cake by filtration, re-suspended in water (1.0 L), and stored at 4° C. prior to use.

### Step 2: Reaction

In a third reactor (100 L), the following material was charged and held at 25° C.: 25 L water, followed by 3′F-thio-GMP (600 g, 1.0 eq), followed by 1.0 L water to rinse the vessel walls. The mixture was dissolved with overhead agitation and subsequently cooled to 10° C. To the third reactor, 2′F-thio-AMP (283 g, 0.87 eq), 2′F-thio-ATP (5.29 g, 0.01 eq), dilithium acetylphosphate (609 g, 88 wt % purity, 4.5 eq), MgCl₂ 5H₂O (374 g, 2.0 eq), and KCl (68.6 g, 1.0 eq) were charged, followed by water (1.0 L) to rinse the walls of the vessel. The resulting mixture was held at 10° C. and briefly agitated. The pH of the solution was then adjusted to approximately 7.3-7.4 using conc. KOH and HCl (5.0 N). Water was added to adjust the final fill volume to 28.15 L.

While continuing to agitate the third reactor, 15% of the immobilized enzyme prepared in Step 1 was aliquoted into a bottle and stored at 4° C., while the remaining 85% of the immobilized enzyme was added to the 50 L reactor, including 500 mL water used to rinse the vessel in which the immobilized enzyme was stored. An additional 500 mL water was added to the reactor to rinse the vessel walls. The mixture was aged for 22 h at 10° C. After the reaction was judged complete by HPLC analysis, the vessel contents were emptied into a filter, and the reaction filtrate was isolated under gentle vacuum and stored at 4° C. or −20° C. for subsequent use.

### Example 7: Tandem Synthesis of 2′F-Thio-ATP and 3′F-Thio-GTP from Monophosphate Precursors Using Independently Immobilized Enzymes

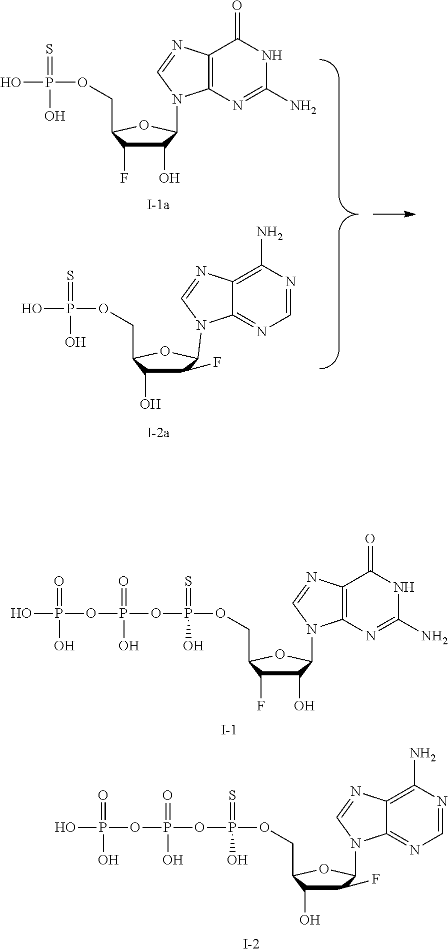

### Step 1: Immobilization

Ni-NTA resin (commercially available as Bio-rad Nuvia IMAC Ni, 2.14 mL of 70 vol % resin slurry) was transferred to a filter, and the storage solution removed by vacuum filtration. Subsequently, the resin was displacement washed with a total of 15 mL binding buffer (50 mM sodium phosphate buffer; 500 mM NaCl, pH 8), resuspended in 3.0 mL binding buffer and transferred to a centrifuge tube, yielding a 50 vol % suspension of resin in binding buffer.

Lyophilized CFE powders of a kinase enzyme that has the amino acid sequence that is SEQ ID NO: 19, a kinase enzyme that has the amino acid sequence that is SEQ ID NO: 27, and a kinase enzyme that has the amino acid sequence that is SEQ ID NO: 22 were separately immobilized as follows: 25 mg of the respective lyophilized CFE was weighed into a vial and resuspended in 0.5 mL binding buffer. To each 1.0 mL of the 50 v % suspension of Ni-NTA resin prepared above was added, followed by an additional 1.0 mL binding buffer. Each vial was closed and mixed at RT for 1 h to complete the immobilization.

Subsequently, the immobilized enzyme-resin from each vial was isolated as follows: the supernatant was decanted, and the resin was washed with a total of 5.0 mL of a modified binding buffer (50 mM sodium phosphate buffer; 500 mM NaCl, 15 mM imidazole, pH 8) followed by 5.0 mL of 1×PBS, the supernatant was decanted, and the resin was resuspended in 1.5 mL water to obtain a 33 vol % slurry of immobilized enzyme resin in water.

### Step 2: Reaction

A reaction master mix was created by charging the following to a vessel: 2′F-Thio-ATP (9.45 mg, 0.05 eq), 2′F-Thio-AMP (111 mg, 0.87 eq), 3′F-Thio-GMP (200 mg, 1.0 eq), dilithium acetyl phosphate (207 mg, 4.25 eq), water (8.0 mL), 1M MgCl 0.6H₂O (604 μL, 2 eq). The pH was adjusted to 7.47 by addition of 2N KOH (145 μL, 0.98 eq) and brought up to 10.0 mL with water. The stock solution was stored at 4° C. until ready for use.

Reactions were performed in a 96-well deep well microplate. To each well, 500 μL of the reaction master mix was added. The reaction stoichiometry for each experiment was varied by changing the volume of each immobilized enzyme resin charged into the wells, between 0.1 μL and 5.0 μL of each resin.

The plate was sealed and mixed on a thermomixer at 10° C. The reaction progress was assessed at both 16 h and 24 h time points. For each, the reaction mixture was sampled, diluted volumetrically 20× with an aqueous solution containing 25% acetonitrile, and the conversion was analyzed by UPLC.

### Example 8: Synthesis of [P(R)]-2′-deoxy-2′-fluoro-5′—O—[(R)-hydroxymercaptophosphinyl]-P-thio-p-D-arabino-adenylyl-(3′→5′)-3′-deoxy-3′-fluoroguanosine cyclic nucleotide using isolated 2′F-thio-ATP and 3′F-thio-GTP

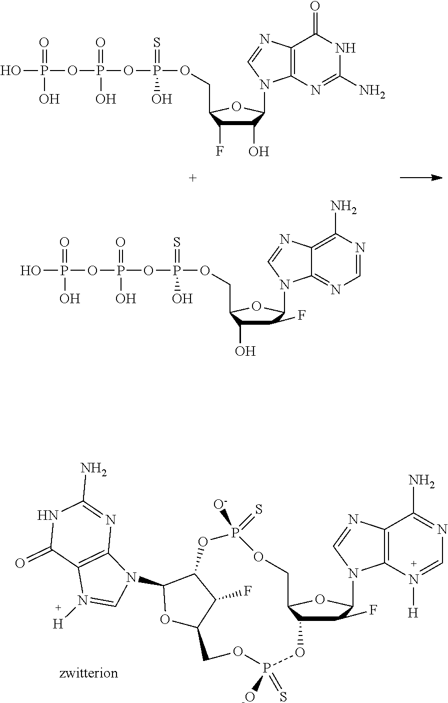

To a flask at RT under N₂ were charged 10 mL of TES (1M, 5 mmol, pH=7.5), 6 mL of 3′F-thio-GTP (0.1M, 0.3 mmol, pH=7), 1.82 mL of 2′F-thio-ATP (0.33M, 0.3 mmol, pH=7), 3 mL of CoSO₄ (0.5M, 0.75 mmol), 3 mL of ZnSO₄ (0.5M, 0.75 mmol), 10 mL of TGDE. This solution was warmed up to 35° C., and the pH was adjusted to 7.4 via 0.1N KOH solution. A wet cGAS pellet (872 mg, 15 wt % cGAS) in 8 mL of DI water was charged, and reaction mixture was aged at 35° C. for 24 h. The reaction was then quenched with NaH₂PO₄ and cooled down to RT.

### Example 9: Synthesis of [P(R)]-2′-deoxy-2′-fluoro-5′—O—[(R)-hydroxymercaptophosphinyl]-P-thio-p-D-arabino-adenylyl-(3′→5′)-3′-deoxy-3′-fluoroguanosine cyclic nucleotide from 2′F-thio-AMP and 3′F-thio-GMP prepared using immobilized kinases

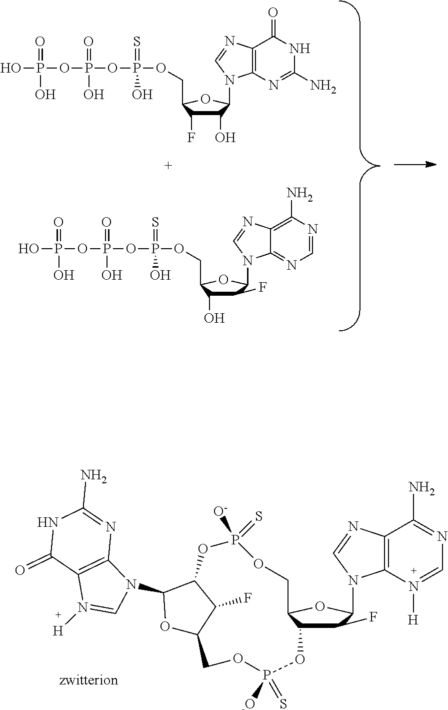

To a 100 L vessel was charged 31 L of a solution containing 2′F-thioATP (0.58 mol) and 3′F-thioGTP (0.64 mol), followed by water used to rinse the container that was used to store the solution (6 L). The jacket temperature of the vessel was set to 45° C., and the agitation set to 80 RPM. N-[tris(hydroxymethyl)methyl]-2-aminoethanesulfonic acid (TES, 2.148 kg, 9.37 mol) and water used to rinse the TES container (4 L) were added, giving a pH of 6.1. The pH was then adjusted to 8.0 via addition of potassium hydroxide (0.5 L, 45 wt %). TGDE (16 L) was then added, followed by cobalt sulfate solution (1.5M, 1.1 L) and zinc sulfate solution (1.1M, 2 L), along with water used to rinse both containers (2 L). Addition of metal solutions reduced the pH to 7.4. At this time, the jacket temperature was reduced to 42° C.; the reaction temperature was 37° C. Next, cGAS enzyme slurry (8 L) was then added to initiate reaction. The reaction was aged at 35° C. for an additional 13 h until the reaction was judged to have completed (<2% 2′F-thioATP remaining).

### Example 10: Synthesis of [P(R)]-2′-deoxy-2′-fluoro-5′—O—[(R)-hydroxymercaptophosphinyl]-P-thio-P-D-arabino-adenylyl-(3′→5′)-3′-deoxy-3′-fluoroguanosine cyclic nucleotide from 2′F-thio-AMP and 3′F-thio-GMP

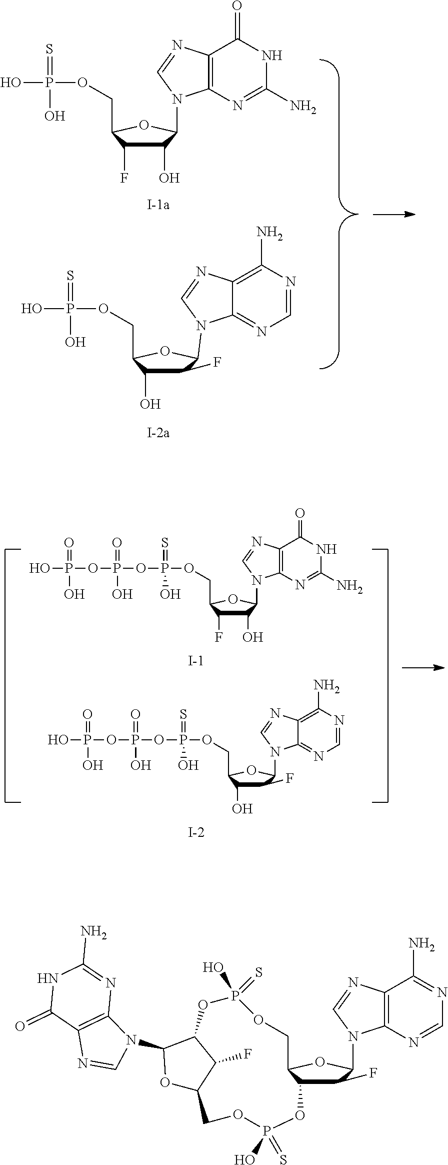

### Step 1

To a 100 L reactor was charged 2′F-thio-AMP (382.2 g, 1.0 eq) and 3′F-thio-GMP (564.7 g, 0.97 eq). The resulting mixture was then cooled down to 10° C.-15° C. followed by addition of water (33.3 L). ATP (57 mg, 0.0001 eq) was dissolved in water (60 mL) and charged to the reactor. To this, MgCl₂·6H₂O (369.2 g, 2.0 eq) was added at 10° C.-15° C., followed by addition of TES (1.041 kg, 5.0 eq). To adjust the pH of the reaction mixture from 5.20 to 5.98 (10° C.-15° C.), around 70.0 mL of KOH (45 wt %) was utilized. Next, AcP—Li/Li (752.4 g, 5.2 eq) was charged at 10° C.-12° C. Once AcP—Li/Li was fully dissolved, around 150 mL-160 mL of KOH (45 wt %) was added to adjust the pH to 7.42 at 9.5° C.-10.5° C. To this clear solution, a solution of a kinase enzyme that can be represented by SEQ ID NO: 22 (2.10 g dissolved in 0.20 L water) was charged, followed by a solution of a kinase enzyme that can be represented by SEQ ID NO: 27 (2.87 g dissolved in 0.25 L water) and a solution of a kinase enzyme that can be represented by SEQ ID NO: 19 (3.44 g dissolved in 0.35 L water) at 9.0° C.-11° C., respectively. The reaction mixture was aged at 10° C. under nitrogen for 17 h-24 h until completion (1-3% 2′F-thio-AMP and 3′F-thio-GMP leftover).

### Step 2

Next, Na₃VO₄ (50.1 g, 0.3 eq) was charged to the reactor, followed by slow addition of a pre-cooled mixture of TGDE (15.3 L) and water (11.0 L), while maintaining the temperature below 15° C. To this, ZnSO₄·7H₂O (784.0 g, 3.0 eq) was added in one portion. Around 270 ml −285 mL of KOH (45 wt %) was charged to adjust the pH from 6.98 to 7.8 at 10° C. Then, cobalt-treated cGAS enzyme slurry that can be represented by SEQ ID NO: 12 in water (22.1 kg) was charged at 10° C. Temperature was increased to 35° C., and reaction was aged at 35° C. for 15 h-24 h, until completion (<2% 3′F-thio-ATP remaining).

### Example 11: Extraction Isolation of [P(R)]-2′-deoxy-2′-fluoro-5′—O—[(R)-hydroxymercaptophosphinyl]-P-thio-p-D-arabino-adenylyl-(3′→5′)-3′-deoxy-3′-fluoroguanosine cyclic nucleotide

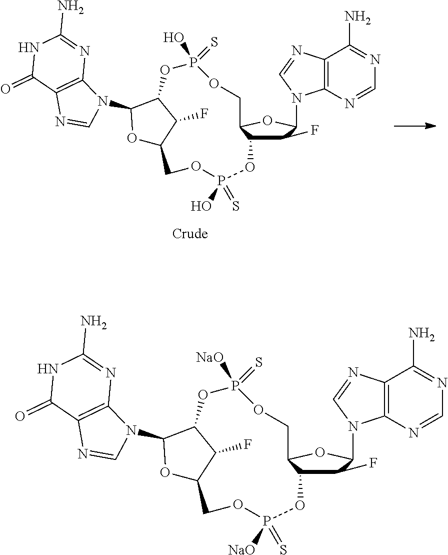

To a 100 L reactor containing 89.00 kg of the cGAS reaction mixture of Example 10 at 49.0° C. was added Na₂SO₄ (1.628 kg, 20 eq). After 2.5 h stirring at 49° C., the reactor was cooled to 10° C., and 43.88 kg of the reaction mixture was removed and stored at 0° C. To the remaining 45.12 kg of reaction mixture was added 65.4 mM [HN(n-oct)_{3]2}[SO₄] in 2-MeTHF (21.9 L, 108 vol., 5.0 eq), and the reaction mixture was stirred for 25 min. The reaction mixture was cooled to −20° C., and 1-propanol (19 L, 93.3 vol.) was added. The reaction mixture was then stirred for 17 h. The reaction mixture was warmed to 50° C. and stirred for 2 h. The cell debris-rich aqueous phase was removed, and the organic phase was filtered to remove residual cell debris. The filtered organic extracts were charged into a 100 L reaction, and 0.25 wt % Na₂SO₄ in water (40 L, 196 vol.) was added. The reaction mixture was stirred for 2 h at RT. The aqueous phase was removed, and the organic phase was stored at 0° C. This step was repeated to recover additional crude product.

The organic extracts were combined in a 100 L reactor at 23° C., and water (6.6 L, 16.2 vol) was added. The mixture was stirred for 25 min. After 25 min, the aqueous phase was removed, water (6.6 L, 16.2 vol) was added, and the mixture was stirred for 25 min. After 25 min, the aqueous phase was removed, and 10% NaCl in water (4 L, 9.8 vol.) was added. The reaction mixture was stirred for 5 min, and the aqueous phase was removed.

The organic extracts were combined in a 30 L reactor at 23° C., and water (500 mL, 1.23 vol) and ION NaOH (585 mL, 1.43 vol., 10.2 eq) were added, until the mixture reached pH 13.15, over 20 min while stirring. The aqueous phase was removed, and 1N NaOH (400 mL, 0.98 vol., 0.70 eq) was added. The reaction mixture was stirred for 10 min, and the aqueous extracts were removed and combined.

The aqueous extracts were filtered through a 1p m filter and added to a 10 L reactor. The aqueous extracts were heated to 55° C. 2N HCl (400 mL, 0.98 vol., 1.40 eq) was added dropwise over 2 h to pH 7.30. The resulting slurry was cooled to 25° C. and stirred for 12 h. The product was collected by filtration and washed once with 93% EtOH:7% water (4 L, 9.82 vol.), and again with 93% EtOH:7% water (1.5 L, 3.68 vol.). The product was dried under air flow for 90 min then under vacuum, at a relative humidity of 32.9% to 45.0%, over 41 h.

¹H NMR (600 MHz, Deuterium Oxide) δ 8.41 (s, 1H), 8.37 (s, 1H), 8.11 (s, 1H), 6.68 (dd, J=15.0, 4.1 Hz, 1H), 6.18 (d, J=8.6 Hz, 1H), 5.90-5.66 (m, 2H), 5.58 (dd, J=53.3, 3.4 Hz, 1H), 5.41 (ddt, J=13.6, 8.3, 3.9 Hz, 1H), 4.86 (d, J=26.0 Hz, 1H), 4.61 (d, J=4.9 Hz, 1H), 4.58 (d, J=8.5 Hz, 1H), 4.31 (t, J=5.8 Hz, 2H), 4.27 (d, J=11.9 Hz, 1H).

It will be appreciated that various of the above-discussed and other features and functions, or alternatives thereof, may be desirably combined into many other different systems or applications. It will also be appreciated that various presently unforeseen or unanticipated alternatives, modifications, variations or improvements therein may be subsequently made by those skilled in the art, and these are also intended to be encompassed by the following claims.

What is claimed is:

1. A process for preparing a compound of Formula (I), or a hydrate, or solvate thereof: 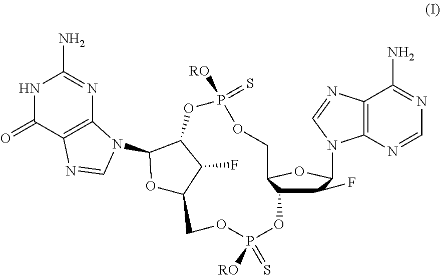
wherein each R is a cation independently selected from the group consisting of H⁺, Na⁺, K⁺, Mg⁺⁺, Co⁺⁺, Zn⁺⁺, and NH₄⁺, said process comprising
reacting a compound of Formula (I-1) with a compound of Formula (I-2), in the presence of at least one cGAS type enzyme:

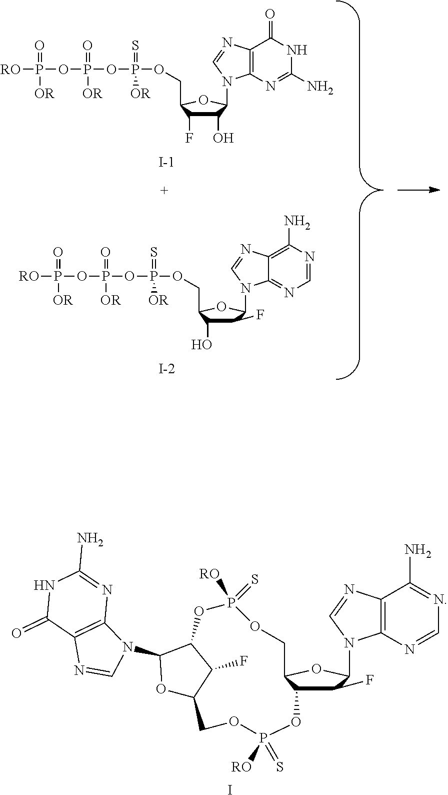

2. The process according to claim 1, wherein the compound of Formula (I) is a compound of Formula (Ia), or a hydrate, or solvate thereof:

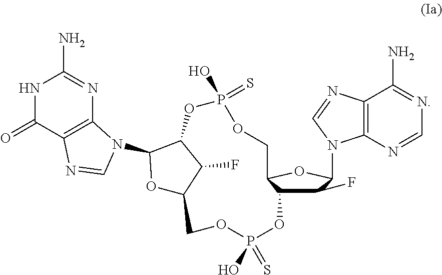 [SMILES: Nc1nc2c(ncn2[C@@H]2O[C@@H]3CO[P@@](O)(=S)O[C@H]4[C@H](F)[C@H](n5cnc6c(N)ncnc65)O[C@@H]4CO[P@@](O)(=S)O[C@@H]2[C@@H]3F)c(=O)[nH]1]

3. The process according to claim 1, wherein the at least one cGAS type enzyme is selected from cGAS type enzymes having an amino acid sequence that is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

4. The process according to claim 1, wherein the at least one cGAS type enzyme is incubated in at least one Chaotropic Agent selected from the group consisting of sodium dodecyl sulfate, thiourea, guanidine HCl, phenol, phenyl acetyl sulfide, urea, KCl, MgCl₂, LiOAc, NaCl, and mixtures thereof.

5. The process according to claim 1, wherein the reacting further comprises reacting in the presence of at least one Metal Co-Factor A.

6. The process according to claim 5, wherein the at least one Metal Co-Factor A is selected from the group consisting of KCl, MgCl₂, ZnSO₄, CoSO₄, CoF₂, Co(SCN)₂, CoBr₂, Co(NO₃)₂, CoCl₂, CoCO₃, Co(C₂O₄)₂, and Co(OH)₂, and mixtures thereof.

7. The process according to claim 1, wherein the reacting is conducted in the presence of at least one Solvent A, where the at least one Solvent A is selected from the group consisting of organic solvents, organic solvents in combination with water, and mixtures thereof.

8. The process according to claim 7, wherein the at least one Solvent A is selected from the group consisting of tetraglyme dimethyl ether, MeCN, MeOH, EtOH, DMSO, propyl nitrile, sulfolane, pyrrolidone, 2-ethoxyl acetate, cyclohexanol, methyl pentyl ketone, cyclohexanone, 1,2,3,4-tetrahydronaphthalene, pivolate methyl ester, 2-methyl-3-butene-2-ol, tert-butanol, DMF, tetra-methyl urea, tetramethylene sulfone, N,N-diethyl acetamide, ethylene glycol, NMP, isopropyl alcohol, 1-methoxy-2-propyl acetate (MPA), and mixtures thereof.

9. The process according to claim 8, wherein the at least one Solvent A is TGDE.

10. The process according to claim 1, further comprising preparing the compound of Formula (I-1) by reacting a compound of Formula (I-1a) with at least one guanylate kinase type enzyme and at least one acetate kinase enzyme:

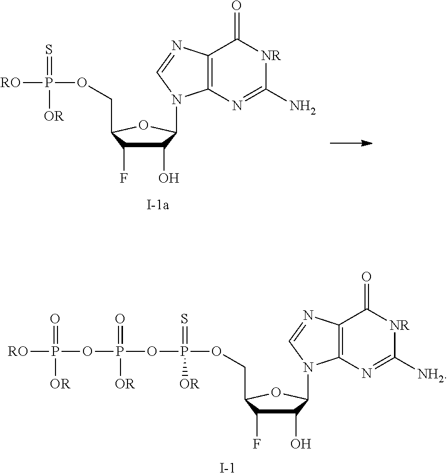

11. The process according to claim 10, wherein the at least one guanylate kinase type enzyme is selected from guanylate kinase type enzymes having an amino acid sequence that is SEQ ID NO: 14, SEQ ID NO: 15, SEQ ID NO: 16, SEQ ID NO: 17, SEQ ID NO: 18, or SEQ ID NO: 19.

12. The process according to claim 10, wherein the at least one acetate kinase type enzyme is selected from acetate kinase type enzymes having an amino acid sequence that is SEQ ID NO: 20, SEQ ID NO: 21, or SEQ ID NO: 22.

13. The process according to claim 10, wherein said reacting is conducted in the presence of at least one Co-Factor A selected from the group consisting of 2′F-thio-ATP and natural ATP.

14. The process according to claim 10, wherein said reacting is conducted in the presence of at least one Metal Co-Factor B selected from the group consisting of MgCl₂, MnCl₂, and Mg(OH)₂, hydrates thereof, and mixtures thereof.

15. The process according to claim 1, further comprising preparing the compound of Formula (I-2) by reacting a compound of Formula (I-2a) with at least one acetate kinase type enzyme and at least one adenylate kinase enzyme:

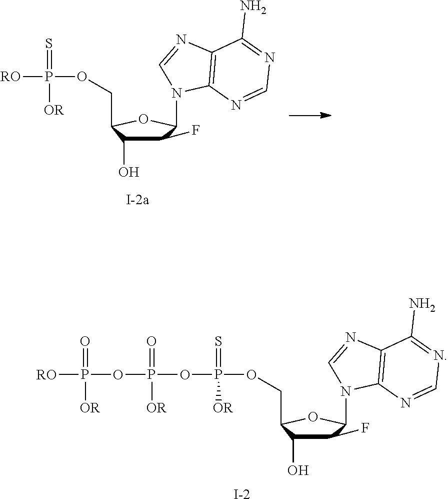

16. The process according to claim 15, wherein the at least one acetate kinase type enzyme is selected from acetate kinase type enzymes having an amino acid sequence that is SEQ ID NO: 20, SEQ ID NO: 21, or SEQ ID NO: 22.

17. The process according to claim 15, wherein the at least one adenylate kinase type enzyme is selected from adenylate kinase type enzymes having an amino acid sequence that is SEQ ID NO: 23, SEQ ID NO: 24, SEQ ID NO: 25, SEQ ID NO: 26, or SEQ ID NO: 27.

18. The process according to claim 15, wherein said reacting is conducted in the presence of at least one Co-Factor A selected from the group consisting of 2′F-thio-ATP and natural ATP.

19. The process according to claim 15, wherein said reacting is conducted in the presence of at least one Metal Co-Factor B selected from the group consisting of MgCl₂, MnCl₂, and Mg(OH)₂, hydrates thereof, and mixtures thereof.

20. The process according to claim 1, further comprising simultaneously preparing the compound of Formula (I-1) by (i) reacting a compound of Formula (I-1a) with at least one guanylate kinase type enzyme and at least one acetate kinase enzyme, and preparing the compound of Formula (I-2) by (ii) reacting a compound of Formula (I-2a) with at least one acetate kinase type enzyme and at least one adenylate kinase enzyme:

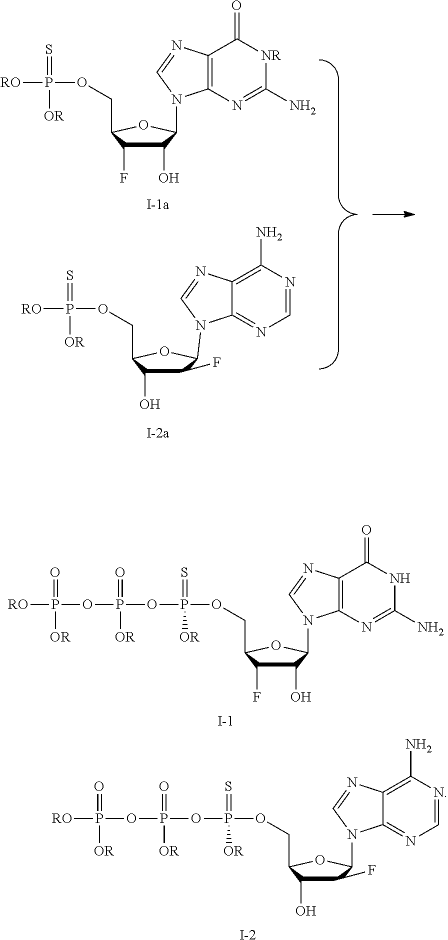

21. The process according to claim 20, wherein the at least one guanylate kinase type enzyme is selected from guanylate kinase type enzymes having an amino acid sequence that is SEQ ID NO: 14, SEQ ID NO: 15, SEQ ID NO: 16, SEQ ID NO: 17, SEQ ID NO: 18, or SEQ ID NO: 19.

22. The process according to claim 20, wherein the at least one acetate kinase type enzyme is selected from acetate kinase type enzymes having an amino acid sequence that is SEQ ID NO: 20, SEQ ID NO: 21, or SEQ ID NO: 22.

23. The process according to claim 20, wherein the at least one adenylate kinase type enzyme is selected from adenylate kinase type enzymes having an amino acid sequence that is SEQ ID NO: 23, SEQ ID NO: 24, SEQ ID NO: 25, SEQ ID NO: 26, or SEQ ID NO: 27.

24. A process for preparing a compound of Formula (I), which comprises 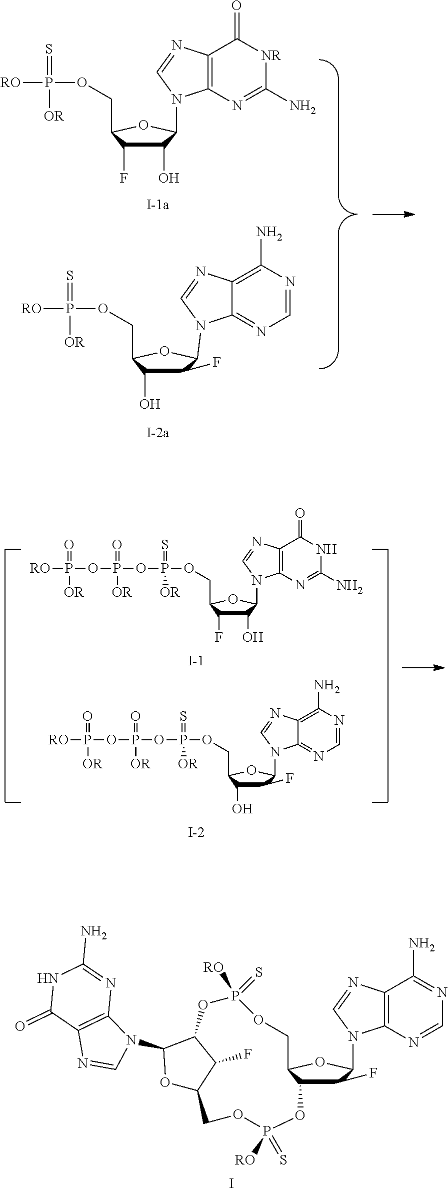
(a) simultaneously preparing the compound of Formula (I-1) by (i) reacting a compound of Formula (I-1a) with at least one guanylate kinase type enzyme and at least one acetate kinase enzyme, and preparing the compound of Formula (I-2) by (ii) reacting a compound of Formula (I-2a) with at least one acetate kinase type enzyme and at least one adenylate kinase enzyme; and
(b) reacting a compound of Formula (I-1) with a compound of Formula (I-2), in the presence of at least one cGAS type enzyme.

25. The process according to claim 24, further comprising
(c) isolating compound I with ammonium salt formation and converting to sodium salt.

26. The process according to claim 24, wherein the at least one guanylate kinase type enzyme is selected from guanylate kinase type enzymes having an amino acid sequence that is SEQ ID NO: 14, SEQ ID NO: 15, SEQ ID NO: 16, SEQ ID NO: 17, SEQ ID NO: 18, or SEQ ID NO: 19.

27. The process according to claim 24, wherein the at least one acetate kinase type enzyme is selected from acetate kinase type enzymes having an amino acid sequence that is SEQ ID NO: 20, SEQ ID NO: 21, or SEQ ID NO: 22.

28. The process according to claim 24, wherein the at least one adenylate kinase type enzyme is selected from adenylate kinase type enzymes having an amino acid sequence that is SEQ ID NO: 23, SEQ ID NO: 24, SEQ ID NO: 25, SEQ ID NO: 26, or SEQ ID NO: 27.

29. The process according to claim 24, wherein the at least one cGAS type enzyme is selected from cGAS type enzymes having the amino acid sequence that is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

30. The process according to claim 25, wherein said isolating comprises extracting, washing, crystallizing, and drying the compound of Formula (I).

31. The process according to claim 30, wherein the process comprises reacting the crude compound of Formula (I) with at least one Salt C selected from the group consisting of Na₂SO₄, NaHSO₄, Na₂CO₃, NaHCO₃, K₂SO₄, KHSO₄, K₂CO₃, KHCO₃, and mixtures thereof.

32. The process according to claim 31, wherein the process further comprises treating the salt mixture with at least one Immiscible Solvent containing at least one Salt D.

33. The process according to claim 32, wherein the at least one Immiscible Solvent is selected from the group consisting of 2-MeTHF, EtOAc, and mixtures thereof.

34. The process according to claim 32, wherein the at least one Salt D is selected from salts having a cation selected from the group consisting of 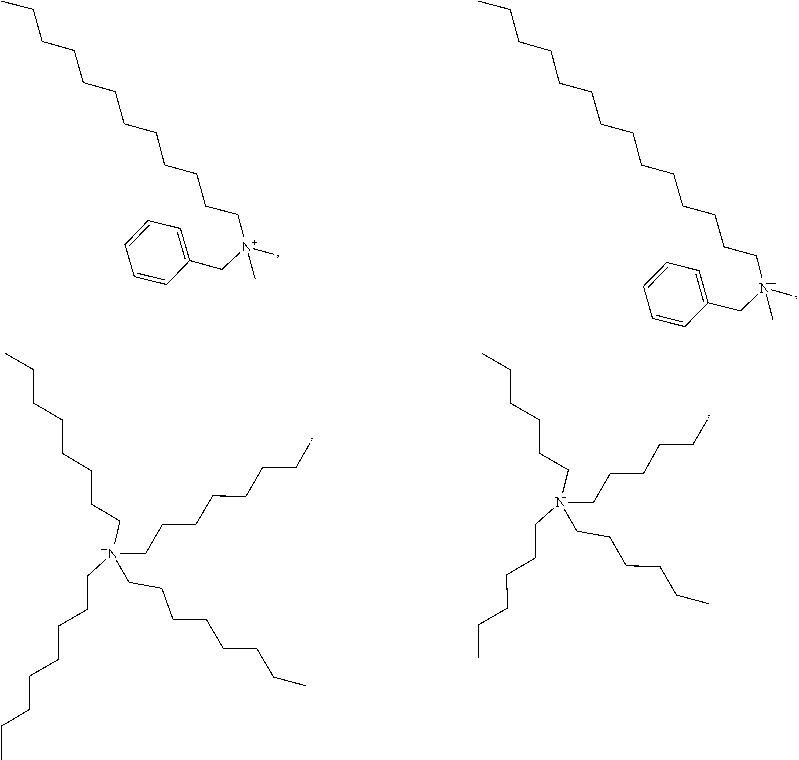 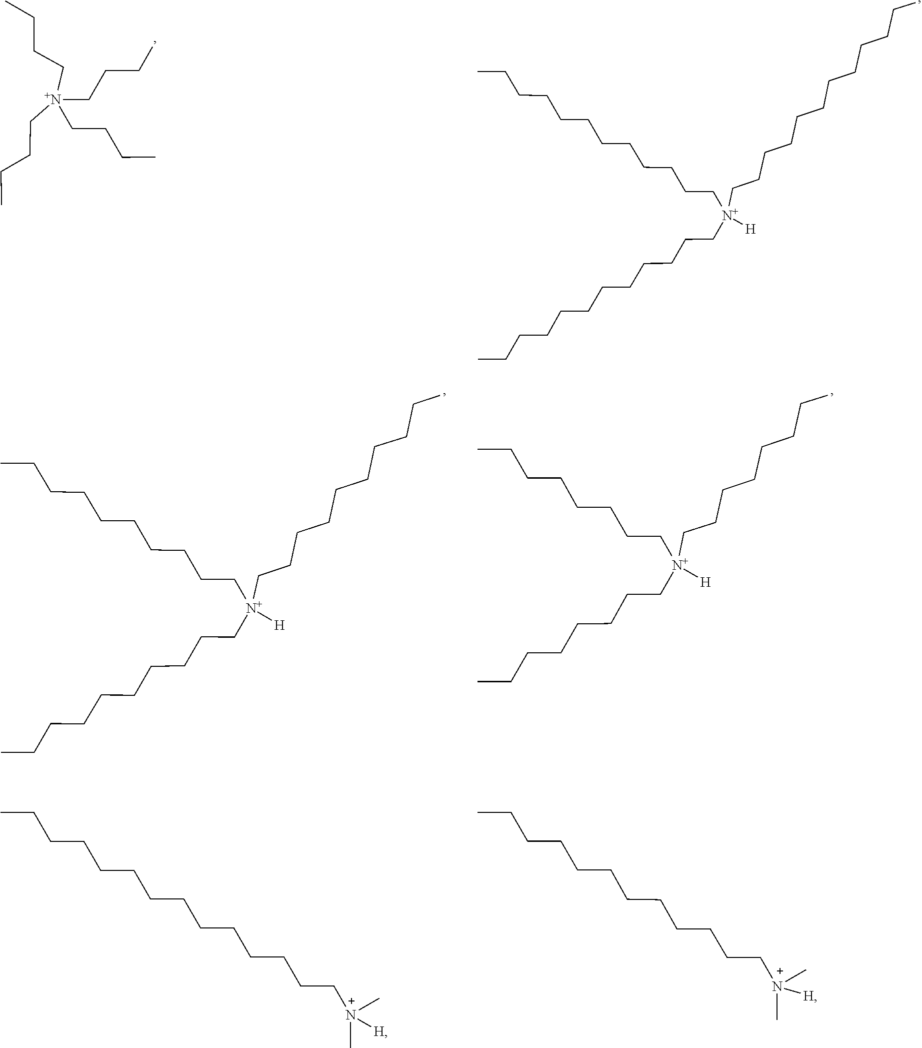 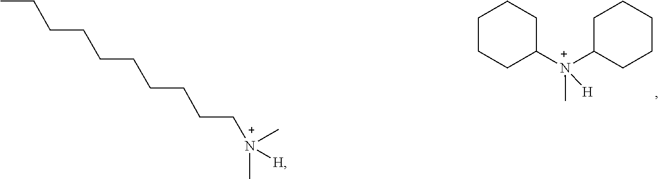
and having an anion selected from Cl⁻, Br⁻, I⁻, HSO₄⁻, SO₄²⁻, H₂PO₄⁻, HPO₄²⁻, PO₄³⁻, and mixtures thereof.

35. A compound selected from the group consisting of 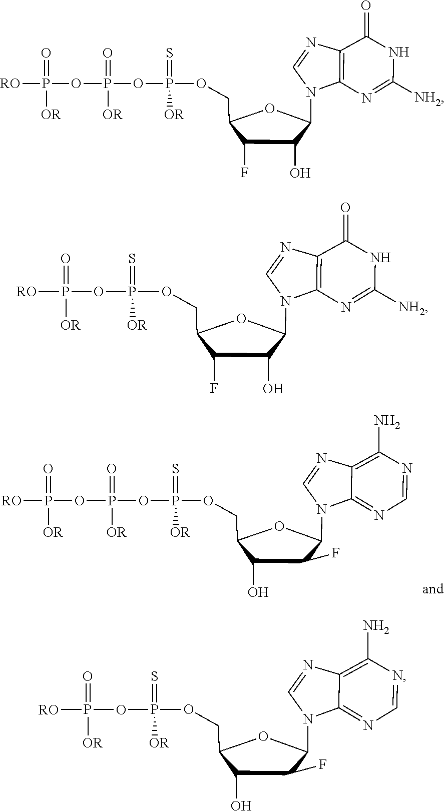
wherein each R is a cation independently selected from the group consisting of H⁺, Na⁺, K⁺, Mg⁺⁺, Co⁺⁺, Zn⁺⁺, and NH₄⁺.


SEQUENCE LISTING (27 sequences)

SEQ ID NO: 1 - protein, 428 aa, Halialeetus leucocephalus
MPSGDWREAVRPRPSSPAVGQEGAGFVSSGEERCVEEGVRPPEAEGGKPRRGSRRPVAAAASSSGGPRLREVLSRLSLGRQDVSEASGLVNQVVSQLIQAIRSQEGSFGSIERLGAGSYYEHVKISEPNEFDIMLVMPVSRLQLDECDDTGAYYYLTFKRNPKEKHLFKFLDEDGKLSAFKMLQALRDIIKREVKNIKNAEVTVKRRKAGSPAITLQIKNPPAEISVDIILTLEVQQNWPPSTQDGLKIEQWLGRKVRGQFRNKSLYLVAKQNKREKVLRGNTWRLSFSHIEKDMLNNHGSSKTCCESDGLKCCRKGCLKLLKYLLEQLKMKYTKQLEKFCSYHVKTAFFHSCVMWPNDTDWHLGDLDYCFQKYLGYFLDCLQKSELPHFFIPQYNLLSLEDKASNDFLSRQINYQLNNRFPIFQERY

SEQ ID NO: 2 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNAGSYYEHVKISEPNEFDIMLVMPVSRLQLDECDDTGAFYYLTFKRNPKEKHLFKFLDEDGKLSAFKMLQALRDIIKREVKNIKNAEVTVKRRKAGSPAITLQIKNPPAEISVDIILTLEVQQNWPPSTQDGLKIEQWLGRKVRGQFRNKSLYLVAKQNKREKVLRGNTWRLSFSHIEKDMLNNHGSSKTCCESDGLKCCRKGCLKLLKYLLEQLKMKYTKQLEKFSSYHVKTAFFHSCVMWPNDTDWHLGDLDYCFQKYLGYFLDCLQKSELPHFFIPQYNLLSLEDKASNDFLSRQINYQLNNRFPIFQERY

SEQ ID NO: 3 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNPKEKHLFKFLDEDGKLSAFKMLQALRDIIKREVKNIKNAEVTVKRKKAGSPAITLQIKNPPAEISVDIILTLEVQQNWPPSTQDGLKIEQWLGRKVRGQFRNKSLYLVAKQNKREKVLRGNTWRISFSHIEKDMLNNHGSSKTCCESDGLKCCRKGCLKLLKYLLEQLKMKYTKQLEKFSSYHVKTAFFHSCVMWPNDTDWHLGDLDYCFQKYLGYFLDCLQKSELPHFFIPQYNLLSLEDKASNDFLSRQINYELNNRFPIFQERY

SEQ ID NO: 4 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNPKEKHLFKFLDEDGKLSAFKMLQALRDIIKREVKNIKNAEVTVKRKKAGSPAITLQIKNPPAEISVDIILTLEVQQSWPPSTQDGLKIEQWLGRKVRGQFRNKSLYLVAKQNKREKVLRGNTWRISFSHIEKDMLNNHGSSKTCCESDGLKCCRKGCLKLLKYLLEQLKMKYTKQLEKFSSYEVKTAFFHSCVMWPNDTDWHLGDLDYCFQKYLGYFLDCLQKSELPHFFIPQYNLLSLEDKASNDFLSRQINYELNNRFPIFQERY

SEQ ID NO: 5 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNPKEKHLFKFLDEDGKLSAFKMLQALRDIIKREVKNIKNAEVTVKRKKAGSPAITLQIKNPPAEISVDIILTLEVQQSWPPSTQDGLKIEQWLGRKVRGQFRNKSLYLVAKQNKREKVLRGNTWRISFSHIEKDMLNNHGSSKTCCESDGLKCCRKGCYKLLKYLLEQLKMKYTKQLEKFSSYEVKTAFFHSCVMWPNDTDWHLGDLDYCFQKYLGYFLDCLQKSELPHFFIPQYNLLSLEDKASNDFLSRQINYELNNRFPIFQERY

SEQ ID NO: 6 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNQKHKHLFKFLDEDGKLSAFKMLQALRDIIKREVKNIKNAEVTVKRKKAGSPAITLQIKNPPAEISVDIILTLEVQQSWPPSTQDGLKIEKWLGRKVRGQFRNKSLYLVAKQNKREKVLRGNTWRISFSHIEKDMLNNHGSSKTCCESDGLKCCRKGCYKLLKYLLEQLKMKYPHQLEKFSSYEVKTAFFHSCVMWPNDSDWHLGDLDYCFQKYLGYFLDCLQKSELPHFFIPQYNLLSLEDKASNDFLSRQINYELNNRFPIFQERY

SEQ ID NO: 7 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEVQQSWPPSTQDGLKIEKWLGRKVRGQFRNKSLYLVAKQNKREKVLRGNTWRISFSHIEKDMLNNHGSSKTCCESDGLKCCRKGCYKLLKYLLERLKMKYPHQLEKRSSYEVKTAFFHSCVMWPNDSDWHLSDLDYCFQKYLGYFLDCLQKSELPHFFIPQYNLLSLEDKASNDFLSRQINYELNNRFPIFQERY

SEQ ID NO: 8 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNSGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEVQQSWPPSTQDGLKIEKWLGRKVRGQFRNKSLYLVAKQNKREKVLRGNTWRISFSHIEKDMLNNHGSSKTCCESDGLKCCRKGCYKLLKYLLERLKMKYPHQLEKRSSYEVKTAFFHSCVMWPNDSDWHLSDLDYCFQKYLGYFLDCLQKSELPHFFIPQYNLLSLEDKASNDFLSRQINYELNNRFPIFQERY

SEQ ID NO: 9 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNSGSYYEHVKISEPNEFDIMLVMPVSRLQLDECDDTGAFYYLTQKRNSKDKHLFKFLDEDGKLSAFKMLQALRDIIKREVKNIKNAEVTVKRKKAGSPAITLQIKNPPAVISVDIILTLEVQQSWPPSTQDGLKIEKWLGRKVRGQFRNKSLYLVAKQNKREKVLRGNTWRISFSHIEKDMLNNHGSSKTCCESDGLKCCRKGCYKLLKYLLERLKMKYPHQLEKRSSYEVKTAFFHSCVMWPNDSDWHLSDLDYCFQKYLGYFLDCLQKSELPHFFIPQYNLLSLEDKASNDFLSRQINYELNNRFPIFQERY

SEQ ID NO: 10 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEPQQSWPPSTQDGLKIEKWLGRKVRGQFRNKSLYLVAKQNKREKVLRGNTWRISFSHIEKDMLNNHGSSKTCCESDGLKCCRKGCYKLLKYLLERLKMKYPHQLEKRSSYEVKTAFFHSCVMWPNDSDWHLSDLDYCFQKYLGYFLDCLQKSELPHFFIPQYNLLSLEDKASNDFLSRQINYELNNRFPIFQERY

SEQ ID NO: 11 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEPQQSWPPSTQDGLKIEKWLGRKVRGQFRNKSLYLVAKQNKREKVLRGNTWRLSFSHIEKDMLNNHGSSKTCCESDGLKCCRKGCYKLLKYLLERLKMKYPHQLEKRSSYEVKTAFFHSCVMWPNDLDWHLSDLDYCFQKYLGYFLDCLQKSELPHFFIPQYNLLSLEDKASNDFLSRQINYELNNRFPIFQERY

SEQ ID NO: 12 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEPQQSWPPSTQDGLKIEKWLGRKVRGQFRNKSLYLVAKQNPREKVLRGNTWRLSFSHIEKDMLNNHGSSKTCCESDGLKCCRKGCYKLLKYLLERLKMKYPHQLEKRSSYEVKTAFFHSCVMWPNDLDWHLSDLDYCFQKYLGYFLDCLQKSELPHFFIPQYNLLSLEDKASNDFLSRQINYELNNRFPIFQERY

SEQ ID NO: 13 - protein, 476 aa, Artificial Sequence
  cGAS
MHHHHHHGSDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGGGPRLREVLSRLSLGRQDVSEASGLVNQVVSQLIQAIRSQEGSFGSIERLNTGSYYEHVKISEPNEFDIMLVMPVSRLQLDECDDTGAFYYLTFKRNSKDKHLFKFLDEDGKLSAFKMLQALRDIIKREVKNIKNAEVTVKRKKAGSPAITLQIKNPPAVISVDIILTLEPQQSWPPSTQDGLKIEKWLGRKVRGQFRNKSLYLVAKQNPREKVLRGNTWRLSFSHIEKDMLNNHGSSKTCCESDGLKCCRKGCYKLLKYLLEQLKMKYPKQLEKRSSYEVKTAFFHSCVMWPNDTDWHLSDLDYCFQKYLGYFLDCLQKSELPHFFIPQYNLLSLEDKASNDFLSRQINYELNNRFPIFQERY

SEQ ID NO: 14 - protein, 476 aa, branchistoma floridae; identical to SEQ ID NO: 13

SEQ ID NO: 15 - protein, 229 aa, Artificial Sequence
  guanylate kinase
MHHHHHHALPRPVVICGPSGSGKSTLYNKLLKEFPGVFQLSVSHTTRQPRPGELNGREYHFINRDQFQENIKQGDFLEWAEFSGNLYGTSKKALEEVQSNNVIPILDIDTQGVRNVKKASLEAVYIFIKPPSIDVLEKRLRSRKTETEEALQKRLSAARNELEYGLKPGNFQHIITNDDLDVAYEKLKGILIKSQMPLAMATGSSSSVVNSFLDKPAASATTVNSSSQD

SEQ ID NO: 16 - protein, 229 aa, Artificial Sequence
  guanylate kinase
MHHHHHHALPTPVVICGPSGSGKTTLYNKLLKEFPGVFQLSVSHTTRQPRPGEENGREFHFINRDQFQENIKQGDFLEWAEFSGNLYGTSKKALEEVQANNVIPILDIDTQGVRNVKKASLEAVYIFIKPPSIDVLEERLRSRKTETEEALQKRLSAARNELEYGLKPGNFQHIITNDDLDVAYEKLKGILIKSQMPLAMATGSSSSVVNSFLDKPAASATTVNSSSQD

SEQ ID NO: 17 - protein, 229 aa, Artificial Sequence
  guanylate kinase
MHHHHHHALPTPVVICGPSGSGKTTLYNKLLKEFPGVFQLSVSHTTRQPRPGEENGREFHFINRDQFQENIKQGDFLEWAEFSGNLYGTSKKALEEVQANNVIPILDIDTQGVRNVKKASLEAVYIFIKPPSIDVLEERLRSRKTETEEALQKRLSAAPNELEYGLKPGNFQHIITNDDLDVAYEKLKGILIKSQMPLAMATGSSSSVVNSFLDKPAASATTVNSSSQD

SEQ ID NO: 18 - protein, 229 aa, Artificial Sequence
  guanylate kinase
MHHHHHHALPTPVVICGPSGSGKTTLYNKLLKEFPGVFQLSVSHTTRQPRPGEENGREFHFINRDQFQENIKQGDFLEWAEHSGNLYGTSKKALEEVQANNVIPILDIDTQGVRTVKKASLEAVYIFIKPPSIDVLEERLRSRGTETEEALQKRLSAAPNELEYGLKPGNFQHIITNDDLDVAYEKLKGILIDSQMPLAGATGSSSSVVNSFLDKPAASATTVNSSSQD

SEQ ID NO: 19 - protein, 229 aa, Artificial Sequence
  guanylate kinase
MHHHHHHALPTPVVICGPSGSGKTTLYNKLLKEFPGVFQLTASHTTRQPRPGEENGREFHFINRDQFQENIKQGDFLEWAEHSGNLYGTSKKALEEVQANNVIPILDIDTQGVRTVKKASLEAVYIFIKPPSIEVLEERLRSRGTETEEALQKRLSAAPNELEYGLKPGNFQHIITNDDLDVAYEKLKGILIDSQMPLAGATGSSSSVVNSFLDKPAASATTVNSSSQD

SEQ ID NO: 20 - protein, 403 aa, Thermotoga maritima
MRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 21 - protein, 412 aa, Artificial Sequence
  acetate kinase
MGSHHHHHGSRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKVAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPTEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 22 - protein, 412 aa, Artificial Sequence
  acetate kinase
MGSHHHHHGSRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKVAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPTEGLVMGTRSGDLDPAIPFFIMEKEGISPQEMYDILNKKSGVYGLSKGFSSDLRDIEEAALKGDEWCKLVLEIYDYRIAKYIGAYAAAMNGVDAIVFTAGVCENSPITREDVCSYLEFLGVKLDKQKNEETIDGKEGIISTPDSRVKVLVVPTNEELMIARDTKEIVEKIGR

SEQ ID NO: 23 - protein, 222 aa, Saccharomyces cerevisiae
MSSSESIRMVLIGPPGAGKGTQAPNLQERFHAAHLATGDMLRSQIAKGTQLGLEAKKIMDQGGLVSDDIMVNMIKDELTNNPACKNGFILDGFPRTIPQAEKLDQMLKEQGTPLEKAIELKVDDELLVARITGRLIHPASGRSYHKIFNPPKEDMKDDVTGEALVQRSDDNADALKKRLAAYHAQTEPIVDFYKKTGIWAGVDASQPPATVWADILNKLGKD

SEQ ID NO: 24 - protein, 228 aa, Artificial Sequence
  adenylate kinase
MHHHHHHSSSESIRMVLIGPPGAGKGTQAPNLQERFHACHLATGDMLRSQIAKGTQLGLEAKKIMDQGGLVSDDIMVNMIKDELTNNPACKNGFILDGFPRTIPQAEKLDQMLKEQGTPLEKAVELKVDDELLVARITGRLIHPASGRSYHKIFNPPKEDMKDDVTGEALVQRSDDNADALKKRLAAYHAQTEPVVDFYKKTGIWAGVDASQPPATVWADILNKLGKD

SEQ ID NO: 25 - protein, 228 aa, Artificial Sequence
  adenylate kinase
MHHHHHHSSSESIRMVLIGPPGAGKGTQAPNLQERFHACHLATGDMLRSQIAKGTQLGLEAKKIMDQGGLVSDDIMVNMIKDELTNNPACKNGFILDGFPRTIPQAEKLDQMLKEQGTPLEKAVELKIDDELLPARITGRLIHPASGRSYHKIFNPPKEDMKDDVTGEALVQRSDDNADALKKRLAAYHKQTEPVVDFYKKTGIWAGVDASQPPATVWADILNKLGKD

SEQ ID NO: 26 - protein, 228 aa, Artificial Sequence
  adenylate kinase
MHHHHHHSSSESIRMVLIGPPGAGKGTQAPNLQERFHACHLGTGDMLRSQIAKGTQLGLEAKKIMDQGGLVSDDIMVNMIKDELTNNPACKNGFILDGFPRTIPQAEKLDQMLKEQGTPLEKAVELKIDDELLPARITGRLIHPASGRSYHKIFNPPKEDMKDDVTGEALVQRSDDNADALKKRLAAYHKQTEPVVDFYKKTGIWAGVDASQPPATVWADILNKLGKD

SEQ ID NO: 27 - protein, 228 aa, Artificial Sequence
  adenylate kinase
MHHHHHHSSSESIRMVLIGPPGAGKGTQSPNLQERFHACHLGTGDMLRSQHAKGTQLGLEAKKIMDQGGLVSDDIMVNMIKDELTNNPACKNGFILDGFPRTIPQAMKLEQMLKEQGTPLEKAVELKIDDELLPARITGRLIHPASGRSYHKIFNPPKEDMKDDVTGEALVQRSDDNADALKKRLAAYHKQTEPVVDFYKKAGIWAGVDASQPVATVWADILNKLGKD